SEQUENCE LISTING (35 sequences)

SEQ ID NO: 1 - DNA, 3750 bp, Arabidopsis thaliana
  CAAT_signal (373)..(376)
  TATA_signal (424)..(428)
  intron (593)..(680)
  intron (919)..(1038)
  intron (1656)..(1761)
  intron (2537)..(2990)
aatatgtacatgcaataaaacatagtaatatatttctttccactatatatatatattgaattcaatgacttaaaacctttcaaaaaaatatttttgcttatataatcaagtgagttattggtaaagtgtatctttattttgaaaaaaaaactcattattttgaaaataaattatggttctctttacaaagaaatgatcaaagtttggtggacatatatatgtcaatcataagagagtcacaaactgagaatggagtttaaactaaagagctacaatattatccacaatttaaaacattttattaaaatcacgataacttcaaaaagagaaaatcaaaaattaactttgttaaaaaggtgggtatgaaaaatacaattttcttatttcctaacaaaaacaaaaatagaaacaaaggaaatgtgatataagaagattaaaagagacgttatgtctcacctatatttgctctctcctcttccttgtccaattctactgtcccaatccatcagttttatatggcaaactctcccgctgctcctgcacccaccaccacaaccggtggtgactcccggcgacgcctctccgcgtccatgtaagtgtatagtataatactctctaagtaatgattaaaaaaatctgaacaaaatcgtctaattgtggctttgtgtgtgtttaagcagagaagcaatatgcaagaggagattccggagaaatagcaaaggaggtggcagatcggatatggtgaaaccgtttaatatcataaatttttcgacacaagacaaaaacagtagttgttgttgtttcaccaagtttcagatcgtgaagcttctcttgtttatccttctctctgccactctcttcaccattatctattctcctgaagcttatcatcattctctttcccactcatcttctcggtaaatctatttcttttttccatcaccaacatttacattcttgacctcaaaaatgttcacatgcaaatttttacttttgcctctatctcttataatactatcttaaaattatgaaattagatggatatggagaagacaagatccacgttacttctcggatctggatataaactgggacgatgtgactaaaacccttgagaacatcgaagaaggccgtacgatcggtgtcttgaattttgattcgaacgagatccaacgatggagagaagtatccaagagcaaggacaatggggatgaagaaaaagttgttgtattgaatctagattacgcagacaagaatgtgacttgggacgcactatatccagagtggatcgatgaggagcaagaaacagaggtccctgtttgtcctaatatcccgaacattaaggtacctacaagaagactcgatctgatcgtcgtgaaacttccttgtcggaaagaagggaattggtcgagagacgtcgggagattgcatctacagctagcggctgcaactgtggcggcttcggccaaagggtttttcaggggacatgtgttttttgtatctagatgctttccgattccgaatcttttccggtgtaaagatcttgtgtctcggagaggcgatgtttggttgtacaaacctaatcttgataccttgagagacaagcttcagctgcctgtagggtcttgtgagctatctcttcctcttggcatccaaggtagaataaaaatgactcccgaaattacttgtttagatttgaaaacaaatttgaaaaatcgtcgctaagttaactagtgtctgttttcttccatgaattttacagataggccaagcttaggaaaccctaaaagagaagcttacgcaacaattcttcactcagctcacgtttacgtctgcggtgcaatcgccgcggctcagagcataagacagtctggttcgacgagagaccttgttatccttgttgatgacaacatcagcggttaccaccggagtggactagaagccgcgggttggcaaatccggacgatacagaggattcgaaaccctaaggcagagaaagatgcttacaacgaatggaactacagcaagttccggctatggcagctgactgattacgacaaaatcattttcatcgacgcggatctcttaatcttgagaaacatcgatttcttgttctcgatgcctgagatctcagctacaggaaacaatggaactctgtttaattcaggagttatggtgatcgagccttgcaactgtacgtttcagcttctgatggaacatataaacgagattgagtcttataacggtggagatcaaggttacttaaacgaggtattcacatggtggcaccggattccaaaacatatgaatttcttgaagcatttttggattggcgatgaagatgacgcgaaacgcaagaaaacagagctttttggagcagagcctcctgttctttatgttcttcattaccttgggatgaagccgtggttatgttaccgtgactacgactgtaacttcaactccgacatattcgttgagtttgctaccgatatcgctcatcgaaaatggtggatggtccacgacgccatgccacaggtgattcactctctcctaaaaaccttaatagaactcaaaaatcacataatattttcaatctcatattgtgatcaatattcaaaatattattaggcgtttagtcatgcgttgagagactaactgcatagcattatttctttctcaaaaatttccaaaacttgaaaaaataaataaactaaaaattacttactacccaagtttagaataaccatatgaaatttgaatatacgaaaatcttggtgggttagtaaatgcagaattagccccctacgcagtaggcatcaagttttaatgtctatgttttatacaccttataaaaaaatcatttcaaattttctttctttatgattagtttaaaaaaacattggttggcagaaatataaaaatagttagacgttttcccaaattattctaaaattgtgacggttagtaattaccatatatgatattttgcaggaacttcaccaattctgttacttgcgatccaagcaaaaggcacagctggaatatgatcgccggcaagcagaggccgcaaattatgccgacggtcattggaaaataagagtaaaggacccgagattcaaaatttgcatcgacaaattatgtaattggaaaagtatgctgcggcattggggcgaatcaaattggactgactacgagtcttttgttcccaccccaccagccattaccgtagaccggagatcatcacttcccggccataacttgtgacgcaataattatacatacttattaatggatttcatgagttttttggtttgaattgttgctgcgagattaggtgaatatcagttgtgtaactatatctttttcctatagtttgttcaaattgaataaaacatttttttgcagtttaaccacaaaataaaacatatgtcgtatttatatgccatttttgtatacaaacacaaactcaaaaatgttagtaacattcaaatagtttatacagaaacgatagattatagacttacatatagccaaacaacacaaattaattgatgtaactaaacatatgtagtataattaaactttcgaaaaatccaaatttttagtcgaatcgcagtgtagtatgtatacattacgtatagtatataaatctatgtgtgtgtatatcagtgtatgtatttgtgtatgtatgtacatgtgaaaagaatctctactaaagatttccataatattcaaccaaaaaccaaagtt

SEQ ID NO: 2 - DNA, 1980 bp, Arabidopsis thaliana
  CDS (1)..(1980)
  transit_peptide (1)..(195)
atggcaaactctcccgctgctcctgcacccaccaccacaaccggtggtgactcccggcgacgcctctccgcgtccatagaagcaatatgcaagaggagattccggagaaatagcaaaggaggtggcagatcggatatggtgaaaccgtttaatatcataaatttttcgacacaagacaaaaacagtagttgttgttgtttcaccaagtttcagatcgtgaagcttctcttgtttatccttctctctgccactctcttcaccattatctattctcctgaagcttatcatcattctctttcccactcatcttctcggtggatatggagaagacaagatccacgttacttctcggatctggatataaactgggacgatgtgactaaaacccttgagaacatcgaagaaggccgtacgatcggtgtcttgaattttgattcgaacgagatccaacgatggagagaagtatccaagagcaaggacaatggggatgaagaaaaagttgttgtattgaatctagattacgcagacaagaatgtgacttgggacgcactatatccagagtggatcgatgaggagcaagaaacagaggtccctgtttgtcctaatatcccgaacattaaggtacctacaagaagactcgatctgatcgtcgtgaaacttccttgtcggaaagaagggaattggtcgagagacgtcgggagattgcatctacagctagcggctgcaactgtggcggcttcggccaaagggtttttcaggggacatgtgttttttgtatctagatgctttccgattccgaatcttttccggtgtaaagatcttgtgtctcggagaggcgatgtttggttgtacaaacctaatcttgataccttgagagacaagcttcagctgcctgtagggtcttgtgagctatctcttcctcttggcatccaagataggccaagcttaggaaaccctaaaagagaagcttacgcaacaattcttcactcagctcacgtttacgtctgcggtgcaatcgccgcggctcagagcataagacagtctggttcgacgagagaccttgttatccttgttgatgacaacatcagcggttaccaccggagtggactagaagccgcgggttggcaaatccggacgatacagaggattcgaaaccctaaggcagagaaagatgcttacaacgaatggaactacagcaagttccggctatggcagctgactgattacgacaaaatcattttcatcgacgcggatctcttaatcttgagaaacatcgatttcttgttctcgatgcctgagatctcagctacaggaaacaatggaactctgtttaattcaggagttatggtgatcgagccttgcaactgtacgtttcagcttctgatggaacatataaacgagattgagtcttataacggtggagatcaaggttacttaaacgaggtattcacatggtggcaccggattccaaaacatatgaatttcttgaagcatttttggattggcgatgaagatgacgcgaaacgcaagaaaacagagctttttggagcagagcctcctgttctttatgttcttcattaccttgggatgaagccgtggttatgttaccgtgactacgactgtaacttcaactccgacatattcgttgagtttgctaccgatatcgctcatcgaaaatggtggatggtccacgacgccatgccacaggaacttcaccaattctgttacttgcgatccaagcaaaaggcacagctggaatatgatcgccggcaagcagaggccgcaaattatgccgacggtcattggaaaataagagtaaaggacccgagattcaaaatttgcatcgacaaattatgtaattggaaaagtatgctgcggcattggggcgaatcaaattggactgactacgagtcttttgttcccaccccaccagccattaccgtagaccggagatcatcacttcccggccataacttgtga

SEQ ID NO: 3 - protein, 659 aa, Arabidopsis thaliana
MANSPAAPAPTTTTGGDSRRRLSASIEAICKRRFRRNSKGGGRSDMVKPFNIINFSTQDKNSSCCCFTKFQIVKLLLFILLSATLFTIIYSPEAYHHSLSHSSSRWIWRRQDPRYFSDLDINWDDVTKTLENIEEGRTIGVLNFDSNEIQRWREVSKSKDNGDEEKVVVLNLDYADKNVTWDALYPEWIDEEQETEVPVCPNIPNIKVPTRRLDLIVVKLPCRKEGNWSRDVGRLHLQLAAATVAASAKGFFRGHVFFVSRCFPIPNLFRCKDLVSRRGDVWLYKPNLDTLRDKLQLPVGSCELSLPLGIQDRPSLGNPKREAYATILHSAHVYVCGAIAAAQSIRQSGSTRDLVILVDDNISGYHRSGLEAAGWQIRTIQRIRNPKAEKDAYNEWNYSKFRLWQLTDYDKIIFIDADLLILRNIDFLFSMPEISATGNNGTLFNSGVMVIEPCNCTFQLLMEHINEIESYNGGDQGYLNEVFTWWHRIPKHMNFLKHFWIGDEDDAKRKKTELFGAEPPVLYVLHYLGMKPWLCYRDYDCNFNSDIFVEFATDIAHRKWWMVHDAMPQELHQFCYLRSKQKAQLEYDRRQAEAANYADGHWKIRVKDPRFKICIDKLCNWKSMLRHWGESNWTDYESFVPTPPAITVDRRSSLPGHNL

SEQ ID NO: 4 - DNA, 560 bp, Zea mays
aaaattagcagcagccacagcaagaggcaatagaggaattcatgtgctgtttctgactgattgcttcccaattccaaacctcttctcttgcaaggacctagtgaaacgtgaaggcaatgcttggatgtacaaacctgacgtgaaggctctaaaggagaagctcaggctgcctgttggttcctgtgagcttgctgttccactcaacgcaaaagcacgactctacacggtagacagacgcagagaagcatatgctacaatactgcattcagcaagtgaatatgtttgcggtgcgataacagcagctcaaagcattcgtcaagcaggatcaacaagagaccttgttattcttgttgatgacaccataagtgactaccaccgcaaggggctggaatctgctgggtggaaggttagaataatacagaggatccggaatcccaaagcggaacgtgatgcctacaacgaatggaactacagcaaattccggctgtggcagcttacagattacgacaaggttattttcattgatgctgatctgctcatcctgaggaacattgatttct

SEQ ID NO: 5 - DNA, 1034 bp, Zea mays
gacgcgtacaacgagtggaactacagcaagttcaggctgtggcagctgaccgactacgacaaggtcatcttcatagacgccgacctcctcatcctgaggaacgtcgacttcctgttcgccatgccggagatcgccgcgacggscaacaacgccacgctcttcaactccggcgtcatggtcgtcgagccctccaactgcacgttccgcctgctcatggaccacatcgacgagatcacctcgtacaacggcggggaccaggggtacctcaacgagatattcacgtggtggcaccgcgtccccaggcacatgaacttcctcaagcacttctgggagggcgacagcgaggccatgaaggcgaagaagacacagctgttcggcgcggacccgccggtcctctacgtcctccactaccttggcctcaagccgtggctgtgcttcagagactacgactgcaactggaacaacgccgggatgcgcgagttcgccagcgacgtcgcgcatgcccggtggtggaaggtgcacgacaggatgccccggaagctccagtcctactgcctgctgaggtcgcggcagaaggccaggctggagtgggaccggaggcaggccgagaaggccaactctcaagatggccactggcgcctcaacgtcacggacaccaggctcaagacgtgctttgagaagttctgcttctgggagagcatgctctggcattggggcgagaacagtaacaggaccaagagcgtccccatggcagccacgacggcaaggtcgtgatctgtagatatacgaacaccccatccccatatggcaaccatacatgcatagcaatagcttgtataggtagctatgctttagttcttcgctatatatacagaatacaccactcgatccctgttgttgtcaaggctgcagctctatgtcgctgccggcctgccaccatggctaacgattcttttgggttggctgctgtaataagtttcaggtacatgtaaatttccctgctgaaattacgtgaccgcgttgagaaatgaatt

SEQ ID NO: 6 - DNA, 3606 bp, Arabidopsis thaliana
  CDS (1)..(3606)
atgtgtgtcaacttctctagtctgaaacttgttttgtttcttatgatgctggttgctatgttcacactctactgttctccaccgttgcaaattcctgaagatccatcaagttttgcaaacaaatggatactagaacctgctgtaaccacagatcctcgctatatagctacatctgagatcaactggaacagtatgtcacttgttgttgagcattacttatctggcagaagcgagtatcaaggaattggctttctaaatctcaacgataacgagattaatcgatggcaggtggtcataaaatctcactgtcagcatatagctttgcatctagaccatgctgcaagtaacataacttggaaatctttatacccggaatggattgacgaggaagaaaaattcaaagtccccacttgtccttctcttccttggattcaagttcctgacaagtctcgaatcgatcttatcattgccaagctcccatgtaacaagtcaggaaaatggtcaagagatgtggctagattgcacttacaacttgcagcagctcgagtggcggcatcttctgaagggcttcatgatgttcatgtgattttggtatcagattgctttccaataccgaatctttttacgggtcaagaacttgttgcccgtcaaggaaacatatggctgtataagcctaaacttcaccagttaagacaaaagttacaacttcctgttggttcctgtgaactttctgttcctcttcaagctaaagataatttctactcggcaaatgccaagaaagaagcgtacgcgacgatcttgcactcagatgatgcttttgtctgtggagccattgcagtagcacagagcattcgaatgtcaggctctactcgcaatttggtaatactagtcgatgattcgatcagtgaataccatagaagtggcttggaatcagctggatggaagattcacacatttcaaagaatcagaaacccgaaagctgaagcaaatgcatataaccaatggaactacagcaaattccgtctttgggaattgacagaatacaacaagatcatcttcattgatgcagacatgcttatcctcagaaacatggatttcctcttcgagtaccccgaaatctccacaactggaaacgacggtacgctcttcaactccggtctaatggtgattgaaccatcaaattcaacattccagttactaatggatcacatcaacgatatcaattcctacaatggaggagaccaaggttaccttaacgagatattcacatggtggcatcggattccaaaacacatgaatttcttgaagcatttctgggaaggagacacacctaagcacaggaaatctaagacgagactatttggagctgatcctccgatactctacgttcttcattacctaggttacaacaaaccatgggtatgcttcagagactacgattgcaattggaatgtcgttggataccatcaattcgcgagcgatgaagcacacaaaacttggtggagagtgcacgacgcgatgcctaagaaattgcagaggttttgtctactgagttcgaaacaaaaggcgcaacttgagtgggatcggagacaagctgagaaagcgaattacagagacggacattggaggattaagatcaaagataagagacttacgacttgttttgaagatttctgtttctgggagagtatgctttggcattggggcgattatgaaattctcgaaaccgaccctggtcttacggagacgatgataccttcctcaagtcccatggagtcaagacatcgactctcgttctcaaatgagaagacaagtaggaggagatttcaaagaattgagaagggtgtcaagttcaacactctgaaacttgtgttgatttgtataatgcttggagctttgttcacgatctaccgttttcgttatccaccgctacaaattcctgaaattccaactagttttggtcttactactgatcctcgctatgtagctacagctgagatcaactggaaccatatgtcaaatcttgttgagaagcacgtatttggtagaagcgagtatcaaggaattggtcttataaatcttaacgataacgagattgatcgattcaaggaggtaacgaaatctgactgtgatcatgtagctttgcatctagattatgctgcaaagaacataacatgggaatctttatacccggaatggattgatgaagttgaagaattcgaagtccctacttgtccttctctgcctttgattcaaattcctggcaagcctcggattgatcttgtaattgccaagcttccgtgtgataaatcaggaaaatggtctagagatgtggctcgcttgcatttacaacttgcagcagctcgagtggcggcttcttctaaaggacttcataatgttcatgtgattttggtatctgattgctttccaataccgaatctttttacgggtcaagaacttgttgcccgtcaaggaaacatatggctgtataagcctaatcttcaccagctaagacaaaagttacagcttcctgttggttcctgtgaactttctgttcctcttcaagctaaagataatttctactccgcaggtgcaaagaaagaagcttacgcgactatcttgcattctgcccaattttatgtctgtggagccattgcagctgcacagagcattcgaatgtcaggctctactcgtgatctggtcatacttgttgatgaaacgataagcgaataccataaaagtggcttggtagctgctggatggaagattcaaatgtttcaaagaatcaggaacccgaatgctgtaccaaatgcctacaacgaatggaactacagcaagtttcgtctttggcaactgactgaatacagtaagatcatcttcatcgatgcagacatgcttatcctgagaaacattgatttcctcttcgagttccctgagatatcagcaactggaaacaatgctacgctcttcaactctggtctaatggtggttgagccatctaattcaacattccagttactaatggataacattaatgaagttgtgtcttacaacggaggagaccaaggttaccttaacgagatattcacatggtggcatcggattccaaaacacatgaatttcttgaagcatttctgggaaggagacgaacctgagattaaaaaaatgaagacgagtctatttggagctgatcctccgatcctatacgttcttcattacctaggttataacaaaccctggttatgcttcagagactatgactgcaattggaatgtcgatattttccaggaatttgctagtgacgaggctcataaaacctggtggagagtgcacgacgcaatgcctgaaaacttgcataagttctgtctactaagatcgaaacagaaggcgcaacttgaatgggataggagacaagcagagaaagggaactacaaagatggacattggaagataaagatcaaagacaagagacttaagacttgtttcgaagatttctgcttttgggagagtatgctttggcattggggtgagacgaactctaccaacaattcttccaccaccaccacttcatcaccgccgcataaaaccgctctcccttccctgtga

SEQ ID NO: 7 - protein, 1201 aa, Arabidopsis thaliana
MCVNFSSLKLVLFLMMLVAMFTLYCSPPLQIPEDPSSFANKWILEPAVTTDPRYIATSEINWNSMSLVVEHYLSGRSEYQGIGFLNLNDNEINRWQVVIKSHCQHIALHLDHAASNITWKSLYPEWIDEEEKFKVPTCPSLPWIQVPDKSRIDLIIAKLPCNKSGKWSRDVARLHLQLAAARVAASSEGLHDVHVILVSDCFPIPNLFTGQELVARQGNIWLYKPKLHQLRQKLQLPVGSCELSVPLQAKDNFYSANAKKEAYATILHSDDAFVCGAIAVAQSIRMSGSTRNLVILVDDSISEYHRSGLESAGWKIHTFQRIRNPKAEANAYNQWNYSKFRLWELTEYNKIIFIDADMLILRNMDFLFEYPEISTTGNDGTLFNSGLMVIEPSNSTFQLLMDHINDINSYNGGDQGYLNEIFTWWHRIPKHMNFLKHFWEGDTPKHRKSKTRLFGADPPILYVLHYLGYNKPWVCFRDYDCNWNVVGYHQFASDEAHKTWWRVHDAMPKKLQRFCLLSSKQKAQLEWDRRQAEKANYRDGHWRIKIKDKRLTTCFEDFCFWESMLWHWGDYEILETDPGLTETMIPSSSPMESRHRLSFSNEKTSRRRFQRIEKGVKFNTLKLVLICIMLGALFTIYRFRYPPLQIPEIPTSFGLTTDPRYVATAEINWNHMSNLVEKHVFGRSEYQGIGLINLNDNEIDRFKEVTKSDCDHVALHLDYAAKNITWESLYPEWIDEVEEFEVPTCPSLPLIQIPGKPRIDLVIAKLPCDKSGKWSRDVARLHLQLAAARVAASSKGLHNVHVILVSDCFPIPNLFTGQELVARQGNIWLYKPNLHQLRQKLQLPVGSCELSVPLQAKDNFYSAGAKKEAYATILHSAQFYVCGAIAAAQSIRMSGSTRDLVILVDETISEYHKSGLVAAGWKIQMFQRIRNPNAVPNAYNEWNYSKFRLWQLTEYSKIIFIDADMLILRNIDFLFEFPEISATGNNATLFNSGLMVVEPSNSTFQLLMDNINEVVSYNGGDQGYLNEIFTWWHRIPKHMNFLKHFWEGDEPEIKKMKTSLFGADPPILYVLHYLGYNKPWLCFRDYDCNWNVDIFQEFASDEAHKTWWRVHDAMPENLHKFCLLRSKQKAQLEWDRRQAEKGNYKDGHWKIKIKDKRLKTCFEDFCFWESMLWHWGETNSTNNSSTTTTSSPPHKTALPSL

SEQ ID NO: 8 - DNA, 1653 bp, Arabidopsis thaliana
  CDS (1)..(1653)
atgggggccaaaagcaaaagttcgagtacgagattttttatgttttatcttatactaatatcattgtcgtttttgggtttgctcttaaactttaaacctctgtttctgctcaaccccatgatcgcttctccttcgatagttgagattcgttattctttgccggaaccggttaaacggactccgatatggctccgactcattagaaactatcttccggatgagaaaaagatccgagtgggtcttctcaacatcgcagagaacgagcgagagagctacgaggcaagcgggacgtcgatcttggagaatgtccacgtgtcgctcgatcctcttccgaacaatctgacatggacgagtttattcccggtttggatcgacgaggatcacacgtggcacattcctagttgtccagaagtccctctccctaagatggaaggttccgaagctgacgtggacgtcgtcgttgtcaaagtcccgtgcgatggtttctcggagaagagagggttaagagacgttttcaggctacaggtgaatctggcggcagcgaatcttgtggtggagagtggtcggaggaatgttgatcggactgtgtacgttgtcttcatcggatcttgtgggcctatgcatgagatctttaggtgtgatgagcgcgtgaagcgcgtgggggactattgggtctataggcctgatcttacgaggttgaagcagaagcttctcatgcctcctggttcatgtcagattgctccgctaggtcaaggagaagcatggatacaagacaagaacagaaatctcacatccgaaaaaactacattatcatcatttactgcccaacgtgtcgcttacgtgacgttactacactcatcggaggtatacgtatgcggagcaatagccttagcacaaagcataaggcaatctggatcaaccaaggacatgattctcctccacgatgactctataaccaacatctctctcattggcctaagccttgctggctggaaactacggcgagtggagagaattcgtagtcctttttccaagaagcgttcttacaatgagtggaactacagtaagttacgtgtgtggcaagtgacagattacgacaaactagtgtttatagacgcagacttcatcatcgtcaagaatattgattaccttttctcctatcctcaactttctgccgctggcaataacaaagtcttgttcaactcaggagtcatggttctggagccatcagcttgtttattcgaggatttgatgcttaaatcattcaagatcgggtcatacaacgggggagaccaaggatttctgaacgaatatttcgtgtggtggcataggcatgataaagcgcgcaatcttccagaaaatttagagggcatacactacttgggactaaaaccatggcgatgttacagagactacgattgtaactgggacttgaaaacgcgacgtgtgtatgcaagcgagtcggtgcatgcgagatggtggaaagtgtacgacaagatgcctaagaagctgaaaggttattgtggtttgaatcttaagatggagaagaacgttgagaagtggaggaaaatggctaagctcaatggttttcctgaaaatcattggaaaattagaataaaagatcctaggaagaagaaccgtctaagtcaatga

SEQ ID NO: 9 - protein, 550 aa, Arabidopsis thaliana
MGAKSKSSSTRFFMFYLILISLSFLGLLLNFKPLFLLNPMIASPSIVEIRYSLPEPVKRTPIWLRLIRNYLPDEKKIRVGLLNIAENERESYEASGTSILENVHVSLDPLPNNLTWTSLFPVWIDEDHTWHIPSCPEVPLPKMEGSEADVDVVVVKVPCDGFSEKRGLRDVFRLQVNLAAANLVVESGRRNVDRTVYVVFIGSCGPMHEIFRCDERVKRVGDYWVYRPDLTRLKQKLLMPPGSCQIAPLGQGEAWIQDKNRNLTSEKTTLSSFTAQRVAYVTLLHSSEVYVCGAIALAQSIRQSGSTKDMILLHDDSITNISLIGLSLAGWKLRRVERIRSPFSKKRSYNEWNYSKLRVWQVTDYDKLVFIDADFIIVKNIDYLFSYPQLSAAGNNKVLFNSGVMVLEPSACLFEDLMLKSFKIGSYNGGDQGFLNEYFVWWHRHDKARNLPENLEGIHYLGLKPWRCYRDYDCNWDLKTRRVYASESVHARWWKVYDKMPKKLKGYCGLNLKMEKNVEKWRKMAKLNGFPENHWKIRIKDPRKKNRLSQ

SEQ ID NO: 10 - DNA, 1674 bp, Arabidopsis thaliana
  CDS (1)..(1674)
atggggacaaaaacccataattctagagggaaaatcttcatgatctatctaatcctagtctcattgtcacttctaggtttgatcttaccttttaaacctcttttccggattacttctccatcttcaacgttacggattgatcttccatcgccgcaagtcaacaaaaacccgaaatggcttcgactcatccgtaactatctaccagagaaaagaatccaagtcggcttccttaacatagacgagaaagagcgtgagagctacgaggctcgtggaccgttggtacttaagaacatccacgtgccgcttgatcatatacccaagaatgtcacttggaagagtctttacccggagtggatcaacgaggaagcttctacctgtccggagatccctctccctcagccagaaggttctgatgctaacgtggacgttattgttgctagagttccatgtgatggttggtcggcgaataaagggcttagggacgtttttaggcttcaggttaatttggccgcagcgaatctagccgtccaaagtgggttgaggacggttaatcaggcggtctacgttgtattcatcggctcatgtgggcctatgcatgagattttcccgtgcgatgagcgcgtgatgcgcgtggaggattattgggtgtataagccttatctcccaaggttgaagcagaagcttctcatgcctgttggttcatgtcagattgctccttcatttgctcaatttggtcaagaagcatggagaccaaaacatgaagataatcttgcatcaaaggcagtcacagccttaccccgtcgcttacgggttgcctacgtgacagtactacactcgtcagaagcctatgtttgtggggcaatagctttagcgcaaagtataagacaatcaggatcgcataaggacatgattctcctccatgatcataccataaccaacaagtctcttattggtctcagcgctgcgggatggaatctccggctaatcgacaggatccgcagtcctttttcgcaaaaagactcttataatgagtggaactatagcaaattacgtgtgtggcaagtaactgactacgataaacttgtgttcatagacgcagatttcatcatcctcaagaaacttgatcatctcttctactatccacaactctcagcttcaggcaacgacaaagtgttattcaactccggaatcatggttctcgagccatcggcatgtatgtttaaagatttaatggagaaatcgttcaagattgagtcatacaacggaggagaccaaggattccttaatgagatatttgtatggtggcacaggttatcgaaacgagtgaacacaatgaagtacttcgacgaaaaaaatcatcgaagacacgatcttcctgagaatgtagaaggtctgcactacttggggttgaaaccatgggtatgttatagagactatgattgcaattgggacattagcgaacgacgcgtgtttgcaagcgattctgtgcacgaaaaatggtggaaagtgtatgacaaaatgtcagagcagttgaaaggttattgtggtttgaataagaatatggagaagaggattgagaagtggagaagaatcgctaagaacaatagtttgcctgataggcattgggagattgaagtgagagatcctaggaagacgaatcttcttgttcagtga

SEQ ID NO: 11 - protein, 557 aa, Arabidopsis thaliana
MGTKTHNSRGKIFMIYLILVSLSLLGLILPFKPLFRITSPSSTLRIDLPSPQVNKNPKWLRLIRNYLPEKRIQVGFLNIDEKERESYEARGPLVLKNIHVPLDHIPKNVTWKSLYPEWINEEASTCPEIPLPQPEGSDANVDVIVARVPCDGWSANKGLRDVFRLQVNLAAANLAVQSGLRTVNQAVYVVFIGSCGPMHEIFPCDERVMRVEDYWVYKPYLPRLKQKLLMPVGSCQIAPSFAQFGQEAWRPKHEDNLASKAVTALPRRLRVAYVTVLHSSEAYVCGAIALAQSIRQSGSHKDMILLHDHTITNKSLIGLSAAGWNLRLIDRIRSPFSQKDSYNEWNYSKLRVWQVTDYDKLVFIDADFIILKKLDHLFYYPQLSASGNDKVLFNSGIMVLEPSACMFKDLMEKSFKIESYNGGDQGFLNEIFVWWHRLSKRVNTMKYFDEKNHRRHDLPENVEGLHYLGLKPWVCYRDYDCNWDISERRVFASDSVHEKWWKVYDKMSEQLKGYCGLNKNMEKRIEKWRRIAKNNSLPDRHWEIEVRDPRKTNLLVQ

SEQ ID NO: 12 - DNA, 1002 bp, Arabidopsis thaliana
  CDS (1)..(1002)
atggccttactaaatgaattaatgagtttttttatccaaaaacaaaaagcaggtgtagacaaagtgtatgacctaacgaagatagaagcagagacaaaacgaccaaaacgtgaagcctacgtaactgttcttcactcttccgagtcttatgtctgtggtgccataactttggctcaaagcctccttcagacaaacaccaaacgcgatcttatccttctccacgatgactccatctccattaccaaacttcgagctctcgccgccgcaggatggaagcttcgtcggatcattcgaatcagaaacccacttgcggagaaggactcgtacaatgaatacaactacagcaagtttcgactctggcaattgacagattacgacaaagtgatcttcattgatgccgacatcatcgtcttacgtaaccttgatcttctcttccattttcctcagatgtcggccaccggaaatgatgtatggatatataattcaggcatcatggtcatcgagccttctaattgtacgtttactacaatcatgagccagcgaagcgagatcgtttcatacaacggtggagatcaagggtacctaaacgagatatttgtgtggtggcaccgattgcctcgacgagtaaactttctgaagaacttctggtcgaacacaaccaaagaaagaaacatcaagaacaacctcttcgccgcggagccgcctcaggtctacgcggtccactacttaggttggaaaccatggctttgctatagggactacgattgcaactacgacgtggacgagcagttggtgtacgctagtgatgcggctcacgttaggtggtggaaagtgcacgactccatggacgatgcattgcaaaagttttgcaggctgacgaaaaagaggagaacggagatcaactgggagaggaggaaagcaaggcttagaggttccactgattatcattggaagatcaatgtcactgatccaagacgacgtcgttcttatttgattggttaa

SEQ ID NO: 13 - protein, 333 aa, Arabidopsis thaliana
MALLNELMSFFIQKQKAGVDKVYDLTKIEAETKRPKREAYVTVLHSSESYVCGAITLAQSLLQTNTKRDLILLHDDSISITKLRALAAAGWKLRRIIRIRNPLAEKDSYNEYNYSKFRLWQLTDYDKVIFIDADIIVLRNLDLLFHFPQMSATGNDVWIYNSGIMVIEPSNCTFTTIMSQRSEIVSYNGGDQGYLNEIFVWWHRLPRRVNFLKNFWSNTTKERNIKNNLFAAEPPQVYAVHYLGWKPWLCYRDYDCNYDVDEQLVYASDAAHVRWWKVHDSMDDALQKFCRLTKKRRTEINWERRKARLRGSTDYHWKINVTDPRRRRSYLIG

SEQ ID NO: 14 - DNA, 834 bp, Arabidopsis thaliana
  CDS (1)..(834)
atggctccttccaaatctgcactgatacgctttaatctagtcttgttggcagcggagcttcctttgttggatgctcttttcgtgattgcactcccaagactaatagatatctttatactgctatgtgatcaggtggtgagaggagtgaagatgcaagaactcgttgaagagaacgaaataaacaagaaagatttgctaaccgctagtaaccagacaaagctggaggcgccaagcttcatggaagagattttaacaagagggttaggaaaaacaaagatagggatggtgaacatggaagaatgtgatcttactaattggaaacgttatggcgaaacggttcacatacattttgagcgtgtctcgaagctcttcaaatggcaagacttgttccccgagtggatagatgaagaggaagaaaccgaggttcccacatgtcctgagatacctatgcccgatttcgaaagcttagagaagttggatttggtagtagtgaagttgccttgtaattaccctgaagaagggtggagaagagaggttttgaggttgcaagtgaacctagttgcggctaacttggcagccaagaaagggaagacggattggagatggaaaagcaaagtgttgttttggagcaaatgtcaaccgatgattgagattttccggtgtgatgatttggagaagagagaggcagattggtggctgtatcgccctgaggtggttaggttacaacagagactcagtttgccagtcggatcttgcaatcttgctcttcctttgtgggcaccacaaggtaaaattactttcatgcaaattaatcttcttgctaaatatttttag

SEQ ID NO: 15 - protein, 277 aa, Arabidopsis thaliana
MAPSKSALIRFNLVLLAAELPLLDALFVIALPRLIDIFILLCDQVVRGVKMQELVEENEINKKDLLTASNQTKLEAPSFMEEILTRGLGKTKIGMVNMEECDLTNWKRYGETVHIHFERVSKLFKWQDLFPEWIDEEEETEVPTCPEIPMPDFESLEKLDLVVVKLPCNYPEEGWRREVLRLQVNLVAANLAAKKGKTDWRWKSKVLFWSKCQPMIEIFRCDDLEKREADWWLYRPEVVRLQQRLSLPVGSCNLALPLWAPQGKITFMQINLLAKYF

SEQ ID NO: 16 - DNA, 383 bp, Hordeum vulgare
  CDS (46)..(381)
ttgaatctgcgggttggaaggtcagaataattgagaggatcggaacccgaagccgagcgtgatgcttacaatgagtggaactacagcaagttccggttgtggcagctcacggactatgacaagatcatattcatagatgctgatctgctcatcttgaggaacattgatttcctgtttacaatgccagaaatcagtgcaaccggcaacaatgcaacactcttcaactctggtgtcatggtcatcgaaccctcaaactgcacattccagctgttaatggagcacatcaatgagataacatcttacaatggtggtgatcagggctacttgaatgagatattcacatggtggcatcggattcccaagcacatgaacttcctgaagca

SEQ ID NO: 17 - protein, 112 aa, Hordeum vulgare
PEAERDAYNEWNYSKFRLWQLTDYDKIIFIDADLLILRNIDFLFTMPEISATGNNATLFNSGVMVIEPSNCTFQLLMEHINEITSYNGGDQGYLNEIFTWWHRIPKHMNFLK

SEQ ID NO: 18 - DNA, 245 bp, Hordeum vulgare
  CDS (52)..(243)
cgagcttgaatctgcgggttggcaagtcagaataattgagaggatccggaacccgaagccgagcgtgatgcttacaatgagtggaactacagcaagttccggttgtggcagctcacggactatgacaagatcatattcatagatgctgatctgctcatcttgaggaacattgatttcctgtttacaatgccagaaatcagtgcaaacggcaacaatgcaacactcttcaactctggtgtcatggt

SEQ ID NO: 19 - protein, 64 aa, Hordeum vulgare
PEAERDAYNEWNYSKFRLWQLTDYDKIIFIDADLLILRNIDFLFTMPEISANGNNATLFNSGVM

SEQ ID NO: 20 - DNA, 1284 bp, Triticum aestivum
  CDS (1)..(1284)
  Variant 272: Xaa = Ochre
acgcgtccgctcgccttcttcttcctcgttctacatggccctcctgctccaccccaagtactcccacatcctcgaccgcggcgcctcctctctggtccgctgcaccttccgcgacgcctgcccgtccacgtcccacctctcacggaaggtaagccgggaggaagatcagtggcggcggcgaacaaggtggtggcgacggagcggatcgtgaacgcggggcgcgcgccgaccatgttcaacgagctgcgcggccggctgcggatgggcctggtgaacatcggccgcgacgagctgctggcgctgggcgtggagggagacgccgtgggcgtggacttcgaccgcgtgtcggacgtgttccggtggtcagacctgttcccggagtggatcgacgaggaggaggaggacggcgtcccctcctgcccggagatccccatgccggacttctcccggtacgacgacgacggcgtggacgtggtggtggcggcgctgccgtgcaaccggacggcggtccgggggtggaaccgcgacgtgttcaggctgcaggtgcacctggtggcggcgcacatggcggcgcggaagtgggcggcgcgacggcgccggccgggtgcgcgtggtgctgcggagcgagtgcgagccgatgatggacctgttccggtgcgacgagtccgtggggcgggagggggactggtggatgtacagcgtcgacgcgccgcgcatggaggagaagctccggctgcccatcggctcctgcaacctcgccgctgccgctctgggggccaacaggcatccacgaggtgttcaacgcgtcagacctaacagcggtggacgccggcagccagcggcgcgaggcgtacgcgactggtgctgcactcgtccgaccgatacctgtgcggcgccatcgtgctggcgcagagcatccggcggtcgggctccacccgcgacatggtcctcctccacgaccacaccgtctccaagccggccctccgcgcgctggtcgccgccggctggatcccgcgcaggatccggcgcatccgcaacccgcgcgcggagcggggctcctacaacgagtacaactacagcaagttccggctgtggcagctgacggagtacttccgcgtcgtcttcatcgacgccgacatcctcgtcctccgctccctcgacgcgctcttccgcttcccgcagatctccgccgggggcaacgacggctccctcttcaactcggggaacatggtgctcgagccgtcggcgtgcaccttcgaggcgctcgtccgggggcggcgcaca

SEQ ID NO: 21 - protein, 271 aa, Triticum aestivum
TRPLAFFFLVLHGPPAPPQVLPHPRPRRLLSGPLHLPRRLPVHVPPLTEGKPGGRSVAAANKVVATERIVNAGRAPTMFNELRGRLRMGLVNIGRDELLALGVEGDAVGVDFDRVSDVFRWSDLFPEWIDEEEEDGVPSCPEIPMPDFSRYDDDGVDVVVAALPCNRTAVRGWNRDVFRLQVHLVAAHMAARKWAARRRRPGARGAAERVRADDGPVPVRRVRGAGGGLVDVQRRRAAHGGEAPAAHRLLQPRRCRSGGQQASTRCSTRQT

SEQ ID NO: 22 - protein, 156 aa, Triticum aestivum
QRWTPAASGARRTRLVLHSSDRYLCGAIVLAQSIRRSGSTRDMVLLHDHTVSKPALRALVAAGWIPRRIRRIRNPRAERGSYNEYNYSKFRLWQLTEYFRVVFIDADILVLRSLDALFRFPQISAGGNDGSLFNSGNMVLEPSACTFEALVRGRRT

SEQ ID NO: 23 - DNA, 2028 bp, Arabidopsis thaliana
  CDS (1)..(1854)
atgataccttcctcaagtcccatggagtcaagacatcgactctcgttctcaaatgagaagacaagtaggaggagatttcaaagaattgagaagggtgtcaagttcaacactctgaaacttgtgttgatttgtataatgcttggagctttgttcacgatctaccgttttcgttatccaccgctacaaattcctgaaattccaactagttttggtcttactactgatcctcgctatgtagctacagctgagatcaactggaaccatatgtcaaatcttgttgagaagcacgtatttggtagaagcgagtatcaaggaattggtcttataaatcttaacgataacgagattgatcgattcaaggaggtaacgaaatctgactgtgatcatgtagctttgcatctagattatgctgcaaagaacataacatgggaatctttatacccggaatggattgatgaagttgaagaattcgaagtccctacttgtccttctctgcctttgattcaaattcctggcaagcctcggattgatcttgtaattgccaagcttccgtgtgataaatcaggaaaatggtctagagatgtggctcgcttgcatttacaacttgcagcagctcgagtggcggcttcttctaaaggacttcataatgttcatgtgattttggtatctgattgctttccaataccgaatctttttacgggtcaagaacttgttgcccgtcaaggaaacatatggctgtataagcctaatcttcaccagctaagacaaaagttacagcttcctgttggttcctgtgaactttctgttcctcttcaagctaaagataatttctactccgcaggtgcaaagaaagaagcttacgcgactatcttgcattctgcccaattttatgtctgtggagccattgcagctgcacagagcattcgaatgtcaggctctactcgtgatctggtcatacttgttgatgaaacgataagcgaataccataaaagtggcttggtagctgctggatggaagattcaaatgtttcaaagaatcaggaacccgaatgctgtaccaaatgcctacaacgaatggaactacagcaagtttcgtctttggcaactgactgaatacagtaagatcatcttcatcgatgcagacatgcttatcctgagaaacattgatttcctcttcgagttccctgagatatcagcaactggaaacaatgctacgctcttcaactctggtctaatggtggttgagccatctaattcaacattccagttactaatggataacattaatgaagttgtgtcttacaacggaggagaccaaggttaccttaacgagatattcacatggtggcatcggattccaaaacacatgaatttcttgaagcatttctgggaaggagacgaacctgagattaaaaaaatgaagacgagtctatttggagctgatcctccgatcctatacgttcttcattacctaggttataacaaaccctggttatgcttcagagactatgactgcaattggaatgtcgatattttccaggaatttgctagtgacgaggctcataaaacctggtggagagtgcacgacgcaatgcctgaaaacttgcataagttctgtctactaagatcgaaacagaaggcgcaacttgaatgggataggagacaagcagagaaagggaactacaaagatggacattggaagataaagatcaaagacaagagacttaagacttgtttcgaagatttctgcttttgggagagtatgctttggcattggggtgagacgaactctaccaacaattcttccaccaccaccacttcatcaccgccgcataaaaccgctctcccttccctgtgaattcttttggctttctggtttggtacaaattactctgcctttcgccaaccaaatgtgggttggatatgttcttttgtttttttattatcagcttgaaacctgtatacgaatcccagaaacaatgtaatcatgaggggataaaggaatgaaagacaaataaagaatttacag

SEQ ID NO: 24 - protein, 618 aa, Arabidopsis thaliana
MIPSSSPMESRHRLSFSNEKTSRRRFQRIEKGVKFNTLKLVLICIMLGALFTIYRFRYPPLQIPEIPTSFGLTTDPRYVATAEINWNHMSNLVEKHVFGRSEYQGIGLINLNDNEIDRFKEVTKSDCDHVALHLDYAAKNITWESLYPEWIDEVEEFEVPTCPSLPLIQIPGKPRIDLVIAKLPCDKSGKWSRDVARLHLQLAAARVAASSKGLHNVHVILVSDCFPIPNLFTGQELVARQGNIWLYKPNLHQLRQKLQLPVGSCELSVPLQAKDNFYSAGAKKEAYATILHSAQFYVCGAIAAAQSIRMSGSTRDLVILVDETISEYHKSGLVAAGWKIQMFQRIRNPNAVPNAYNEWNYSKFRLWQLTEYSKIIFIDADMLILRNIDFLFEFPEISATGNNATLFNSGLMVVEPSNSTFQLLMDNINEVVSYNGGDQGYLNEIFTWWHRIPKHMNFLKHFWEGDEPEIKKMKTSLFGADPPILYVLHYLGYNKPWLCFRDYDCNWNVDIFQEFASDEAHKTWWRVHDAMPENLHKFCLLRSKQKAQLEWDRRQAEKGNYKDGHWKIKIKDKRLKTCFEDFCFWESMLWHWGETNSTNNSSTTTTSSPPHKTALPSL

SEQ ID NO: 25 - DNA, 1845 bp, Oryza sativa
  CDS (1)..(1845)
atgggggtgacgggcggcgccggggaggccgtcaagccgtcgtcgtcgtcgtcgttgtcgccggtggcggggctgagggcggcggccatcgtgaagctgaacgcggcgttcctcgccttcttcttcctcgcgtacatggcgctcctcctccaccccaagtactcctacctcctcgaccgcggcgccgcctcctccctcgtccgctgcaccgccttccgcgacgcctgcaccccggcgacgacgaccaccgcccagctctctcggaagctgggaggcgtggcggcgaacaaggcggtggcggcggcggcggagaggatcgtgaacgccgggagggcgccggcgatgttcgacgagctccgtgggcggctgcggatgggcctggtgaacatcggccgcgacgagctgctggcgctcggcgtggagggcgacgccgtcggcgtcgacttcgagcgcgtctccgacatgttccggtggtcggacctcttcccggagtggatcgacgaggaggaggacgacgagggcccgtcctgcccggagctccccatgccggacttctcccggtacggcgacgtcgacgtggtggtggcgtcgctgccgtgcaaccgttcggacgccgcgtggaaccgcgacgtgttcaggctgcaggtgcacctcgtgacggcgcacatggcggcgcgcaaggggctgcggcacgacgccggcggcggcggcggcggcgggcgggtgcgcgtggtggtgcgcagcgagtgcgagcccatgatggacttgttccggtgcgacgaggcggtggggagggacggcgagtggtggatgtacatggtcgacgtcgagcggctggaggagaagctccggcttcctgtcggctcatgcaacctcgccctacctctgtggggacccggaggtatccaggaagtgttcaacgtgtcggagctgacggcggcggcggcaacggcggggcggccgcggcgggaggcgtacgcgacggtgctccactcgtcggacacgtacctgtgcggcgcgatcgtgctggcgcagagcatccggcgcgccgggtcgacgcgcgacctcgtcctcctccacgaccacaccgtgtcgaagccggcgctggcggcgctggtcgccgccggctggaccccgcgcaagatcaagcgcatccgcaacccgcgcgcggagcgcggcacctacaacgagtacaactacagcaagttccggctgtggcagctcaccgactacgaccgcgtggtgttcgtcgacgccgacatcctcgtcctccgcgacctcgacgccctcttcggcttcccgcagctgacggcggtgggcaacgacggctcgctcttcaactccggggtgatggtgatcgagccgtcgcagtgcacgttccagtcgctgatccggcagcggcggaccatccggtcctacaacggcggcgatcaggggttcctgaacgaggtgttcgtctggtggcaccggctgccgcggcgggtgaactacctcaagaacttctgggcgaacactacggcggagcgggcgctcaaggagcggctgttccgggcggatcccgcggaggtgtggtcgatccactacctggggctgaagccgtggacgtgctaccgcgactacgactgcaactggaacatcggcgaccagcgggtgtacgccagcgacgccgcgcacgcgcggtggtggcaggtgtacgacgacatgggggaggccatgcgctcgccgtgccgcctgtcggagcggaggaagatcgagatcgcctgggaccgacacctcgccgaggaggccggcttctccgaccaccactggaagatcaacatcaccgacccccgcaagtgggagtag

SEQ ID NO: 26 - protein, 614 aa, Oryza sativa
MGVTGGAGEAVKPSSSSSLSPVAGLRAAAIVKLNAAFLAFFFLAYMALLLHPKYSYLLDRGAASSLVRCTAFRDACTPATTTTAQLSRKLGGVAANKAVAAAAERIVNAGRAPAMFDELRGRLRMGLVNIGRDELLALGVEGDAVGVDFERVSDMFRWSDLFPEWIDEEEDDEGPSCPELPMPDFSRYGDVDVVVASLPCNRSDAAWNRDVFRLQVHLVTAHMAARKGLRHDAGGGGGGGRVRVVVRSECEPMMDLFRCDEAVGRDGEWWMYMVDVERLEEKLRLPVGSCNLALPLWGPGGIQEVFNVSELTAAAATAGRPRREAYATVLHSSDTYLCGAIVLAQSIRRAGSTRDLVLLHDHTVSKPALAALVAAGWTPRKIKRIRNPRAERGTYNEYNYSKFRLWQLTDYDRVVFVDADILVLRDLDALFGFPQLTAVGNDGSLFNSGVMVIEPSQCTFQSLIRQRRTIRSYNGGDQGFLNEVFVWWHRLPRRVNYLKNFWANTTAERALKERLFRADPAEVWSIHYLGLKPWTCYRDYDCNWNIGDQRVYASDAAHARWWQVYDDMGEAMRSPCRLSERRKIEIAWDRHLAEEAGFSDHHWKINITDPRKWE

SEQ ID NO: 27 - DNA, 626 bp, Zea mays
  CDS (133)..(624)
ttcgagcggccgccccgggcaggtacaaacctgacgtgaaggctctaaaggagaagctcaggctgcctgttggttcctgtgagcttgctgttccactcaacgcaaaagcacgactcttacacggtagacagacgcagagaagcatatgctacaatacttcattcagcaagtgaatatgtttgcggtgcgataacagcagctcaaagcattcgtcaagcaggatcaacaagagaccttgttattcttgttgatgacaccataagtgaccaccaccgcaaggggctggaatctgctgggtggaaggtcagaataatagaaaggatccggaatcccaaagccgaacgtgatgcctacaacgaatggaactacagcaaattccggctgtggcagcttacagattacgacaaggttattttcattgatgctgatctgctcatcctgaggaacattgatttcttgtttgcaatgccagaaatcaccgcaactgggaacaatgccacactcttcaactctggggtgatggtcattgaaccttcaaactgcacgttccagttactgatggagcacatcaacgagataacatcttacaacggtggtgaccaagggtacctcggccgcgaccacgc

SEQ ID NO: 28 - protein, 164 aa, Zea mays
RREAYATILHSASEYVCGAITAAQSIRQAGSTRDLVILVDDTISDHHRKGLESAGWKVRIIERIRNPKAERDAYNEWNYSKFRLWQLTDYDKVIFIDADLLILRNIDFLFAMPEITATGNNATLFNSGVMVIEPSNCTFQLLMEHINEITSYNGGDQGYLGRDH

SEQ ID NO: 29 - DNA, 553 bp, Zea mays
  CDS (1)..(552)
tggaaggtcagaataatagaaaggatccggaatcccaaagccgaacgtgatgcctacaacgaatggaactacagcaaattccggctgtggcagcttacagattacgacaaggttattttcattgatgctgatctgctcatcctgaggaacattgatttcttgtttgcaatgccagaaatcaccgcaactgggaacaatgccacactcttcaactctggggtgatggtcattgaaccttcaaactgcacgttccagttactgatggagcacatcaacgagataacatcttacaacggtggtgaccaagggtacctgaacgagatattcacatggtggcaccggattccaaagcacatgaatttcttgaagcatttctgggagggtgatgaggacgaagtgaaggccaagaagactcggctgttcggcgccaacccaccgatcctctacgttctccactacttggggcggaagccatggctgtgcttccgggactacgattgcaactggaacgtcgagatcttgcgggagtttgcgagtgacgttgcgcatgccc

SEQ ID NO: 30 - protein, 184 aa, Zea mays
WKVRIIERIRNPKAERDAYNEWNYSKFRLWQLTDYDKVIFIDADLLILRNIDFLFAMPEITATGNNATLFNSGVMVIEPSNCTFQLLMEHINEITSYNGGDQGYLNEIFTWWHRIPKHMNFLKHFWEGDEDEVKAKKTRLFGANPPILYVLHYLGRKPWLCFRDYDCNWNVEILREFASDVAHA

SEQ ID NO: 31 - DNA, 552 bp, Zea mays
  CDS (1)..(552)
tccctgcgccggctcagccccaacgccgaccgcgtcgtcatcgcgtccctcgacgtcccgccgctctgggttcaggcactgaaaaatgacggggtaaaggtggtctctgtggagaatttgaaaaatccttacgagaaacaagaaaatttcaacagacgattcaaattgactttaaacaagctgtatgcatggagcttggtttcatatgagcgagttgttatgcttgactctgacaacattttcctccaaaatactgatgagttatttcagtgtggtcagttctgtgctgtcttcatcaatccctgtatcttccatacaggtctttttgtgcttcagccctcaatggatgtttttaagaacatgctacatgagctagcggttggacgtgaaaacccagatggggcagaccaaggcttccttgctagttatttcccggacttgcttgatcagccaatgttccatccaccagctaatggtacaaaactttggggtacttatcgcctccccctaggctaccagatggatgcatcttactattatctgaagcttcgc

SEQ ID NO: 32 - protein, 184 aa, Zea mays
SLRRLSPNADRVVIASLDVPPLWVQALKNDGVKVVSVENLKNPYEKQENFNRRFKLTLNKLYAWSLVSYERVVMLDSDNIFLQNTDELFQCGQFCAVFINPCIFHTGLFVLQPSMDVFKNMLHELAVGRENPDGADQGFLASYFPDLLDQPMFHPPANGTKLWGTYRLPLGYQMDASYYYLKLR

SEQ ID NO: 33 - DNA, 560 bp, Zea mays
  CDS (1)..(558)
aaacctgacgtgaaggcgttgaaggagaagctcaggctgcctgttggttcctgtgagcttgctgttccactcaacgcaaaagcacgactctacacagtagacagacgcagagaagcatatgcgacaatactgcattcagcaagtgaatatgtttgcggcgcgatcacggcagctcaaagcattcgtcaagcaggatcaacaagagacctcgttattctcgtcgacgacaccataagtgaccaccaccgcaaggggctgcaatctgcggggtggaaggtcaggataatacagaggatccggaaccccaaagccgagcgcgacgcctacaacgagtggaactacagcaaattccggctgtggcagctcacggattacgacaaggtcatcttcatcgacgcggatctcctcatcctgaggaacatcgatttcctgttcgcgctgccggagatcacggcgacggggaacaacgcgacgctcttcaactcgggagtgatggtcatcgagccttcgaactgcacgttccggctactgatggagcacatcgacgagataacgtcgta

SEQ ID NO: 34 - protein, 186 aa, Zea mays
KPDVKALKEKLRLPVGSCELAVPLNAKARLYTVDRRREAYATILHSASEYVCGAITAAQSIRQAGSTRDLVILVDDTISDHHRKGLQSAGWKVRIIQRIRNPKAERDAYNEWNYSKFRLWQLTDYDKVIFIDADLLILRNIDFLFALPEITATGNNATLFNSGVMVIEPSNCTFRLLMEHIDEITS

SEQ ID NO: 35 - protein, 566 aa, Arabidopsis thaliana
MGAKSKSSSTRFFMFYLILISLSFLGLLLNFKPLFLLNPMIASPSIVEIRYSLPEPVKRTPIWLRLIRNYLPDEKKIRVGLLNIAENERESYEASGTSILENVHVSLDPLPNNLTWTSLFPVWIDEDHTWHIPSCPEVPLPKMEGSEADVDVVVVKVPCDGFSEKRGLRDVFRLQVNLAAANLVVESGRRNVDRTVYVVFIGSCGPMHEIFRCDERVKRVGDYWVYRPDLTRLKQKLLMPPGSCQIAPLGQGEAWIQDKNRNLTSEKTTLSSFTAQRVAYVTLLHSSEVYVCGAIALAQSIRQSGSTKDMILLHDDSITNISLIGLSLAGWKLRRVERIRSPFSKKRSYNEWNYSKLRVWQVTDYDKLVFIDADFIIVKNIDYLFSYPQLSAAGNNKVLFNSGVMVLEPSACLFEDLMLKSFKIGSYNGGDQGFLNEYFVWWHRLSKRLNTMKYFGDESRHDKARNLPENLEGIHYLGLKPWRCYRDYDCNWDLKTRRVYASESVHARWWKVYDKMPKKLKGYCGLNLKMEKNVEKWRKMAKLNGFPENHWKIRIKDPRKKNRLSE

(19) United States
(12) Patent Application Publication
(10) Pub. No.: US 2003/0200564 A1
(43) Pub. Date: Oct. 23, 2003

(54) Starch modification

(76) Inventors: Michael Meyrick Burrell, Cambridge (GB); Manash Chatterjee, Cambridge (GB)

Correspondence Address: Pennie & Edmonds LLP, 1155 Avenue of the Americas, New York, NY, 10036-2711, US

(21) Appl. No.: 10/214,529

(22) Filed: Aug. 8, 2002

Related U.S. Application Data
(60) Provisional application No. 60/346,907, filed on Jan. 8, 2002.

(30) Foreign Application Priority Data
Aug. 8, 2001 (GB) 0119342.4

(51) Int. Cl. A01H 1/00; C12Q 1/68; C07H 21/04; C12N 9/32; C12N 15/82; C12P 21/02; C12N 5/04

(52) U.S. Cl. 800/284; 435/6; 435/69.1; 435/320.1; 435/468; 435/419; 435/204; 536/23.2

(57) ABSTRACT

The present invention relates to a method of altering starch synthesis in a plant by modifying the starch priming activity of the plant. In particular, this is achieved by altering the expression or activity of a starch primer which is preferably encoded by the sequence of SEQ ID NO: 1 or a sequence substantially homologous thereto. Also provided are plants in which the starch priming activity has been altered and plant parts.

This application claims priority to U.S. Provisional Patent Application No. 60/346,907, filed on Jan. 8, 2002 and Great Britain Patent Application No. 0119342.4, filed on Aug. 8, 2001, both of which are incorporated by reference herein.

### 1 FIELD OF INVENTION

The present invention is based upon the identification of a protein, which initiates starch synthesis in a plant. In particular, the intention relates to plant glycogenin-like nucleic acid molecules, plant glycogenin-like gene products, antibodies to plant glycogenin-like gene products, plant glycogenin-like regulatory regions, vectors and expression vectors with plant glycogenin-like genes, cells, plants and plant parts with plant glycogenin-like genes, modified starch from such plants and the use of the foregoing to improve agronomically valuable plants.

### 2 BACKGROUND

Starch, a branched polymer of glucose consisting of largely linear amylose and highly branched amylopectin, is the product of carbon fixation during photosynthesis in plants, and is the primary metabolic energy reserve stored in seeds and fruit. For example, up to 75% of the dry weight of grain in cereals is made up of starch. The importance of starch as a food source is reflected by the fact that two thirds of the worlds food consumption (in terms of calories) is provided by the starch in grain crops such as wheat, rice and maize.

Starch is the product of photosynthesis, and is analogous to the storage compound glycogen in eukaryotes. It is produced in the chloroplasts or amyloplasts of plant cells, these being the plastids of photosynthetic cells and non-photosynthetic cells, respectively. The biochemical pathway leading to the production of starch in leaves has been well characterised, and considerable progress has also been made in elucidating the pathway of starch biosynthesis in storage tissues.

The biosynthesis of starch molecules is dependent on a complex interaction of numerous enzymes, including several essential enzymes such as ADP-Glucose, a series of starch synthases which use ADP glucose as a substrate for forming chains of glucose linked by alpha-1-4 linkages, and a series of starch branching enzymes that link sections of polymers with alpha-1-6 linkages to generate branched structures (Smith et al., 1995, Plant Physiology, 107:673-677). Further modification of the starch by yet other enzymes, i.e. debranching enzymes or disproportionating enzymes, can be specific to certain species.

The fine structure of starch is a complex mixture of D-glucose polymers that consist essentially of linear chains (amylose) and branched chains (amylopectin) glucans. Typically, amylose makes up between 10 and 25% of plant starch, but varies significantly among species. Amylose is composed of linear D-glucose chains typically 250-670 glucose units in length (Tester, 1997, in: Starch Structure and Functionality, Frazier et al., eds., Royal Society of Chemistry, Cambridge, UK). The linear regions of amylopectin are composed of low molecular weight and high molecular weight chains, with the low ranging from 5 to 30 glucose units and the high molecular weight chains from 30 to 100 or more. The amylose/amylopectin ratio and the distribution of low and high molecular weight D-glucose chains can affect starch granule properties such as gelatinization temperature, retrogradation, and viscosity (Blanshard, 1987). The characteristics of the fine structure of starch mentioned above have been examined at length and are well known in the art of starch chemistry.

It is know that starch granule size and amylose percentage change during kernel development in maize and during tobacco leaf development (Boyer et al., 1976, Cereal Chem 53:327-337). In his classic study Boyer et al. concluded the amylose percentage of starch decreases with decreasing granule size in later stages of maize kernel development.

As mentioned above, glycogen serves as the glucose reserve in animals rather than starch. The biosynthesis of glycogen in eukaryotes involves chain elongation through the formation of linear alpha-1,4 glycosidic linkages catalysed by the enzyme, glycogen synthase. Evidence for a distinct initiation step involving a self-glucosylating protein, known as glycogenin or SGP, came from work directed at mammalian systems (Smythe et al., Eur. J. Biochem 200:625-631 (1990) and Whelan Bioessays 5:136-140 (1986)).

Cheng et al (Mol. and Cell Biol. 15(12): 6632-6640 (1995)) report the identification of two yeast genes whose products are implicated in the biosynthesis of glycogen. The two genes, Glg1 and Glg2 encode self-glucosylating proteins which in vitro act as primers for the elongation reaction catalysed by glycogen synthase. Disruption of both these genes results in the inability to synthesise glycogen, despite normal levels of glycogen synthase. Glycogenin homologues have been identified in Caenorhabditis elegans and humans (Mu et al., J. Biol. Chem. 272(44): 27589-27597(1997)).

It is now well established that glycogen synthesis is initiated on the primer protein, glycogenin or SGP, which remains covalently attached to the resulting macromolecule. The initiation step is thought to involve glycogenin growing a covalently attached oligosaccharide primer linked via a unique carbohydrate-protein bond via the hydroxyl group of the Tyr residue, Tyr 194. Once this oligosaccharide chain on glycogenin has been extended sufficiently glycogen synthase is able to catalyse elongation and together with the branching enzyme, form the mature glycogen molecule (Rodriguez and Whelan, Biochem Biophy Res Comm, 132:829-836; Roach and Skurat, 1997, in Progress in Nucleic Acid Research and Molecular Biology p289-316, Academic Press).

Previous workers have set out to determine whether a priming molecule, such as a self glucosylating protein, is responsible for the initiation of starch synthesis in plants. WO94/04693 (Zeneca Ltd.) describes the purification of a putative starch priming protein molecule from maize endosperm, known as amylogenin, and isolation of a partial cDNA. The maize amylogenin showed no sequence homology with glycogenin and exhibited a novel glucose-protein bond (Singh et al., FEBS Letters 376: 61-64 (1995)). However, based upon the sequence homology and the reported properties of the maize protein, it has subsequently been shown that the sequence of the maize nucleic acid molecule reported above is homologous to a reversibly glycosylated polypeptide (RGP 1) from pea (Dhugga et al., Proc. Natl. Acad. Sci. USA 94:7679-7684 (1997)). RGP1 is localised to the Golgi apparatus and is thought to be involved in cell wall synthesis. This has dispelled the initial idea that the “amylogenin” molecule of WO94/04693 is involved in starch synthesis. In further work (Langeveld, M. J. S et al. 2002 Plant Physiol, 129, pp 278-289) it is concluded that wheat and rice RGPs do not play a role in starch synthesis in a way similar to the functioning of glycogenin as a primer for glycogen synthesis. It is reported that RGP1 and RGP2 proteins in wheat and rice have different functions to glycogenin.

Lightner et al. US 2002/0001843 described fragments of putative “corn, wheat, and rice glycogenin and water stress proteins.” Lightner et al. did not demonstrate the functionality of the fragments, but only their sequence homology to glycogenin from animals. To date, therefore, no one has identified and demonstrated a functional protein for starch initiation or starch priming in plants.

Purified starch is used in numerous food and industrial applications and is the major source of carbohydrates in the human diet. Typically, starch is mixed with water and cooked to form a thickening agent or gel. Of central importance are the temperature at which the starch cooks, the viscosity that the agent or gel reaches, and the stability of the gel viscosity over time. The physical properties of unmodified starch limit its usefulness in many applications. As a result, considerable effort and expenditure is allocated to chemically modify starch (i.e. cross-linking and substitution) in order to overcome the numerous limitations of unmodified starch and to expand industrial usefulness. Modified starches can be used in foods, paper, textiles, and adhesives.

### 3 SUMMARY OF THE INVENTION

The invention provides isolated nucleic acids which encompass plant glycogenin-like nucleic acid molecules, plant glycogenin-like gene products (including, but not limited to, transcriptional products such as mRNAs, antisense and ribozyme molecules, and translational products such as plant glycogenin-like proteins, polypeptides, peptides and fusion proteins related thereto), antibodies to plant glycogenin-like gene products, plant glycogenin-like regulatory regions, vectors and expression vectors with plant glycogenin-like genes, cells, plants and plant parts with plant glycogenin-like genes, modified starch from such plants and the use of the foregoing to improve agronomically valuable plants, including but not limited to maize, wheat, barley and potato.

The invention is based upon the identification of a protein responsible for initiation of starch synthesis in plants, which despite continued efforts over the last few years, no one had yet successfully identified. In particular, the inventors have discovered nucleic acid molecules from Arabidopsis which have sequences that are homologous to the known glycogenin genes of yeast and human. Analysis of one of this nucleic acid molecule indicates that it contains a sequence encoding a transit peptide for plastid localization of the gene product, consistent with a role in starch synthesis, referred to herein as plant glycogenin-like starch initiation protein (PGSIP). Glycogenin-like genes from other plant species have been identified by analysis of sequence homology with the Arabidopsis sequences. The genes of the invention do not show homology to the amylogenin sequences or starch sequences of the prior art.

Modulation of the initiation of starch synthesis allows various aspects of the biosynthetic process to be regulated. By altering aspects of the biosynthesis process such as temporal and spatial specificity, yield and storage, the carbohydrate profile of the plant may be altered in magnitude and directions that may be more favorable for nutritional or industrial uses.

The present invention provides for an isolated nucleic acid molecule that i) comprises a nucleotide sequence which encodes a polypeptide comprising the amino acid sequence of SEQ ID NO: 3, or a fragment thereof; ii) comprises a nucleotide sequence at least 40% identical to SEQ ID NOs: 1 or 2, or a complement thereof as determined using the BESTFIT or GAP programs with a gap weight of 50 and a length weight of 3; or iii) hybridizes to a nucleic acid molecule consisting of SEQ ID NOs: 1 or 2 under low stringency conditions of hybridization consisting of washing at 60° C. for 2×15 minutes at 2×SSC (Sodium Citrate), 0.5% SDS, or a complement thereof. The present invention also provides for an isolated nucleic acid molecule of the invention comprising SEQ ID NOs: 1 or 2 or a complement thereof. In an embodiment of the invention, an isolated nucleic acid molecule comprises a nucleotide sequence selected from the group consisting of nucleotide residues 377 to 423, 516 to 592, 1039 to 1655, 1762 to 2536, and 2991 to 3264 of SEQ ID NO: 1.

Another embodiment of the invention encompasses an isolated nucleic acid molecule of the invention that i) comprises a nucleotide sequence which encodes a polypeptide comprising the amino acid sequence of SEQ ID NO: 11, or a fragment thereof, ii) comprises a nucleotide sequence at least 70% identical to SEQ ID NO: 10, or a complement thereof as determined using the BESTFIT or GAP programs with a gap weight of 50 and a length weight of 3, wherein the nucleotide sequence does not encode an amino acid of SEQ ID NO: 35; or iii) hybridizes to a nucleic acid molecule consisting of SEQ ID NO: 10 under stringent conditions of hybridization, or a complement thereof, wherein the sequence does not encode an amino acid of SEQ ID NO: 35. In a related embodiment, the isolated nucleic acid molecule of the invention comprises SEQ ID NO: 10 or a complement thereof. In another related embodiment an isolated nucleic acid molecule of the invention comprises the amino acid sequence that is at least 98% identical to SEQ ID NO: 9 as determined using the BESTFIT or GAP programs with a gap weight of 12 and a length weight of 4. The invention also encompasses an isolated nucleic acid molecule that comprises the nucleotide sequence of SEQ ID NO: 8 or a complement thereof.

In an embodiment of the invention, an isolated nucleic acid molecule of the invention i) comprises a nucleotide sequence which encodes a polypeptide comprising the amino acid sequence of SEQ ID NOs: 7, 13, 15, 17, 19, 21, 22, 24, 26, 28, 30, 32, 34, or a fragment thereof; ii) comprises a nucleotide sequence at least 70% identical to SEQ ID NOs: 4, 5, 6, 12, 14, 16, 18, 20, 23, 25, 27, 29, 31, 33, or a complement thereof as determined using the BESTFIT or GAP programs with a gap weight of 50 and a length weight of 3; or iii) hybridizes to a nucleic acid molecule consisting of SEQ ID NOs: 4, 5, 6, 12, 14, 16, 18, 20, 23, 25, 27, 29, 31, 33 under stringent conditions of hybridization, or a complement thereof. In a related embodiment, the isolated nucleic acid molecule of the invention comprises SEQ ID NOs: 4, 5, 6, 12, 14, 16, 18, 20, 23, 25, 27, 29, 31, 33, or a complement thereof. In another embodiment of the invention, a fragment of the isolated nucleic acid molecule of the invention comprises at least 40, 60, 80, 100 or 150 contiguous nucleotides of the nucleic acid molecule. In yet another embodiment, the isolated nucleic acid molecule of the invention comprises the nucleotide sequence of nucleotides 1-195 of SEQ ID NO: 2, or a complement thereof.

According to one aspect of the invention, an isolated polypeptide of the invention comprises the amino acid sequence of amino acid residues 1-65 of SEQ ID NO: 3, or a fragment thereof. In a related aspect, an isolated polypeptide comprises i) an amino acid sequence that is at least 70% identical to SEQ ID NO: 3 or a fragment thereof as determined using the BESTFIT or GAP programs with a gap weight of 12 and a length weight of 4; ii) an amino acid sequence encoded by the nucleic acid molecule of the invention; or iii) an amino acid sequence of SEQ ID NO: 3.

An embodiment of the invention encompasses an isolated polypeptide of the invention that comprises i) an amino acid sequence at least 70% identical to SEQ ID NO: 11 as determined using the BESTFIT or GAP programs with a gap weight of 12 and a length weight of 4, or a fragment thereof; ii) an amino acid sequence encoded by the nucleic acid molecule of of the invention; or iii) an amino acid sequence of SEQ ID NO: 11.

In another embodiment of the invention, an isolated polypeptide of the invention comprises i) an amino acid sequence that is at least 98% identical to SEQ ID NO: 9 as determined using the BESTFIT or GAP programs with a gap weight of 12 and a length weight of 4; iii) an amino acid sequence encoded by the nucleic acid molecule of SEQ ID NO: 8, or a complement thereof; or v) an amino acid sequence of SEQ ID NO: 9, or a fragment thereof.

The invention further provides for an isolated polypeptide that comprises i) an amino acid sequence that is at least 70% identical to SEQ ID NOs: 7, 13, 15, 17, 19, 21, 22, 24, 26, 28, 30, 32, 34, or a fragment thereof as determined using the BESTFIT or GAP programs with a gap weight of 12 and a length weight of 4; ii) an amino acid sequence encoded by the nucleic acid molecule of the invention; iii) an amino acid sequence of SEQ ID NOs: 7, 13, 15, 17, 19, 21, 22, 24, 26, 28, 30, 32, 34. In an embodiment of the invention, a fragment of a polypeptide of the invention comprises at least 5 amino acid residues, wherein said fragment is a portion of the polypeptide encoded by a nucleic acid molecule selected from the group consisting of exon I, exon II, exon III, exon IV and exon V of SEQ ID NO: 1.

Another embodiment of the invention encompasses the polypeptide of SEQ ID: 3, 7, 9, 11, 13, 15, 17, 19, 21, 22, 24, 26, 28, 30, 32, 34 further comprising one or more conservative amino acid substitution. In yet another embodiment, the invention provides for a fusion protein comprising the amino acid sequence of the invention and a heterologous protein.

The invention provides for an isolated polypeptide fragment or immunogenic fragment that comprises at least 5, 8, 10, 15, 20, 25, 30 or 35 consecutive amino acids of the polypeptide. The invention further provides for an antibody that immunospecifically binds to a polypeptide of the invention.

In one embodiment the invention encompasses a method for making a polypeptide of any one of the invention, comprising the steps of a) culturing a cell comprising a recombinant polynucleotide encoding the polypeptide of the invention under conditions that allow said polypeptide to be expressed by said cell; and b) recovering the expressed polypeptide.

According to another aspect of the invention, a complex comprising a polypeptide encoded by a nucleic acid molecule of the invention and a starch molecule. In one embodiment of the complex of the invention, the starch molecule comprises from 1 to 700 glucose units. In another embodiment of the complex of the invention the starch molecule comprises branching chains of glucose polysaccharides.

According to yet another aspect of the invention, a vector comprises a nucleic acid molecule of the invention. Alternatively, an expression vector comprises a nucleic acid molecule of the invention and at least one regulatory region operably linked to the nucleic acid molecule.

In another embodiment, the expression vector of the invention comprises a regulatory region confers chemically-inducible, dark-inducible, developmentally regulated, developmental-stage specific, wound-induced, environmental factor-regulated, organ-specific, cell-specific, and/or tissue-specific expression of the nucleic acid molecule or constitutive expression of the nucleic acid molecule of the invention. In yet another embodiment, the expression vector of the invention, comprises a regulatory region selected from the group consisting of a 35S CaMV promoter, a rice actin promoter, a patatin promoter, and a high molecular weight glutenin gene of wheat. In another embodiment, an expression vector of the invention comprises the antisense sequence of a nucleic acid molecules of the invention, wherein the antisense sequence is operably linked to at least one regulatory region.

The invention also provides for a genetically-engineered cell which comprises a nucleic acid molecule of the invention. In one embodiment, a cell comprises the expression vector of the invention comprising a nucleic acid molecule of the invention and at least one regulatory region operably linked to the nucleic acid molecule. In another embodiment, a cell comprises the expression vector of the invention comprising the antisense sequence of a nucleic acid molecules of the invention, wherein the antisense sequence is operably linked to at least one regulatory region.

In another embodiment of the invention, a cell comprises the expression vector comprising the antisense sequence of a nucleic acid molecules of the invention, wherein the antisense sequence is operably linked to at least one regulatory region

Yet another aspect of the invention provides for a genetically-engineered plant comprising the isolated nucleic acid molecule of the invention. The invention also provides for the genetically-engineered plant comprising the isolated nucleic acid molecule of the invention and progeny thereof, further comprising a transgene encoding an antisense nucleotide sequence. The invention also provides for the genetically-engineered plant comprising the isolated nucleic acid molecule of the invention, further comprising an RNA interference construct.

An embodiment of the invention encompasses a cell comprising an a 35SCaMV constitutive promoter operably linked to a nucleic acid molecule of SEQ ID NO:2 or a rice actin promoter operably linked to an RNA interference construct comprising fragments of a nucleic acid molecule of SEQ ID NO:2.

Another embodiment of the invention provides for a method of altering starch synthesis in a plant comprising, introducing into a plant an expression vector of the invention, such that starch synthesis is altered relative to a plant without the expression vector. Yet another embodiment of the invention provides for a method of altering starch synthesis in a plant comprising, introducing into a plant at least an expression vector comprising the antisense sequence of a nucleic acid molecules of the invention, wherein the antisense sequence is operably linked to at least one regulatory region, such that starch synthesis is altered in comparison to a plant without the expression vector.

In an embodiment of the invention, a method of altering starch granules in a plant comprises introducing into a plant at least an expression vector comprising a nucleic acid molecule of the invention and at least one regulatory region operably linked to the nucleic acid molecule, such that the starch granules are altered in comparison to a plant without the expression vector.

In another embodiment of the invention, a method of altering starch granules in a plant comprises introducing into a plant at least an expression vector of claim 30, such that the starch granules are altered in comparison to a plant without the expression vector.

In yet another embodiment of the invention, the method of altering starch granules in a plant comprises introducing into a plant at least an expression vector comprising a nucleic acid molecule of the invention and at least one regulatory region operably linked to the nucleic acid molecule, such that the starch granules are absent from leaves of the plant comprising at least an expression vector.

In a preferred embodiment of the invention, a plant part comprises a nucleic acid molecule of the invention resulting in an alteration in starch synthesis. In another preferred embodiment the plant part is a tuber, seed, or leaf.

The invention also provides for the modified starch obtained from the plant parts of the invention, wherein the modification is selected from the group consisting of a ratio of amylose to amylopectin, amylose content, size of starch granules, quantity of size of starch granules, a ratio of small to large starch granules, and rheological properties of the starch as measured using viscometric analysis.

### 3.1 SEQUENCE IDENTIFIERS

The present invention will now be illustrated by way of non-limiting examples of biological sequences in which:

SEQ ID NO: 1 shows the genomic sequence of a starch primer gene isolated from Arabidopsis thaliana referred to herein as plant glycogenin-like starch initiation protein (PGSIP), at3g18660, GenBank Accession No. NM_{—}112752. The gene includes part of the promoter region, where the putative TATA and CAAT box are located at nucleotides 424-428 and 373-376 respectively. The exons are located at nucleotides 377 to 423, 516 to 592, 1039 to 1655, 1762 to 2536, and 2991 to 3264.

SEQ ID NO: 2 shows the deduced cDNA sequence of Arabidopsis thaliana PGSIP with protein translation. The transit peptide is located at nucleotides 1-195.

SEQ ID NO:3 shows the amino acid sequence representing the Arabidopsis thaliana PGSIP protein. The predicted transit peptide is located at amino acid residues 1-65.

SEQ ID NO:4 shows the nucleotide sequence of the maize EST of GenBank Accession No. BF729544 with homology to the Arabidopsis thaliana PGSIP gene. The nucleotide sequence with homology to the Arabidopsis thaliana PGSIP gene is located at nucleotides 1-557.

SEQ ID NO:5 shows the nucleotide sequence of the maize EST BG837930 showing homology to Arabidopsis thaliana PGSIP gene. The nucleotide sequence with homology to the Arabidopsis thaliana PGSIP gene is located at nucleotides 1-726.

SEQ ID NO:6 shows the deduced cDNA of the Arabidopsis glycogenin-like gene (at1g77130) with protein translation. The protein sequence with homology to a small region (amino acid residues 1023-1146) of dull1 gene from maize (064923).

SEQ ID NO:7 shows the amino acid sequence of at1g77130.

SEQ ID NO:8 shows the deduced cDNA of the Arabidopsis glycogenin-like gene (at1g08990) GenBank Accession No. NM_{—}100770.

SEQ ID NO:9 shows the amino acid sequence of at1g08990.

SEQ ID NO:10 shows the deduced cDNA of the Arabidopsis glycogenin-like gene (at1g54940) GenBank Accession No. NM_{—}104367.

SEQ ID NO:11 shows the amino acid sequence of at1g54940.

SEQ ID NO: 12 shows the deduced cDNA of the Arabidopsis glycogenin-like gene (at4 g33330) GenBank Accession No. NM_{—}119487.

SEQ ID NO:13 shows the amino acid sequence of at4 g33330.

SEQ ID NO: 14 shows the deduced cDNA of the Arabidopsis glycogenin-like gene (at4 g33340) GenBank Accession No. NM_{—}119488.

SEQ ID NO:15 shows the amino acid sequence of at4 g33340.

Sequence ID No.16 shows the nucleotide sequence of Barley EST Seq1.

SEQ ID NO:17 shows the amino acid sequence of Barley EST Seq1.

SEQ ID NO:18 shows the nucleotide sequence of Barley EST Seq2.

SEQ ID NO:19 shows the amino acid sequence of Barley EST Seq2.

SEQ ID NO:20 shows the nucleotide sequence of a wheat EST.

SEQ ID NO:21 shows the first half of the amino acid sequence of the wheat EST.

SEQ ID NO:22 shows the second half of the amino acid sequence of the wheat EST.

SEQ ID NO:23 shows the deduced cDNA of the Arabidopsis gene EMBL:AY062695 GenBank Accession No. AY062695 with homology to the Arabidopsis PGSIP gene.

SEQ ID NO:24 shows the amino acid sequence of EMBL:AY062695.

SEQ ID NO:25 shows the deduced cDNA of the Rice gene SPTrEMBL:Q94HG3 GenBank Accession No. AC079633 with homology to the Arabidopsis PGSIP gene.

SEQ ID NO:26 shows the amino acid sequence of SPTrEMBL:Q94HG3.

SEQ ID NO:27 shows the nucleotide sequence of Maize EST Seq1.

SEQ ID NO:28 shows the amino acid sequence of Maize EST Seq1.

SEQ ID NO:29 shows the nucleotide sequence of Maize EST Seq2.

SEQ ID NO:30 shows the amino acid sequence of Maize EST Seq2.

SEQ ID NO:31 shows the nucleotide sequence of Maize EST Seq3.

SEQ ID NO:32 shows the amino acid sequence of Maize EST Seq3.

SEQ ID NO:33 shows the nucleotide sequence of Maize EST Seq4.

SEQ ID NO: 34 shows the amino acid sequence of Maize EST Seq4.

SEQ ID NO: 35 shows an amino acid sequence as a result of a conceptual translation of a portion of a genomic clone from Arabidopsis thaliana as disclosed in U.S. Patent Application No. 2002/0001843.

### 4 BRIEF DESCRIPTION OF THE FIGURES

FIG. 1 shows the plasmid containing the Arabidopsis thaliana plant glycogenin-like starch initiation protein (PGSIP) gene.

FIG. 2 shows the plasmid map for pTPYES.

FIG. 3 shows the plasmid map for pNTPYES

FIG. 4A shows a genomic region containing AT3g18660 (PGSIP); 4B shows a non-radioactive southern blot of Arabidopsis, wheat and maize genomic DNA probed with C-terminus AT3g18660 cDNA high stringency conditions. N-NcoI, A-AvaI, C-ClaI.

FIG. 5A shows a non-radioactive southern blot of Arabidopsis, wheat and maize genomic DNA probed with N-terminal ATg18660 (PGSIP) cDNA fragment under low Stringent conditions. N-NcoI, A-AvaI, C-ClaI. Lane M is a marker, lane 1 is AT (EcoRI), lane 2 is AT (XhoI), lane 3 is AT (EcoRV), lane 4 is wheat (EcoRI), lane 5 is wheat (XhoI), lane 6 is wheat EcoRV), lane 7 is maize (Ecor RI), lane 8 is maize (XhoI), and lane 9 is maize (EcoRV); 5B shows a non-radioactive southern blot of Arabidopsis, wheat and maize genomic DNA probed with C-terminal ATg18660 (PGSIP) cDNA fragment under low Stringent conditions. N-NcoI, A-AvaI, C-ClaI. Lane M is a marker, lane 1 is AT (EcoRI), lane 2 is AT (XhoI), lane 3 is AT (EcoRV), lane 4 is wheat (EcoRI), lane 5 is wheat (XhoI), lane 6 is wheat EcoRV), lane 7 is maize (Ecor RI), lane 8 is maize (XhoI), and lane 9 is maize (EcoRV).

FIG. 6 shows the cloning strategy and plasmid maps for the production of the PGSIP RNAi construct pCL76 SCV.

FIG. 7 shows the plasmid map for pCL68 SCV. (Sense expression construct) containing the AT3g18660 (PGSIP) cDNA.

FIG. 8 shows the plasmid map for pCL76 SCV.(RNAi construct) containing fragments of the AT3g18660 (PGSIP) cDNA.

FIG. 9 shows the plasmid map for pMC177 (Sense expression construct) containing the AT3g18660 (PGSIP) under rice actin promoter used in barley and Arabidopsis transformation.

FIG. 10 shows the plasmid map for pMC176 (RNAi construct) containing the AT3g18660 (PGSIP) under rice actin promoter used in barley and Arabidopsis transformation.

FIG. 11A shows the results of iodine staining of leaves of barley which was shown to be PCR positive for the (pCL76 SCV) RNAi PGSIP constructs. Starch grains are absent; 11B shows the results of iodine staining of leaves of barley which was shown to be PCR negative for the (pCL76 SCV) RNAi PGSIP constructs. Starch grains are visible.

### 5 DETAILED DESCRIPTION OF THE INVENTION

The invention relates to a family of plant glycogenin-like genes, also referred to as starch primer genes. In various embodiments, the invention provides plant glycogenin-like nucleic acid molecules including, but not limited to, plant glycogenin-like genes; plant glycogenin-like regulatory regions; plant glycogenin-like promoters; and vectors incorporating sequences encoding plant glycogenin-like nucleic acid molecules of the invention. Also provided are plant glycogenin-like gene products, including, but not limited to, transcriptional products such as mRNAs, antisense and ribozyme molecules, and translational products such as the plant glycogenin-like protein, polypeptides, peptides and fusion proteins related thereto; genetically engineered host cells that contain any of the foregoing nucleic acid molecules and/or coding sequences or compliments, variants, or fragments thereof operatively associated with a regulatory element that directs the expression of the gene and/or coding sequences in the host cell; genetically-engineered plants derived from host cells; modified starch and starch granules produced by genetically-engineered host cells and plants; and the use of the foregoing to improve agronomically valuable plants.

In the context of the present invention, a “starch primer” used interchangeably with “plant glycogenin-like protein” includes any protein which is capable of initiating starch production in a plant. By definition, the plant glycogenin-like protein will be of plant origin. Preferred fragments of plant glycogenin-like proteins are those which retain the ability to initiate starch synthesis.

For purposes of clarity, and not by way of limitation, the invention is described in the subsections below in terms of (a) plant glycogenin-like nucleic acid molecules; (b) plant glycogenin-like gene products; (c) transgenic plants that ectopically express plant glycogenin-like protein; (d); transgenic plants in which endogenous plant glycogenin-like protein expression is suppressed; (e) starch characterized by altered structure and physical properties produced by the methods of the invention.

### 5.1 Plant Glycogenin-Like Nucleic Acids

The nucleic acid molecules of the invention may be DNA, RNA and comprises the nucleotide sequences of a plant glycogenin-like gene, or fragments or variants thereof. A polynucleotide is intended to include DNA molecules (e.g., cDNA, genomic DNA), RNA molecules (e.g., hnRNA, pre-mRNA, mRNA, double-stranded RNA), and DNA or RNA analogs generated using nucleotide analogs. The polynucleotide can be single-stranded or double-stranded.

The nucleic acid molecules are characterized by their homology to known glycogen primer (glycogenin) genes, such as those from yeast (Glg1 and Glg2), human (any isoform), C. elegans, rat or rabbit, or plant glycogenin-like gene such as those defined herein. A preferred nucleic acid molecule of this embodiment is one that encodes the amino acid sequence of SEQ ID NO: 2, or a fragment or variant thereof, or a nucleic acid molecule comprising a sequence substantially similar to SEQ ID NO: 2. In a most preferred embodiment, the nucleic acid molecule comprises the nucleotide sequence shown in SEQ ID NO: 1, or a fragment or variant thereof, or a sequence substantially similar to SEQ ID NO: 1. The variants may be an allelic variants. Allelic variants being multiple forms of a particular gene or protein encoded by a particular gene. Fragments of a plant glycogenin-like gene may include regulatory elements of the gene such as promoters, enhancers, transcription factor binding sites, and/or segments of a coding sequence for example, a conserved domain, exon, or transit peptide.

In a preferred embodiment, the nucleic acid molecules of the invention are comprised of full length sequences in that they encode an entire plant glycogenin-like protein as it occurs in nature. Examples of such sequences include SEQ ID NOs: 1, 2, 6, 8, 10, 12, and 14. The corresponding amino acid sequences of full length glycogenin-like proteins are SEQ ID NOs: 3, 7, 9, 11, 13, and 15. In an alternative embodiment, the nucleic acid molecules of the invention comprise a nucleotide sequence of SEQ ID NOs: 1, 2, 4, 5, 6, 8, 10, 12, 14, 16, 18, 20, 23, 25, 27, 29, 31, or 33.

The nucleic acid molecules and their variants can be identified by several approaches including but not limited to analysis of sequence similarity and hybridization assays.

In the context of the present invention the term “substantially homologous,” “substantially identical,” or “substantial similarity,” when used herein with respect to sequences of nucleic acid molecules, means that the sequence has either at least 45% sequence identity with the reference sequence, preferably 50% sequence identity, more preferably at least 60%, 70%, 80%, 90% and most preferably at least 95% sequence identity with said sequences, in some cases the sequence identity may be 98% or more preferably 99%, or above, or the term means that the nucleic acid molecule is either is capable of hybridizing to the complement of the nucleic acid molecule having the reference sequence under stringent conditions.

“% identity”, as known in the art, is a measure of the relationship between two polynucleotides or two polypeptides, as determined by comparing their sequences. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology.

For example, sequences can be aligned with the software clustalw under Unix which generates a file with a “.aln” extension, this file can then be imported into the Bioedit program (Hall, T. A. 1999. BioEdit: a user-friendly biological sequence alignment editor and analysis program for Windows 95/98/NT. Nucl. Acids. Symp. Ser. 41:95-98) which opens the .aln file. In the Bioedit window, one can choose individual sequences (two at a time) and alignment them. This method allows for comparison of the entire sequences.

Methods for comparing the identity of two or more sequences are well known in the art. Thus for instance, programs available in the Wisconsin Sequence Analysis Package, version 9.1 (Devereux J et al, Nucleic Acids Res. 12:387-395, 1984, available from Genetics Computer Group, Maidson, Wisconsin, USA). The determination of percent identity between two sequences can be accomplished using a mathematical algorithm. For example, the programs BESTFIT and GAP, may be used to determine the % identity between two polynucleotides and the % identity between two polypeptide sequences. BESTFIT uses the “local homology” algorithm of Smith and Waterman (Advances in Applied Mathematics, 2:482-489, 1981) and finds the best single region of similarity between two sequences. BESTFIT is more suited to comparing two polynucleotide or two polypeptide sequences which are dissimilar in length, the program assuming that the shorter sequence represents a portion of the longer. In comparison, GAP aligns two sequences finding a “maximum similarity” according to the algorithm of Neddleman and Wunsch (J. Mol. Biol. 48:443-354, 1970). GAP is more suited to comparing sequences which are approximately the same length and an alignment is expected over the entire length. Preferably the parameters “Gap Weight” and “Length Weight” used in each program are 50 and 3 for polynucleotides and 12 and 4 for polypeptides, respectively. Preferably % identities and similarities are determined when the two sequences being compared are optimally aligned.

Other programs for determining identity and/or similarity between sequences are also known in the art, for instance the BLAST family of programs (Karlin & Altschul, 1990, Proc. Natl. Acad. Sci. USA 87:2264-2268, modified as in Karlin & Altschul, 1993, Proc. Natl. Acad. Sci. USA 90:5873-5877, available from the National Center for Biotechnology Information (NCB), Bethesda, Md., USA and accessible through the home page of the NCBI at www.ncbi.nlm.nih.gov). These programs exemplify a preferred, non-limiting example of a mathematical algorithm utilized for the comparison of two sequences. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a program for determining identity and/or similarity between sequences known in the art is FASTA (Pearson W. R. and Lipman D. J., Proc. Nat. Acac. Sci., USA, 85:2444-2448, 1988, available as part of the Wisconsin Sequence Analysis Package). Preferably the BLOSUM62 amino acid substitution matrix (Henikoff S. and Henikoff J. G., Proc. Nat. Acad. Sci., USA, 89:10915-10919, 1992) is used in polypeptide sequence comparisons including where nucleotide sequences are first translated into amino acid sequences before comparison.

Yet another non-limiting example of a program known in the art for determining identity and/or similarity between amino acid sequences is SeqWeb Software (a web-based interface to the GCG Wisconsin Package: Gap program) which is utilized with the default algorithm and parameter settings of the program: blosum 62, gap weight 8, length weight 2.

The percent identity between two sequences can be determined using techniques similar to those described above, with or without allowing gaps. In calculating percent identity, typically exact matches are counted.

Preferably the program BESTFIT is used to determine the % identity of a query polynucleotide or a polypeptide sequence with respect to a polynucleotide or a polypeptide sequence of the present invention, the query and the reference sequence being optimally aligned and the parameters of the program set at the default value.

Alternatively, variants and fragments of the nucleic acid molecules of the invention can be identified by hybridization to SEQ ID NOs: 1, 2, 4-6, 8, 10, 12, 14, 16, 18, 20, 23, 25, 27, 29, 31, or 33. In the context of the present invention “stringent conditions” are defined as those given in Martin et al (EMBO J. 4:1625-1630 (1985)) and Davies et al (Methods in Molecular Biology Vol 28: Protocols for nucleic acid analysis by non-radioactive probes, Isaac, P. G. (ed) pp 9-15, Humana Press Inc., Totowa N.J, USA)).

The conditions under which hybridization and/or washing can be carried out can range from 42° C. to 68° C. and the washing buffer can comprise from 0.1×SSC, 0.5% SDS to 6×SSC, 0.5% SDS. Typically, hybridization can be carried out overnight at 65° C. (high stringency conditions), 60° C. (medium stringency conditions), or 55° C. (low stringency conditions). The filters can be washed for 2×15 minutes with 0.1×SSC, 0.5% SDS at 65° C. (high stringency washing). The filters were washed for 2×15 minutes with 0.1×SSC, 0.5% SDS at 63° C. (medium stringency washing). For low stringency washing, the filters were washed at 60° C. for 2×15 minutes at 2×SSC, 0.5% SDS.

In instances wherein the nucleic acid molecules are oligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may act as plant glycogenin-like gene antisense molecules, useful, for example, in plant glycogenin-like gene regulation and/or as antisense primers in amplification reactions of plant glycogenin-like gene and/or nucleic acid molecules. Further, such nucleic acid molecules may be used as part of ribozyme and/or triple helix sequences, also useful for plant glycogenin-like gene regulation. Still further, such molecules may be used as components in probing methods whereby the presence of a plant glycogenin-like allele may be detected.

In one embodiment, a nucleic acid molecule of the invention may be used to identify other plant glycogenin-like genes by identifying homologs. This procedure may be performed using standard techniques known in the art, for example screening of a cDNA library by probing; amplification of candidate nucleic acid molecules; complementation analysis, and yeast two-hybrid system (Fields and Song Nature 340 245-246 (1989); Green and Hannah Plant Cell 10 1295-1306 (1998)).

The invention also includes nucleic acid molecules, preferably DNA molecules, that are amplified using the polymerase chain reaction and that encode a gene product functionally equivalent to a plant glycogenin-like gene product.

In another embodiment of the invention, nucleic acid molecules which hybridize under stringent conditions to the nucleic acid molecules comprising a plant glycogenin-like gene and its complement are used in altering starch synthesis in a plant. Such nucleic acid molecules may hybridize to any part of a plant glycogenin-like gene, including the regulatory elements. Preferred nucleic acid molecules are those which hybridize under stringent conditions to a nucleic acid molecule comprising the nucleotide sequence encoding the amino acid sequence of SEQ ID NO: 2, and/or a nucleotide sequence of any one of SEQ ID NOs: 1, 2, 4-6, 8, 10, 12, 14, 16, 18, 20, 23, 25, 27, 29, 31, or 33 or their complement sequences. Preferably the nucleic acid molecule which hybridizes under stringent conditions to a nucleic acid molecule comprising the sequence of a plant glycogenin-like gene or its complement are complementary to the nucleic acid molecule to which they hybridize.

In another embodiment of the invention, nucleic acid molecules which hybridize under stringent conditions to the nucleic acid molecules of SEQ ID NOs: 1, 2, 4-6, 8, 10, 12, 14, 16, 18, 20, 23, 25, 27, 29, 31, or 33 hybridize over the full length of the sequences of the nucleic acid molecules.

Alternatively, nucleic acid molecules of the invention or their expression products may be used in screening for agents which alter the activity of a plant glycogenin-like protein of a plant. Such a screen will typically comprise contacting a putative agent with a nucleic acid molecule of the invention or expression product thereof and monitoring the reaction there between. The reaction may be monitored by expression of a reporter gene operably linked to a nucleic acid molecule of the invention, or by binding assays which will be known to persons skilled in the art.

Fragments of a plant glycogenin-like nucleic acid molecule of the invention preferably comprise or consist of at least 40 continuous or consecutive nucleotides of the plant glycogenin-like nucleic acid molecule of the invention, more preferably at least 60 nucleotides, at least 80 nucleotides, or most preferably at least 100 or 150 nucleotides in length. Fragments of a plant glycogenin-like nucleic acid molecule of the invention encompassed by the invention may include elements involved in regulating expression of the gene or may encode functional plant glycogenin-like proteins. Fragments of the nucleic acid molecules of the invention encompasses fragments of SEQ ID NOs: 1, 2, 4-6, 8, 10, 12, 14, 16, 18, 20, 23, 25, 27, 29, 31 and 33 as well as fragments of the variants of those sequences identified as defined above by percent homology or hybridization assay.

Examples of fragments encompassed by the invention include exons of the PGSIP gene. SEQ ID NO: 1 indicates exon and intron boundaries of the plant glycogenin-like gene PGSIP. Nucleic acid molecules comprising PGSIP exon and intron sequences are encompassed by the present invention. In one embodiment, five exons are included (SEQ ID NO: 1; GenBank Accession No. NM_{—}112752). PGSIP exon 1 encompasses nucleotides 377 to 423 of SEQ ID NO: 1. of the sequence shown in SEQ ID NO: 1; exon 2 encompasses nucleotides 516 to 592 of the sequence shown in SEQ ID NO:1; exon 3 encompasses nucleotides 1039 to 1655 of the sequence shown in SEQ ID NO: 1; exon 4 encompasses nucleotides 1762 to 2536 of the sequence shown in SEQ ID NO: 1; exon 5 encompasses nucleotides 2991 to 3264 of the sequence shown in SEQ ID NO: 1.

Further, a plant glycogenin-like nucleic acid molecule of the invention can comprise two or more of any above-described sequences, or variants thereof, linked together to form a larger subsequence.

The nucleic acid molecules of the invention can comprise or consist of an EST sequence. The EST nucleic acid molecules of the invention can be used as probes for cloning corresponding full length genes. For example, the barley EST of SEQ ID NO: 16 can be utilized as a probe in identifying and cloning the full length Barley homolog of the Arabidopsis PGSIP gene. The EST nucleic acid molecules of the invention may be used as sequence probes in connection with computer software to search databases, such as GenBank for homologous sequences. Alternatively, the EST nucleic acid molecules can be used as probes in hybridization reactions as described herein. The EST nucleic acid molecules of the invention can also be used as molecular markers to map chromosome regions.

In certain embodiments, the plant glycogenin-like nucleic acid molecules and polypeptides do not include sequences consisting of those sequences known in the art. For example, in one embodiment, the plant glycogenin-like nucleic acid molecules do not include EST sequences.

In other embodiments, the plant glycogenin-like nucleic acid molecules of the invention, encode polypeptides that function as plant glycogenin-like proteins. The functionality of such nucleic acid molecules can be assessed using the yeast hybrid complementation assay as described herein in Example 3. Alternatively, the functionality of such nucleic acid molecules can be assessed using a complementation assay in Arabidopsis as described in this section.

An isolated nucleic acid molecule encoding a variant protein can be created by introducing one or more nucleotide substitutions, additions or deletions into the plant glycogenin-like nucleic acid molecule, such that one or more amino acid substitutions, additions or deletions are introduced into the encoded protein. Mutations can be introduced by standard techniques, such as, ethyl methane sulfonate, X-rays, gamma rays, T-DNA mutagenesis, or site-directed mutagenesis, PCR-mediated mutagenesis. Briefly, PCR primers are designed that delete the trinucleotide codon of the amino acid to be changed and replace it with the trinucleotide codon of the amino acid to be included. This primer is used in the PCR amplification of DNA encoding the protein of interest. This fragment is then isolated and inserted into the full length cDNA encoding the protein of interest and expressed recombinantly.

An isolated nucleic acid molecule encoding a variant protein can be created by any of the methods described in section 5.1. Either conservative or non-conservative amino acid substitutions can be made at one or more amino acid residues. Both conservative and non-conservative substitutions can be made. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (See, for example, Biochemistry, 4th ed., Ed. by L. Stryer, W H Freeman and Co.: 1995).

Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The invention also encompasses (a) DNA vectors that contain any of the foregoing nucleic acids and/or coding sequences (i.e. fragments and variants) and/or their complements (i.e., antisense molecules); (b) DNA expression vectors that contain any of the foregoing nucleic acids and/or coding sequences operatively associated with a regulatory region that directs the expression of the nucleic acids and/or coding sequences; and (c) genetically engineered host cells that contain any of the foregoing nucleic acids and/or coding sequences operatively associated with a regulatory region that directs the expression of the gene and/or coding sequences in the host cell. As used herein, regulatory region include, but are not limited to, inducible and non-inducible genetic elements known to those skilled in the art that drive and regulate expression of a nucleic acid. The nucleic acid molecules of the invention may be under the control of a promoter, enhancer, operator, cis-acting sequences, or trans-acting factors, or other regulatory sequence. The nucleic acid molecules encoding regulatory regions of the invention may also be functional fragments of a promoter or enhancer. The nucleic acid molecules encoding a regulatory region is preferably one which will target expression to desired cells, tissues, or developmental stages.

Examples of highly suitable nucleic acid molecules encoding regulatory regions are endosperm specific promoters, such as that of the high molecular weight glutenin (HMWG) gene of wheat, prolamin, or ITR1, or other suitable promoters available to the skilled person such as gliadin, branching enzyme, ADFG pyrophosphorylase, patatin, starch synthase, rice actin, and actin, for example.

Other suitable promoters include the stem organ specific promoter gSPO-A, the seed specific promoters Napin, KTI 1, 2, & 3, beta-conglycinin, beta-phaseolin, heliathin, phytohemaglutinin, legumin, zein, lectin, leghemoglobin c3, AB13, PvAlf, SH-EP, EP-C1, 2S1, EM 1, and ROM2.

Constitutive promoters, such as CaMV promoters, including CaMV 35S and CaMV 19S may also be suitable. Other examples of constitutive promoters include Actin 1, Ubiquitin 1, and HMG2.

In addition, the regulatory region of the invention may be one which is environmental factor-regulated such as promoters that respond to heat, cold, mechanical stress, light, ultra-violet light, drought, salt and pathogen attack. The regulatory region of the invention may also be one which is a hormone-regulated promoter that induces gene expression in response to phytohormones at different stages of plant growth. Useful inducible promoters include, but are not limited to, the promoters of ribulose bisphosphate carboxylase (RUBISCO) genes, chlorophyll a/b binding protein (CAB) genes, heat shock genes, the defense responsive gene (e.g., phenylalanine ammonia lyase genes), wound induced genes (e.g., hydroxyproline rich cell wall protein genes), chemically-inducible genes (e.g., nitrate reductase genes, gluconase genes, chitinase genes, PR-1 genes etc.), dark-inducible genes (e.g., asparagine synthetase gene as described by U.S. Pat. No. 5,256,558), and developmental-stage specific genes (e.g., Shoot Meristemless gene, AB13 promoter and the 2S1 and Em 1 promoters for seed development (Devic et al.,1996, Plant Journal 9(2):205-215), and the kin1 and cor6.6 promoters for seed development (Wang et al., 1995, Plant Molecular Biology, 28(4):619-634). Examples of other inducible promoters and developmental-stage specific promoters can be found in Datla et al., in particular in Table 1 of that publication (Datla et al., 1997, Biotechnology annual review 3:269-296).

A vector of the invention may also contain a sequence encoding a transit peptide which can be fused in-frame such that it is expressed as a fusion protein.

Methods which are well known to those skilled in the art can be used to construct vectors and/or expression vectors containing plant glycogenin-like protein coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, and Ausubel et al., 1989. Alternatively, RNA capable of encoding plant glycogenin-like protein sequences may be chemically synthesized using, for example, synthesizers. See, for example, the techniques described in Gait, 1984, Oligonucleotide Synthesis, IRL Press, Oxford. In a preferred embodiment of the invention, the techniques described in section 6, example 6, and illustrated in FIG. 6 are used to construct a vector.

A variety of host-expression vector systems may be utilized to express the plant glycogenin-like gene products of the invention. Such host-expression systems represent vehicles by which the plant glycogenin-like gene products of interest may be produced and subsequently recovered and/or purified from the culture or plant (using purification methods well known to those skilled in the art), but also represent cells which may, when transformed or transfected with the appropriate nucleic acid molecules, exhibit the plant glycogenin-like protein of the invention in situ. These include but are not limited to microorganisms such as bacteria (e.g., E. coli, B. subtilis) transformed with recombinant bacteriophage DNA, plasmid DNA or cosmid DNA expression vectors containing plant glycogenin-like protein coding sequences; yeast (e.g., Saccharomyces, Pichia) transformed with recombinant yeast expression vectors containing the plant glycogenin-like protein coding sequences; insect cell systems infected with recombinant virus expression vectors (e.g., baculovirus) containing the plant glycogenin-like protein coding sequences; plant cell systems infected with recombinant virus expression vectors (e.g., cauliflower mosaic virus, CaMV; tobacco mosaic virus, TMV); plant cell systems transformed with recombinant plasmid expression vectors (e.g., Ti plasmid) containing plant glycogenin-like protein coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter; the cytomegalovirus promoter/enhancer; etc.). In a preferred embodiment of the invention, an expression vector comprising a plant glycogenin-like nucleic acid molecule operably linked to at least one suitable regulatory sequence is incorporated into a plant by one of the methods described in this section, section 5.3, 5.4 and 5.5 or in examples 7, 8, 9, and 12.

In bacterial systems, a number of expression vectors may be advantageously selected depending upon the use intended for the plant glycogenin-like protein being expressed. For example, when a large quantity of such a protein is to be produced, for the generation of antibodies or to screen peptide libraries, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the plant glycogenin-like coding sequence may be ligated individually into the vector in frame with the lac Z coding region so that a fusion protein is produced; pIN vectors (Inouye & Inouye, 1985, Nucleic Acids Res. 13:3101-9; Van Heeke & Schuster, 1989, J. Biol. Chem. 264:5503-9); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

In one such embodiment of a bacterial system, full length cDNA nucleic acid molecules are appended with in-frame Bam HI sites at the amino terminus and Eco RI sites at the carboxyl terminus using standard PCR methodologies (Innis et al., 1990, supra) and ligated into the pGEX-2TK vector (Pharmacia, Uppsala, Sweden). The resulting cDNA construct contains a kinase recognition site at the amino terminus for radioactive labeling and glutathione S-transferase sequences at the carboxyl terminus for affinity purification (Nilsson, et al., 1985, EMBO J. 4:1075; Zabeau and Stanley, 1982, EMBO J. 1: 1217).

The recombinant constructs of the present invention may include a selectable marker for propagation of the construct. For example, a construct to be propagated in bacteria preferably contains an antibiotic resistance gene, such as one that confers resistance to kanamycin, tetracycline, streptomycin, or chloramphenicol. Examples of other suitable marker genes include antibiotic resistance genes such as those conferring resistance to G4 18 and hygromycin (npt-II, hyg-B); herbicide resistance genes such as those conferring resistance to phosphinothricin and sulfonamide based herbicides (bar and suI respectively; EP-A-242246, EP-A-0369637) and screenable markers such as beta-glucoronidase (GB2 197653), luciferase and green fluorescent protein. Suitable vectors for propagating the construct include, but are not limited to, plasmids, cosmids, bacteriophages or viruses.

The marker gene is preferably controlled by a second promoter which allows expression in cells other than the seed, thus allowing selection of cells or tissue containing the marker at any stage of development of the plant. Preferred second promoters are the promoter of nopaline synthase gene of Agrobacterium and the promoter derived from the gene which encodes the 35S subunit of cauliflower mosaic virus (CaMV) coat protein. However, any other suitable second promoter may be used.

The nucleic acid molecule encoding a plant glycogenin-like protein may be native or foreign to the plant into which it is introduced. One of the effects of introducing a nucleic acid molecule encoding a plant glycogenin-like gene into a plant is to increase the amount of plant glycogenin-like protein present and therefore the amount of starch produced by increasing the copy number of the nucleic acid molecule. Foreign plant glycogenin-like nucleic acid molecules may in addition have different temporal and/or spatial specificity for starch synthesis compared to the native plant glycogenin-like protein of the plant, and so may be useful in altering when and where or what type of starch is produced. Regulatory elements of the plant glycogenin-like genes may also be used in altering starch synthesis in a plant, for example by replacing the native regulatory elements in the plant or providing additional control mechanisms. The regulatory regions of the invention may confer expression of a plant glycogenin-like gene product in a chemically-inducible, dark-inducible, developmentally regulated, developmental-stage specific, wound-induced, environmental factor-regulated, organ-specific, cell-specific, tissue-specific, or constitutive manner. Alternatively, the expression conferred by a regulatory region may encompass more than one type of expression selected from the group consisting of chemically-inducible, dark-inducible, developmentally regulated, developmental-stage specific, wound-induced, environmental factor-regulated, organ-specific, cell-specific, tissue-specific, and constitutive.

Further, any of the nucleic acid molecules (including EST clone nucleic acid molecules) and/or polypeptides and proteins described herein, can be used as markers for qualitative trait loci in breeding programs for crop plants. To this end, the nucleic acid molecules, including, but not limited to, full length plant glycogenin-like genes coding sequences, and/or partial sequences (ESTs), can be used in hybridization and/or DNA amplification assays to identify the endogenous plant glycogenin-like genes, plant glycogenin-like gene mutant alleles and/or plant glycogenin-like gene expression products in cultivars as compared to wild-type plants. They can also be used as markers for linkage analysis of qualitative trait loci. It is also possible that the plant glycogenin-like genes may encode a product responsible for a qualitative trait that is desirable in a crop breeding program. Alternatively, the plant glycogenin-like protein and/or peptides can be used as diagnostic reagents in immunoassays to detect expression of the plant glycogenin-like genes in cultivars and wild-type plants.

Genetically-engineered plants containing constructs comprising the plant glycogenin-like nucleic acid and a reporter gene can be generated using the methods described herein for each plant glycogenin-like nucleic acid gene variant, to screen for loss-of-function variants induced by mutations, including but not limited to, deletions, point mutations, rearrangements, translocation, etc. The constructs can encode for fusion proteins comprising a plant glycogenin-like protein fused to a protein product encoded by a reporter gene. Alternatively, the constructs can encode for a plant glycogenin-like protein and a reporter gene product that are not fused. The constructs may be transformed into the homozygous recessive plant glycogenin-like gene mutant background, and the restorative phenotype examined, i.e. quantity and quality of starch, as a complementation test to confirm the functionality of the variants isolated.

### 5.2 Plant Glycogenin-Like Gene Products

The invention encompasses the polypeptides of SEQ ID NOs: 3, 7, 11, 13, 15, 17, 19, 21, 22, 24, 26, 28, 30, 31, 32, or 34. Plant glycogenin-like proteins, polypeptides and peptide fragments, variants, allelic variants, mutated, truncated or deleted forms of plant glycogenin-like proteins and/or plant glycogenin-like fusion proteins can be prepared for a variety of uses, including, but not limited to, the generation of antibodies, as reagents in assays, the identification of other cellular gene products involved in starch synthesis and/or starch synthesis initiation, etc.

Plant glycogenin-like translational products include, but are not limited to those proteins and polypeptides encoded by the sequences of the plant glycogenin-like nucleic acid molecules of the invention. The invention encompasses proteins that are functionally equivalent to the plant glycogenin-like gene products of the invention.

The primary use of the plant glycogenin-like gene products of the invention is to alter starch synthesis via increasing the number of priming or initiation sites for elongation of glucose chains.

In an embodiment of the invention, an isolated polypeptide comprises the amino acid molecule of SEQ ID NO: 9, or a variant or fragment thereof provided the amino acid sequence is not that of SEQ ID NO: 35.

The present invention also provides variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, deleting one or more of the receiver domains. Thus, specific biological effects can be elicited by addition of a variant of limited function.

Modification of the structure of the subject polypeptides can be for such purposes as enhancing efficacy, stability, or post-translational modifications (e.g., to alter the phosphorylation pattern of the protein). Such modified peptides, when designed to retain at least one activity of the naturally-occurring form of the protein, or to produce specific antagonists thereof, are considered functional equivalents of the polypeptides. Such modified peptides can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule.

Whether a change in the amino acid sequence of a peptide results in a functional homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

In a preferred embodiment, a mutant polypeptide that is a variant of a polypeptide of the invention can be assayed for: (1) the ability to complement glycogenin function in a yeast or plant system in which the native glycogenin or plant glygogenin-like genes have been knocked out; (2) the ability to form a complex with a glucose or oligosaccharide; or (3) the ability to promote initiation of elongation of polysaccharide chains.

The invention encompasses functionally equivalent mutant plant glycogenin-like proteins and polypeptides. The invention also encompasses mutant plant glycogenin-like proteins and polypeptides that are not functionally equivalent to the gene products. Such a mutant plant glycogenin-like protein or polypeptide may contain one or more deletions, additions or substitutions of plant glycogenin-like amino acid residues within the amino acid sequence encoded by any one the plant glycogenin-like nucleic acid molecules described above in Section 5.1, and which result in loss of one or more functions of the plant glycogenin-like protein, thus producing a plant glycogenin-like gene product not functionally equivalent to the wild-type plant glycogenin-like protein.

Plant glycogenin-like proteins and polypeptides bearing mutations can be made to plant glycogenin-like DNA (using techniques discussed above as well as those well known to one of skill in the art) and the resulting mutant plant glycogenin-like proteins tested for activity. Mutants can be isolated that display increased function, (e.g., resulting in improved root formation), or decreased function (e.g., resulting in suboptimal root function). In particular, mutated plant glycogenin-like proteins in which any of the exons shown in SEQ ID NO: 1 are deleted or mutated are within the scope of the invention. Additionally, peptides corresponding to one or more exons of the plant glycogenin-like protein, truncated or deleted plant glycogenin-like protein are also within the scope of the invention. Fusion proteins in which the full length plant glycogenin-like protein or a plant glycogenin-like polypeptide or peptide fused to an unrelated protein are also within the scope of the invention and can be designed on the basis of the plant glycogenin-like nucleotide and plant glycogenin-like amino acid sequences disclosed herein.

While the plant glycogenin-like polypeptides and peptides can be chemically synthesized (e.g., see Creighton, 1983, Proteins: Structures and Molecular Principles, W. H. Freeman & Co., NY) large polypeptides derived from plant glycogenin-like gene and the full length plant glycogenin-like gene may advantageously be produced by recombinant DNA technology using techniques well known to those skilled in the art for expressing nucleic acid molecules.

Nucleotides encoding fusion proteins may include, but are not limited to, nucleotides encoding full length plant glycogenin-like proteins, truncated plant glycogenin-like proteins, or peptide fragments of plant glycogenin-like proteins fused to an unrelated protein or peptide, such as for example, an enzyme, fluorescent protein, or luminescent protein that can be used as a marker or an epitope that facilitates affinity-based purificaiton. Alternatively, the fusion protein can further comprise a heterologous protein such as a transit peptide or fluorescence protein.

In an embodiment of the invention, the percent identity between two polypeptides of the invention is at least 40%. In a preferred embodiment of the invention, the percent identity between two polypeptides of the invention is at least 50%. In another embodiment, the percent the percent identity between two polypeptides of the invention is at least 60%, 70%, 80%, 90%, 95%, 96%, 97%, or at least 98%. Determining whether two sequences are substantially similar may be carried out using any methodologies known to one skilled in the art, preferably using computer assisted analysis as described in section 5.1.

Further, it may be desirable to include additional DNA sequences in the protein expression constructs. Examples of additional DNA sequences include, but are not limited to, those encoding: a 3′ untranslated region; a transcription termination and polyadenylation signal; an intron; a signal peptide (which facilitates the secretion of the protein); or a transit peptide (which targets the protein to a particular cellular compartment such as the nucleus, chloroplast, mitochondria or vacuole). The nucleic acid molecules of the invention will preferably comprise a nucleic acid molecule encoding a transit peptide, to ensure delivery of any expressed protein to the plastid. Preferably the transit peptide will be selective for plastids such as amyloplasts or chloroplasts, and can be native to the nucleic acid molecule of the invention or derived from known plastid sequences, such as those from the small subunit of the ribulose bisphosphate carboxylase enzyme (ssu of rubisco) from pea, maize or sunflower for example. Transit peptide comprising amino acid residues 1-195 of SEQ ID NO: 2 is an example of a transit peptide native to the polypeptide of the invention. Where an agonist or antagonist which modulates activity of the plant glycogenin-like protein is a polypeptide, the polypeptide itself must be appropriately targeted to the plastids, for example by the presence of plastid targeting signal at the N terminal end of the protein (Castro Silva Filho et al Plant Mol Biol 30 769-780 (1996) or by protein-protein interaction (Schenke PC et al, Plant Physiol 122 235-241 (2000) and Schenke et al PNAS 98(2) 765-770 (2001). The transit peptides of the invention are used to target transportation of plant glycogenin-like proteins as well as agonists or antagonists thereof to plastids, the sites of starch synthesis, thus altering the starch synthesis process and resulting starch characteristics.

The plant glycogenin-like proteins and transit peptides associated with the plant glycogenin-like genes of the present invention have a number of important agricultural uses. The transit peptides associated with the plant glycogenin-like genes of the invention may be used, for example, in transportation of desired heterologous gene products to a root, a root modified through evolution, tuber, stem, a stem modified through evolution, seed, and/or endosperm of transgenic plants transformed with such constructs.

The invention encompasses methods of screening for agents (i.e., proteins, small molecules, peptides) capable of altering the activity of a plant glycogenin-like protein in a plant. Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into nucleic acid molecules such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. In one embodiment, the antigenic peptide of a protein of the invention or fragments or immunogenic fragments of a protein of the invention comprise at least 8 (preferably 10, 15, 20, 30 or 35) consecutive amino acid residues of the amino acid sequence of SEQ ID NO: 3, 7, 9, 11, 13, 15, 17, 19, 21, 22, 24, 26, 28, 30, 32, or 34 and encompasses an epitope of the protein such that an antibody raised against the peptide forms a specific immune complex with the protein.

Exemplary amino acid sequences of the polypeptides of the invention can be used to generate antibodies against plant glycogenin-like genes. In one embodiment, the immunogenic polypeptide is conjugated to keyhole limpet hemocyanin (“KLH”) and injected into rabbits. Rabbit IgG polyclonal antibodies can purified, for example, on a peptide affinity column. The antibodies can them be used to bind to and identify the polypeptides of the invention that have been extracted and separated via gel electrophoresis or other means.

One aspect of the invention pertains to isolated plant glycogenin-like polypeptides of the invention, variants thereof, as well as variants suitable for use as immunogens to raise antibodies directed against a plant glycogenin-like polypeptide of the invention. In one embodiment, the native polypeptide can be isolated, using standard protein purification techniques, from cells or tissues expressing a plant glycogenin-like polypeptide. In a preferred embodiment, plant glycogenin-like polypeptides of the invention are produced from expression vectors by recombinant DNA techniques. In another preferred embodiment, a polypeptide of the invention is synthesized chemically using standard peptide synthesis techniques.

An isolated or purified protein or biologically active portion thereof is substantially free of cellular material or other contaminating proteins from the cell or tissue source from which the protein is derived, or substantially free of chemical precursors or other chemicals when chemically synthesized. The language “substantially free” indicates protein preparations in which the protein is separated from cellular components of the cells from which it is isolated or recombinantly produced. Thus, protein that is substantially free of cellular material includes protein preparations having less than 20%, 10%, or 5% (by dry weight) of a contaminating protein. Similarly, when an isolated plant glycogenin-like polypeptide of the invention is recombinantly produced, it is substantially free of culture medium. When the plant glycogenin-like polypeptide is produced by chemical synthesis, it is preferably substantially free of chemical precursors or other chemicals.

Biologically active portions of a polypeptide of the invention include polypeptides comprising amino acid sequences identical to or derived from the amino acid sequence of the protein, such that the variants sequences comprise conservative substitutions or truncations (e.g., amino acid sequences comprising fewer amino acids than those shown in any of SEQ ID NOs: 3, 7, 9, 11, 13, 15, 17, 19, 21, 22, 24, 26, 28, 30, 32, and 34, but which maintain a high degree of homology to the remaining amino acid sequence). Typically, biologically active portions comprise a domain or motif with at least one activity of the corresponding protein. Domains or motifs include, but are not limited to, a biologically active portion of a protein of the invention can be a polypeptide which is, for example, at least 10, 25, 50, 100, 200, 300, 400 or 500 amino acids in length. Polypeptides of the invention can comprise, for example, a glycosylation domain or site for complexing with polysaccharide or for attachment of disaccharide or a monomeric unit thereof, or a site that interacts with starch synthase and other enzymes that act on the polysaccharide.

### 5.3 Production of Transgenic Plants and Plant Cells

The invention also encompasses transgenic or genetically-engineered plants, and progeny thereof. As used herein, a transgenic or genetically-engineered plant referes to a plant and a portion of its progeny which comprises a nucleic acid molecule which is not native to the initial parent plant. The introduced nucleic acid molecule may originate from the same species e.g., if the desired result is over-expression of the endogenous gene, or from a different species. A transgenic or genetically-engineered plant may be easily identified by a person skilled in the art by comparing the genetic material from a non-transformed plant, and a plant produced by a method of the present invention for example, a transgenic plant may comprise multiple copies of plant glycogenin-like genes, and/or foreign nucleic acid molecules. Transgenic plants are readily distinguishable from non-transgenic plants by standard techniques. For example a PCR test may be used to demonstrate the presence or absence of introduced genetic material. Transgenic plants may also be distinguished from non-transgenic plants at the DNA level by Southern blot or at the RNA level by Northern blot or at the protein level by western blot, by measurement of enzyme activity or by starch composition or properties.

The nucleic acids of the invention may be introduced into a cell by any suitable means. Preferred means include use of a disarmed Ti-plasmid vector carried by Agrobacterium by procedures known in the art, for example as described in EP-A-0116718 and EP-A-0270822. Agrobacterium mediated transformation methods are now available for monocots, for example as described in EP 0672752 and WO00/63398. Alternatively, the nucleic acid may be introduced directly into plant cells using a particle gun. A further method would be to transform a plant protoplast, which involves first removing the cell wall and introducing the nucleic acid molecule and then reforming the cell wall. The transformed cell can then be grown into a plant.

In an embodiment of the present invention, Agrobacterium is employed to introduce the gene constructs into plants. Such transformations preferably use binary Agrobacterium T-DNA vectors (Bevan, 1984, Nuc. Acid Res. 12:8711-21), and the co-cultivation procedure (Horsch et al., 1985, Science 227:1229-31). Generally, the Agrobacterium transformation system is used to engineer dicotyledonous plants (Bevan et al., 1982, Ann. Rev. Genet. 16:357-84; Rogers et al., 1986, Methods Enzymol. 118:627-41). The Agrobacterium transformation system may also be used to transform, as well as transfer, DNA to monocotyledonous plants and plant cells (see Hernalsteen et al., 1984, EMBO J. 3:3039-41; Hooykass-Van Slogteren et al., 1984, Nature 311:763-4; Grimsley et al., 1987, Nature 325:1677-79; Boulton et al., 1989, Plant Mol. Biol. 12:31-40.; Gould et al., 1991, Plant Physiol. 95:426-34).

Various alternative methods for introducing recombinant nucleic acid constructs into plants and plant cells may also be utilized. These other methods are particularly useful where the target is a monocotyledonous plant or plant cell. Alternative gene transfer and transformation methods include, but are not limited to, protoplast transformation through calcium-, polyethylene glycol (PEG)- or electroporation-mediated uptake of naked DNA (see Paszkowski et al., 1984, EMBO J. 3:2717-22; Potrykus et al., 1985, Mol. Gen. Genet. 199:169-177; Fromm et al., 1985, Proc. Natl. Acad. Sci. USA 82:5824-8; Shimamoto, 1989, Nature 338:274-6), and electroporation of plant tissues (D'Halluin et al., 1992, Plant Cell 4:1495-1505). Additional methods for plant cell transformation include microinjection, silicon carbide mediated DNA uptake (Kaeppler et al., 1990, Plant Cell Reporter 9:415-8), and microprojectile bombardment (Klein et al., 1988, Proc. Natl. Acad. Sci. USA 85:4305-9; Gordon-Kamm et al., 1990, Plant Cell 2:603-18).

According to the present invention, desired plants and plant cells may be obtained by engineering the gene constructs described herein into a variety of plant cell types, including, but not limited to, protoplasts, tissue culture cells, tissue and organ explants, pollen, embryos as well as whole plants. In an embodiment of the present invention, the engineered plant material is selected or screened for transformants (i.e., those that have incorporated or integrated the introduced gene construct or constructs) following the approaches and methods described below. An isolated transformant may then be regenerated into a plant. Alternatively, the engineered plant material may be regenerated into a plant, or plantlet, before subjecting the derived plant, or plantlet, to selection or screening for the marker gene traits. Procedures for regenerating plants from plant cells, tissues or organs, either before or after selecting or screening for marker gene or genes, are well known to those skilled in the art.

A transformed plant cell, callus, tissue or plant may be identified and isolated by selecting or screening the engineered plant material for traits encoded by the marker genes present on the transforming DNA. For instance, selection may be performed by growing the engineered plant material on media containing inhibitory amounts of the antibiotic or herbicide to which the transforming marker gene construct confers resistance. Further, transformed plants and plant cells may also be identified by screening for the activities of any visible marker genes (e.g., the β-glucuronidase, luciferase, green fluorescent protein, B or C1 anythocyanin genes) that may be present on the recombinant nucleic acid constructs of the present invention. Such selection and screening methodologies are well known to those skilled in the art.

The present invention is applicable to all plants which produce or store starch. Examples of such plants are cereals such as maize, wheat, rice, sorghum, barley; fruit producing species such as banana, apple, tomato or pear; root crops such as cassaya, potato, yam, beet or turnip; oilseed crops such as rapeseed, canola, sunflower, oil palm, coconut, linseed or groundnut; meal crops such as soya, bean or pea; and any other suitable species.

In a preferred embodiment of the present invention, the method comprises the additional step of growing the plant and harvesting the starch from a plant part. In order to harvest the starch, it is preferred that the plant is grown until plant parts containing starch develop, which may then be removed. In a further preferred embodiment, the propagating material from the plant may be removed, for example the seeds. The plant part can be an organ such as a stem, root, leaf, or reproductive body. Alternatively, the plant part may be a modified organ such as a tuber, or the plant part is a tissue such as endosperm.

### 5.4 Transgenic Plants That Ectopically Express Plant Glycogenin-Like Protein

According to one aspect of the invention, the nucleic acid molecule expressed in the plant cell, plant, or part of a plant that comprises a nucleotide sequence encoding a plant glycogenin-like protein, fragment of variant thereof. The nucleic acid molecule expressed in the plant cell can comprise a nucleotide sequence encoding a full length plant glycogenin-like protein. Examples of such sequences include SEQ ID NOs: 1, 2, 6, 8, 10, 12, and 14, or variants thereof and the corresponding the amino acid sequences of SEQ ID NOs: 3, 7, 9, 11, 13, and 15 or variants thereof.

In an embodiment of the invention, the nucleic acid molecules of the invention are expressed in a plant cell and are transcribed only in the sense orientation. A plant that expresses a recombinant plant glycogenin-like nucleic acid may be engineered by transforming a plant cell with a nucleic acid construct comprising a regulatory region operably associated with a nucleic acid molecule, the sequence of which encodes a plant glycogenin-like protein or a fragment thereof. In plants derived from such cells, starch synthesis is altered in ways described in section 5.6. The term “operably associated” is used herein to mean that transcription controlled by the associated regulatory region would produce a functional mRNA, whose translation would produce the plant glycogenin-like protein. Starch may be altered in particular parts of a plant, including but not limited to seeds, tubers, leaves, roots and stems or modifications thereof.

In an embodiment of the invention, a plant is engineered to constitutively express a plant glycogenin-like protein in order to alter the starch content of the plant. In a preferred embodiment, the starch content is 40%, 30%, 20%, 10%, 5%, 2% greater than that of anon-engineered control plant(s). In another preferred embodiment, the starch content is 40%, 30%, 20%, 10%, 5%, 2% less than that of a non-engineered control plant(s).

In another aspect of the invention, where the nucleic acid molecules of the invention are expressed in a plant cell and are transcribed only in the sense orientation, the starch content of the plant cell and plants derived from such a cells exhibit altered starch content. The altered starch content comprises an increase in the ratio of amylose to amylopectin. In one embodiment of the invention, the ratio of amylose to amylopectin increases by 2%, 5%, 10%, 20%, 30%, 40%, or 50% in comparison to a non-engineered control plant(s).

In preferred embodiment of the invention, the nucleic acid molecules of the invention are expressed in a potato plant and are transcribed only in the sense orientation. The starch content of the plant, including the tubers, exhibit increased starch content. If the number of copies of the nucleic acid molecules of the invention are expressed in a potato plant that are transcribed only in the sense orientation is increased, the starch content of the plant, including the tubers, increases.

In yet another embodiment of the present invention, it may be advantageous to transform a plant with a nucleic acid construct operably linking a modified or artificial promoter to a nucleic acid molecule having a sequence encoding a plant glycogenin-like protein or a fragment thereof. Such promoters typically have unique expression patterns and/or expression levels not found in natural promoters because they are constructed by recombining structural elements from different promoters. See, e.g., Salina et al., 1992, Plant Cell 4:1485-93, for examples of artificial promoters constructed from combining cis-regulatory elements with a promoter core.

In a preferred embodiment of the present invention, the associated promoter is a strong root and/or embryo-specific plant promoter such that the plant glycogenin-like protein is overexpressed in the transgenic plant.

In yet another preferred embodiment of the present invention, the overexpression of plant glycogenin-like protein in starch producing organs and organelles may be engineered by increasing the copy number of the plant glycogenin-like gene. One approach to producing such transgenic plants is to transform with nucleic acid constructs that contain multiple copies of the complete plant glycogenin-like gene with native or heterolgous promoters. Another approach is repeatedly transform successive generations of a plant line with one or more copies of the complete plant glycogenin-like gene constructs. Yet another approach is to place a complete plant glycogenin-like gene in a nucleic acid construct containing an amplification-selectable marker (ASM) gene such as the glutamine synthetase or dihydrofolate reductase gene. Cells transformed with such constructs is subjected to culturing regimes that select cell lines with increased copies of complete plant glycogenin-like gene. See, e.g., Donn et al., 1984, J. Mol. Appl. Genet. 2:549-62, for a selection protocol used to isolate of a plant cell line containing amplified copies of the GS gene. Cell lines with amplified copies of the plant glycogenin-like gene can then be regenerated into transgenic plants.

### 5.5 Transgenic Plants That Suppress Endogenous Plant Glycogenin-Like Protein Expression

The nucleic acid molecules of the invention may also be used to augment the starch riming activity of a plant cell, plant, or part of a plant, or alternatively to alter activity of the plant glycogenin-like protein of a plant cell, plant, or part of a plant by modifying transcription or translation of the plant glycogenin-like gene. In an embodiment of the invention, an antagonist which is capable of altering the expression of a nucleic acid molecule of the invention is introduced into a plant in order to alter the synthesis of starch. The antagonist may be protein, nucleic acid, chemical antagonist, or any other suitable moiety. In an embodiment of the invention, an antagonist which is capable of altering the expression of a nucleic acid molecule of the invention is provided to alter the synthesis of starch. The antagonist may be protein, nucleic acid, chemical antagonist, or any other suitable moiety. Typically, the antagonist will function by inhibiting or enhancing transcription from the plant glycogenin-like gene, either by affecting regulation of the promoter or the transcription process; inhibiting or enhancing translation of any RNA product of the plant glycogenin-like gene; inhibiting or enhancing the activity of the plant glycogenin-like protein itself or inhibiting or enhancing the protein-protein interaction of the plant glycogenin-like protein and downstream enzymes of the starch biosynthesis pathway. For example, where the antagonist is a protein it may interfere with transcription factor binding to the plant glycogenin-like gene promoter, mimic the activity of a transcription factor, compete with or mimic the plant glycogenin-like protein, or interfere with translation of the plant glycogenin-like RNA, interfere with the interaction of the plant glycogenin-like protein and downstream enzymes. Antagonists which are nucleic acids may encode proteins described above, or may be transposons which interfere with expression of the plant glycogenin-like gene.

The suppression may be engineered by transforming a plant with a nucleic acid construct encoding an antisense RNA or ribozyme complementary to a segment or the whole of plant glycogenin-like gene RNA transcript, including the mature target mRNA. In another embodiment, plant glycogenin-like gene suppression may be engineered by transforming a plant cell with a nucleic acid construct encoding a ribozyme that cleaves the plant glycogenin-like gene mRNA transcript.

In another embodiment, the plant glycogenin-like mRNA transcript can be suppressed through the use of RNA interference, referred to herein as RNAi. RNAi allows for selective knock out of a target gene in a highly effective and specific manner. The RNAi technique involves introducing into a cell double-stranded RNA (dsRNA) which corresponds to exon portions of a target gene such as an endogenous plant glycogenin-like gene. The dsRNA causes the rapid destruction of the target gene's messenger RNA, i.e. an endogenous plant glycogenin-like gene mRNA, thus preventing the production of the plant glycogenin-like protein encoded by that gene. The RNAi constructs of the invention confer expression of dsRNA which correspond to exon portions of an endogenous plant glycogenin-like gene. The strands of RNA that form the dsRNA are complimentary strands from encoded by coding region, i.e., exons encoding sequence, on the 3′ end of the plant glycogenin-like gene.

The dsRNA has an effect on the stability of the mRNA. The mechanism of how dsRNA results in the loss of the targeted homologous mRNA is still not well understood (Cogoni and Macino, 2000, Genes Dev 10: 638-643; Guru, 2000, Nature 404, 804-808; Hammond et al., 2001, Nature Rev Gen 2: 110-119). Current theories suggest a catalytic or amplification process occurs that involves initiation step and an effector step.

In the initiation step, input dsRNA is digested into 21-23 nucleotide “guide RNAs”. These guide RNAs are also referred to as siRNAs, or short interfering RNAs. Evidence indicates that siRNAs are produced when a nuclease complex, which recognizes the 3′ ends of dsRNA, cleaves dsRNA (introduced directly or via a transgene or virus) ˜22 nucleotides from the 3′ end. Successive cleavage events, either by one complex or several complexes, degrade the RNA to 19-20 bp duplexes (siRNAs), each with 2-nucleotide 3′ overhangs. RNase III-type endonucleases cleave dsRNA to produce dsRNA fragments with 2-nucleotide 3′ tails, thus an RNase III-like activity appears to be involved in the RNAi mechanism. Because of the potency of RNAi in some organisms, it has been proposed that siRNAs are replicated by an RNA-dependent RNA polymerase (Hammond et al., 2001, Nature Rev Gen 2:110-119; Sharp, 2001, Genes Dev 15: 485-490).

In the effector step, the siRNA duplexes bind to a nuclease complex to form what is known as the RNA-induced silencing complex, or RISC. The nuclease complex responsible for digestion of mRNA may be identical to the nuclease activity that processes input dsRNA to siRNAs, although its identity is currently unclear. In either case, the RISC targets the homologous transcript by base pairing interactions between one of the siRNA strands and the endogenous mRNA. It then cleaves the mRNA ˜12 nucleotides from the 3′ terminus of the siRNA (Hammond et al., 2001, Nature Rev Gen 2:110-119; Sharp, 2001, Genes Dev 15: 485-490).

Methods and procedures for successful use of RNAi technology in post-transcriptional gene silencing in plant systems has been described by Waterhouse et al. (Waterhouse et al., 1998, Proc Natl Acad Sci USA, 95(23):13959-64). Methods specific to construction of the RNAi constructs of the invention can be found in Examples 2 and 6 as well as FIGS. 6 and 10. While the invention encompasses use of any plant glycogenin-like gene of the invention in the RNAi constructs, in a preferred embodiment, the strands of RNA that form the dsRNA are complimentary strands encoded by a coding region on the 3′ end from nucleotide residues 1196-1662 of SEQ ID NO:2.

For all of the aforementioned suppression or antisense constructs, it is preferred that such nucleic acid constructs express specifically in organs where starch synthesis occurs (i.e. tubers, seeds, stems roots and leaves) and/or the plastids where starch synthesis occurs. Alternatively, it may be preferred to have the suppression or antisense constructs expressed constitutively. Thus, constitutive promoters, such as the nopaline, CaMV 35S promoter, may also be used to express the suppression constructs. A most preferred promoter for these suppression or antisense constructs is a rice actin promoter. Alternatively, a co-suppression construct promoter can be one that expresses with the same tissue and developmental specificity as the plant glycogenin-like gene.

In accordance with the present invention, desired plants with suppressed target gene expression may also be engineered by transforming a plant cell with a co-suppression construct. A co-suppression construct comprises a functional promoter operatively associated with a complete or partial plant glycogenin-like nucleic acid molecule. According to the present invention, it is preferred that the co-suppression construct encodes fully functional plant glycogenin-like gene mRNA or enzyme, although a construct encoding a an incomplete plant glycogenin-like gene mRNA may also be useful in effecting co-suppression.

In accordance with the present invention, desired plants with suppressed target gene expression may also be engineered by transforming a plant cell with a construct that can effect site-directed mutagenesis of the plant glycogenin-like gene. For discussions of nucleic acid constructs for effecting site-directed mutagenesis of target genes in plants see, e.g., Mengiste et al., 1999, Biol. Chem. 380:749-758; Offringa et al., 1990, EMBO J. 9:3077-84; and Kanevskii et al., 1990, Dokl. Akad. Nauk. SSSR 312:1505-7. It is preferred that such constructs effect suppression of plant glycogenin-like genes by replacing the endogenous plant glycogenin-like gene nucleic acid molecule through homologous recombination with either an inactive or deleted plant glycogenin-like protein coding nucleic acid molecule.

In yet another embodiment, antisense technology can be used to inhibit plant glycogenin-like gene mRNA expression. Alternatively, the plant can be engineered, e.g., via targeted homologous recombination to inactive or “knock-out” expression of the plant's endogenous plant glycogenin-like protein. The plant can be engineered to express an antagonist that hybridizes to one or more regulatory elements of the gene to interfere with control of the gene, such as binding of transcription factors, or disrupting protein-protein interaction. The plant can also be engineered to express a co-suppression construct. The suppression technology may also be useful in down-regulating the native plant glycogenin-like gene of a plant where a foreign plant glycogenin-like gene has been introduced. To be effective in altering the activity of a plant glycogenin-like protein in a plant, it is preferred that the nucleic acid molecules are at least 50, preferably at least 100 and more preferably at least 150 nucleotides in length. In one aspect of the invention, the nucleic acid molecule expressed in the plant cell can comprise a nucleotide sequence of the invention which encodes a full length plant glycogenin-like protein and wherein the nucleic acid molecule has been transcribed only in the antisense direction.

In a particular embodiment of the invention, a plant is engineered to express a dsRNA homologous to a portion of the coding region of an endogeneous PGSIP or a plant glycogenin-like gene transcribed in the antisense direction in order to alter the starch content of the plant. In a preferred embodiment, the starch content is 40%, 30%, 20%, 10%, 5% less than that of a non-engineered control plant(s). In a another preferred embodiment, starch is absent from certain plant organs or tissues in comparison to a non-engineered control plant(s). In one embodiment starch content is decreased or absent in the leaves of plants engineered using the antisense technology described herein when compared to the starch content in a non-engineered control plant(s). In other embodiments the starch content of tubers, or seeds is decreased or absent in plants engineered using the antisense technology described herein when compared to the starch content in a non-engineered control plant(s). Plant tissues in which starch content can be decreased using the methods of the invention include but are not limited to endospern, leaf mesophyll, and root or stem cortex or pith.

In another aspect of the invention, the nucleic acid molecules of the invention are expressed in a plant cell engineered expressing a dsRNA homologous to a portion of the coding region of an endogeneous PGSIP or using the antisense technology described herein and the starch content of the plant cell and plants derived from such a cells exhibit altered starch content. The altered starch content comprises an decrease in the ratio of amylose to amylopectin. In one embodiment of the invention, the ratio of amylose to amylopectin decreases by 10%, 20%, 30%, 40%, or 50% in comparison to a non-engineered control plant(s).

In a particular embodiment, the nucleic acid molecules of the invention are expressing a dsRNA homologous to a portion of the coding region of an endogeneous PGSIP or using the antisense technology described herein, in conjunction with a developmental specific promoter directed towards later stages of development. In this particular embodiment, starch content in leaves of a plant can decrease, while starch content in other organs and tissues of a plant are altered in the same or different ways.

In another particular embodiment, the nucleic acid molecules of the invention are expressing a dsRNA homologous to a portion of the coding region of an endogeneous PGSIP or using the antisense technology described herein in conjunction with a developmental specific promoter directed towards later stages of seed development, in cereals crops. In this embodiment, the ratio of small starch granules to large starch granules increases. An increased ratio of small to large starch granules results in greater accessibility of starch granules, which has certain industrial and commercial advantages related to extraction and processing of starch.

The progeny of the transgenic or genetically-engineered plants of the invention containing the nucleic acids of the invention are also encompassed by the invention.

### 5.6 Modified Starch

The invention encompasses methods of altering starch synthesis in a plant and the resulting modified starch produced.

In the context of the present invention, “altering starch synthesis” means altering any aspect of starch production in the plant, from initiation by the starch primer to downstream aspects of starch production such as elongation, branching and storage, such that it differs from starch synthesis in the native plant. In the invention, this is achieved by altering the activity of the starch primer, which includes, but is not limited to, its function in initiating starch synthesis, its temporal and spatial distribution and specificity, and its interaction with downstream factors in the synthesis pathway. The effects of altering the activity of the starch primer may include, for example, increasing or decreasing the starch yield of the plant; increasing or decreasing the rate of starch production; altering temporal or spatial aspects of starch production in the plant; altering the initiation sites of starch synthesis; changing the optimum conditions for starch production; and altering the type of starch produced, for example in terms of the ratio of its different components. For example, the endosperm of mature wheat and barley grains contain two major classes of starch granules: large, early formed “A” granules and small, later formed “B” granules. Type A starch granules in wheat are about 20 μm diameter and type B around 5 μm in diameter (Tester, 1997, in: Starch Structure and Functionality, Frazier et al., eds., Royal Society of Chemistry, Cambridge, UK). Rice starch granules are typically less than 5 μm in diameter, while potato starch granules can be greater than 80 μm in diameter. The quality of starch in wheat and barley is greatly influenced by the ratio of A-granules to B-granules. Altering the activity of the starch primer will influence the number of granule initiation sites, which will be an important factor in determining the number and size of formed starch granules. The degree to which the starch priming activity of the plant is affected will depend at least upon the nature and of the nucleic acid molecule or antagonist introduced into the plant, and the amount present. By altering these variables, a person skilled in the art can regulate the degree to which starch synthesis is altered according to the desired end result.

The methods of the invention (i.e. engineering-a plant to express a construct comprising a plant glycogenin-like nucleic acid) can, in addition to altering the total quantity of starch, alter the fine structure of starch in several ways including but not limited to, altering the ratio of amylose to amylopectin, altering the length of amylose chains, altering the length of chains of amylopectin fractions of low molecular weight or high molecular weight fractions, or altering the ratio of low molecular weight or high molecular weight chains of amylopectin. The methods of the invention can also be utilized to alter the granule structure of starch, i.e. the ratio of large to small starch granules from a plant or a portion of a plant. The alteration in the structure of starch can in turn affect the functional characteristics of starch such as viscosity, elasticity, or Theological properties of the starch such as those measured using viscometric analysis. The modified starch can also be characterized by an alteration of more than one of the above-mentioned properties.

In an embodiment the length of amylose chains in starch extracted from a plant engineered express a construct comprising a plant glycogenin-like nucleic acid is decreased by at least 50, 100, 150, 200, 250, or 300 glucose units in length in comparison to amylose from non-modified starch from a plant of the same genetic background. In another embodiment, the length of amylose chains in starch is increased by at least 50, 100, 150, 200, 250, or 300 glucose units in length in comparison to amylose from non-modified starch from a plant of the same genetic background.

In an embodiment of the invention, the ratio of amylose to amylopectin decreases by 10%, 20%, 30%, 40%, or 50% in comparison to a non-engineered control plant(s).

In a preferred embodiment, the ratio of low molecular weight chains to high molecular weight chains of amylopectin is altered by 10%, 20%, 30%, 40%, or 50% in comparison to a non-engineered control plant(s).

In another preferred embodiment the average length of low molecular weight chains 35 of amylopectin is altered by 5, 10, 15, 20, or 25 glucose units in length in comparison to a non-engineered control plant(s). In yet another preferred embodiment the average length of high molecular weight chains of amylopectin is altered by 10, 20, 30, 40, 50, 60, 70, or 80 glucose units in length in comparison to a non-engineered control plant(s).

According to one aspect of the invention, the ratio of small starch granules to large granules is altered by at least 10%, 20%, 30%, 40%, 50%, 60%, 70%, 80%, 90% or more in comparison to a non-engineered control plant(s).

In another aspect, the invention provides a complex comprising plant glycogenin-like proteins and plant polysaccharides. The inventors believe that members of the family of plant glycogenin-like proteins serve as primers for biosynthesis of a range of polysaccharides in plants, including but not limited to starch, hemicelluloses, and cellulose. The plant polysaccharides may be either homopolysaccharides comprising only a single type of monomeric unit or a heteropolysaccharides comprising two or more different kinds of monomeric units. Accordingly, it is contemplated that plant glycogenin-like proteins form complexes with such polysaccharides and its subunits. Glycosylated plant glycogenin-like proteins are encompassed in the invention. In the broadest sense, the invention encompasses a complex comprising a plant glycogenin-like protein and a number of monomeric units also referred to as subunits of the polysaccharides. Examples of monomeric units include but are not limited to glucose, xylose, mannose, galactose, ribose, and rhamnose, and may be a hexose, or a pentose, wherein the number ranges from a single to thousands of monomeric units, and wherein the linkages between the subunits may vary resulting in linear and/or branched structures. For example, starch and precursors of starch comprise of glucose subunits joined by either alpha 1,4-glycosidic bonds or alpha 1,6-glycosidic linkages; cellulose and precursors of cellulose comprise glucose subunits joined by beta 1,4-glycosidic bonds. The number of monomeric units ranges from 1-3,2-5,4-10, 8-16, 15-30, 20-40, 30-60, 50-100, 75-200, 100-500, or 300-800 monomeric units. Alternatively, the number of monomeric units ranges from 1000-5000, 5000-10,000, or 10,000-15,000 monomeric units. Preferably, the polysaccharide or its precursor is attached to a hydroxyl group of a tyrosine residue of the plant glycogenin-like protein. Without being bound by any theory or any mechanism, during biosynthesis, additional subunits, either singly or as oligosaccharides are added to the complex such that the total number of subunits increase over a period of time.

In one embodiment, the invention encompasses complexes comprising plant glycogenin-like protein and starch. In a specific embodiment, the complexes of plant glycogenin-like protein and starch are purified. The starch molecule or its precursor including a single glucose subunit, can be attached to a hydroxyl group of a tyrosine residue of the plant glycogenin-like protein. In various embodiments, in a population of complexes, the starch molecules that are complexed with the plant glycogenin-like proteins have different chain lengths and branching structures, for example, 1-3, 2-5, 4-10, 8-16, 15-30, 20-40, 30-60, 50-100, 75-200, 100-500, 200-700 glucose subunits. The polysaccharide complexed with the plant glycogenin-like proteins may consists of 2, 3, 4, 5, 6, 7, 8, 9, 10, 20, 30, 40, 50, 60, 70, 80, 90, 100, 110, 120, 130, 140, 150, 160, 170, 180, or 190 glucose subunits in length. In preferred embodiments of the invention, the polysaccharide is amylopectin, amylose, or a combination of both.

The complexes of the invention can be used to identify sites of starch synthesis in stages of plant development. Briefly, the glycogenin-like protein can be labeled by means described herein and the complexes from tissues, cells, or organs can then be separated by size and compared among different stages of development.

The embodiments described in each section above apply to the other aspects of the invention, mutatis mutandis.

### 6 EXAMPLES

### Example 1

### Identification of Plant Glycogenin-Like Gene Homologues in Arabidopsis

Arabidopsis nucleic acid molecules showing similarities to yeast glycogenin genes were identified by sequence analysis. The sequence analysis programs used in the following examples are from the Wisconsin Package of computer programs (Deveraux et al., Nucl. Acids Res. 12: 387 (1984); available from Genetics Computer Group, Madison, Wis.). ESTs and genes were identified using the program BLAST (Basic Local Alignment Search Tool; Altschul, S. F. et al (1990) J. Mol. Biol. 215:403-410, see also www.ncbi.nlm.nih.gov/BLAST/).

The sequence comparison and identification program tblastx was used with the yeast glycogenin 1 (Glg1) gene (GenBank:U25546, Swiss_Prot (SP):P36143) to search against the Arabidopsis sequences collected in an in-house database comprising published plant sequences. A number of hits to this gene were obtained. One of the hits was identified as EMBL:AC004260 version GI:2957150 which was annotated as “Sequencing in progress.” Therefore, the region showing homology to the yeast Glg1 gene was extracted and a protein sequence was predicted using GENSCAN (a protein prediction program, Burge, C. and Karlin, S. (1997), J.Mol.Biol., http://genes.mit.edu/GENSCANinfo.html). A blastp analysis using this protein showed strong homology to the glycogenin genes from C. elegans (8e-22), human (2e-19) and yeast (8e-06). A search in the database at NCBI at a later date showed that this gene is listed as T14N5.1 with the accession number EMBL:AC004260 (SPTREMBL:O80649) and annotated as “Unknown protein”. The protein sequence is set forth in SEQ ID NO: 6.

The in-house database described above was also searched with the yeast Glg2 gene (GB:U25436, SP:P47011) and the sequence identified above (accession EMBL:AC004260) using the program tblastn and tblastx. A number of further hits were identified. Out of the list of best hits, accession no. EMBL:AB026654, gene_id:MVE11.2 (SPTREMBL:Q9LSB1), showed strong homology to the glycogenin genes from C.elegans (le-21), GYG2 human (3e-21) and yeast (5e-06). The genomic sequence representing this gene was extracted and is shown in SEQ ID NO: 1. Further analysis by the organelle prediction programs PREDOTAR and/or TargetP (Emanuelsson et al., J. Mol. Biol. 300: 1005-1016 (2000)) showed that the protein comprises a transit peptide as shown in Table 1 below.

TABLE 1
TargetP V1.0 Prediction Results.
Number of input sequences: 1
Cleavage site predictions included.
Using PLANT networks.

| Name | Length | cTP | mTP | SP | Other | Loc. | RC | TPlen |
|---|---|---|---|---|---|---|---|---|
| AT3g18660 | 659 | 0.792 | 0.181 | 0.004 | 0.172 | C | 2 | 63 |
| cDNA | | | | | | | | |

Performing blastp analysis using this protein against yeast sequences in an in-house database clearly showed sequence similarities to the yeast Glg1 and Glg2 gene. were and a CD-ROM containing the full genome sequence of Arabidopsis was made available. A search of the Arabidopsis genome sequencing project database published (Nature 408: 791, (2000)) showed that EMBL:AB026654 corresponded to the sequence having accession no. AT3g18660. However AT3g18660 is reported to encode a protein of 575 amino acids whereas our analysis shows that this gene actually encodes a protein of 659 amino acids. A blastp analysis against the in-house database showed strong hits to five genes, EMBL:AC004260, AC000106, AC069144, AL035678 and AL035678 (corresponding to MIPS:at1g77130, at1g08990, at1g54940, at4 g33330 and at4 g33340). The sequences of these five genes are shown in SEQ ID NOs: 6, 8, 10, 12 and 14. The different accession numbers of these genes and their description in various databases are presented in Table 2.

TABLE 2
Accession numbers of the genes in various databases:

| MIPS | SPTREMBL | EMBL | GENE | Size | |
|---|---|---|---|---|---|
| AT3g18660 | Q9LSB1 | AB026654 | MVE11.2 | 659^{a} | aa |
| at1g77130 | O80649 | AC004260 | T14N5.1 | 1201 | aa |
| at1g08990 | O04031 | AC000106 | F7g19.14 | 546^{b} | aa |
| at1g54940 | Q9FZ37 | AC069144 | F14C21.47 | 557 | aa |
| at4g33330 | Q9SZB0 | AL035678 | F17M5.90 | 333 | aa |
| at4g33340 | Q9SZB1 | AL035678 | F17M5.100 | 277 | aa |

Note:
^{a}The AT3g18660 gene sequence in the MATDB (MIPS) database is reported to encode a 575 aa protein. The analysis performed by the inventors indicates that (exon 2) of the AT3g18660 gene is missing in the MATDB (MIPS) database sequence and present in sequences of the AT3g18660 gene found in other databases.
^{b}The at1g08990 gene accession in the MATDB (MIPS) database is reported to encode a protein of 550 aa in MATDB (MIPS). The at1g08990 gene accession in other databases is 546 aa in length.

TABLE 3
Comparison of AT3g18660 with other glycogenin-like genes from
Arabidopsis:

| | % identity nucleotide | % identity protein |
|---|---|---|
| AT3g18660 X at1g77130 | 68 | 65 |
| AT3g18660 X at1g08990 | 61 | 50 |
| AT3g18660 X at1g54940 | 61 | 49 |
| AT3g18660 X at4g33330 | 60 | 58 |
| AT3g18660 X at4g33340 | 60 | 46 |

Table 2 shows the percentage identity between AT3 g18660 and other glycogenin genes from Arabidopsis using the programme BESTFIT of the GCG package. In each case, the full length nucleotide and peptide was compared to the AT3g18660 gene.

These levels of identity are consistent with the genes encoding proteins with the same function. For example, the two yeast glycogenin genes are about 50% identical to one another at the protein level and are both known to be involved in the same pathway; both are essential for the production of glycogen and one can complement for the function of the other.

It is interesting that the carboxyl terminal region of the protein encoded by at1g77130 shows homology to a starch synthase (dull1) from maize. In yeast, glycogenin and glycogen synthase physically interact. This finding may be the first indication that a similar scenario exists in plants. The at1g77130 gene appears to be a duplication of the AT3g18660 sequence, and the small region of homology with dull1 may indicate that during the course of evolution this gene has become physically close to dull1. Recently published work (Yanai et al., 2001, Proc. Natl. Acad. Sci. USA 98(14): 7940-7945) suggests that a functional association between two genes can be derived from the existence of a fusion of the two as one continuous sequence in another genome. In yeast, it has been shown by experimentation that glycogenin and glycogen synthase physically interact and are associated together in an enzymatic complex to allow glycogen biosynthesis. The inventors believe that PGSIP interacts with soluble starch synthases at the start of the starch biosynthesis process. This could be the first step in the formation of a biosynthetic starch enzymes complex where PGSIP acts as a template, starch synthases extend the chain followed by branching by starch branching enzymes and other starch synthesis enzymes. It is likely that biosynthesis starch enzymes become associated with the very first complex formed in the process of the synthesis of a starch polymer.

The sequences of the six genes listed in Table 2 were compared by BLAST against the Arabidopsis sequences in an in-house database and a further hit was obtained. The identified sequence corresponding to SPTREMBL: Q8W4AZ, EMBL: AY062695 encodes a protein of 618 amino acids that showed strong homology to the glycogenin genes (4e-26). Further analysis of the sequence indicated that the protein represents the C terminal domain of the At1g77130 gene (080649, T14N5.1) and is also annotated as At1g77130, T14N5.1 which encodes an unknown protein. This sequence is set forth in SEQ ID NO: 23.

### Example 2

### Isolation of cDNA Encoding A. thaliana Glycogenin Homologue

Primers were designed to clone a full length cDNA representing the accession number AB026654, gene_id:MVE11.2 (at3g18660 (MIPS)) from an Arabidopsis thaliana cDNA pool. Sequencing the full length clone indicated that the gene encoded a protein of 659 amino-acids and consists of five exons. The cDNA sequence designated as SEQ ID NO: 2.

Arabidopsis thaliana was grown in growth cabinets with a 16 hours light and 8 hours dark period at a temperature of 22° C. during the day and 17° C. during the night. A mixed cDNA sample was made with total RNA from 10 different tissues mixed together in equal amounts: root, dividing cell culture, young leaf, mature leaf, stem, seedling, seed, flower buds+flowers, drought 6 days- and drought 10 days-subjected plants.

The primer used to make the first strand cDNA using Superscript II was from the original paper on PCR amplification by (Frohman et al. (1988) Proc. Natl. Acad. Sci. USA, 85:8998): 5′GACTCGAGTCGACATCGATTTTTTTTTTTTTTTTT 3′.

1 μl of this cDNA was used to amplify the cDNA clone representing the accession number GTD:S:1870408 (gene id:MVE11.2) utilizing the primers G1gfl and G1g int1 and C1aF and Glgstop2.

| | |
|---|---|
| G1gf1 primer: | 5′-GACCATGGCAAACTCTCCCGC-3′ |
| G1g int1 primer: | 5′-GCAGCATACTTTTCCAATTAC-3′ |
| C1aF primer: | 5′-GCAAGTTCCGGCTATGGCAGC-3′ |
| G1gstop2 primer: | 5′-GCGTCACAAGTTATGGCCGGG-3′ |

PCR Conditions:

Five 50 μl reaction was set up as follows:

| Composition | | PCR Programme | |
|---|---|---|---|
| Water | 35.5 μl | 95° C. | 2 min (hot start) |
| 10x buffer | 5 μl | 95° C. | 3 min |
| 4 mMdNTPs | 2.5 μl | 55° C. | 30 sec |
| Pfu Turbo polymerase | 1 μl | 72° C. | 2 min:30 sec |
| 4 mM primers | 5 μl | 72° C. | 10 min (extension) |
| cDNA | 1 μl | | |

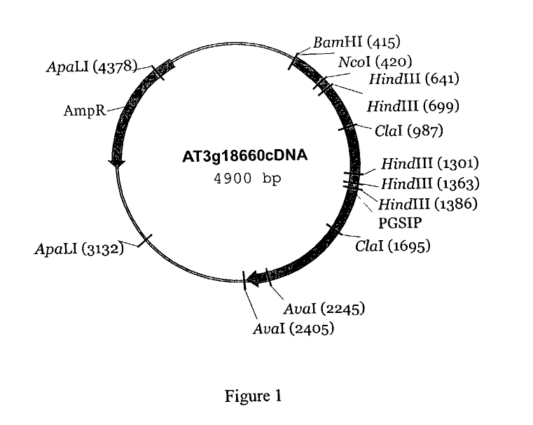

Two products were obtained. These were cloned in pBluescript vector (SK−) (Stratagene) and a full length clone was obtained. The map of this plasmid is shown in FIG. 1.

### Example 3

### Functional Analysis of the Arabidopsis cDNA

Yeast contains two glycogenin genes Glg1 (YKROS8w) and Glg2 (YJL137c). Double mutants in the above genes do not make any glycogen (Cheng et al (1995) Mol. and Cell Biology 15(12):6632-6640). Mutant yeast strains from the EUROSCARF (European Saccharomyces Cerevisiae ARchives For Functional Analysis) collection were obtained from SRD GmbH, D61440, Germany along with the wild type. Single mutants in the Glg1 and Glg2 genes were obtained in addition to the double mutant. Additionally a plasmid containing the entire Glg2 ORF including the promoter was also obtained. This plasmid was used as a positive control to establish a complementation assay. The description of the strains are:

Wild type

| ORF | Accession no. | Strain | Genotype |
|---|---|---|---|
| | Y00000 | BY4741 | MATa; his3Δ1; |
| | | | leu2Δ0; met15Δ0; |
| | | | ura3Δ0 |

Single Mutants:

| ORF | Accession no. | Strain | Genotype |
|---|---|---|---|
| YKR058W | Y15129 | G1G1 mutant | BY4742; Mat alpha; |
| | | | his3Δ1; leu2Δ0; |
| | | | ura3Δ0; |
| | | | YKR058w::kanMX4 |
| YJL137c | Y17003 | g1g2 mutant | BY4742; Mat a; his3 |
| | | | Δ1; leu2Δ0; ura3Δ0; |
| | | | YJL137c::kanMX4 |

Double Mutants:

| Mutant Strains | Genotype |
|---|---|
| 1. g1g1/g1g2 deleted | BY4742; Mat alpha; his3Δ1; leu2Δ0; ura3Δ0; |
| | YKR058w::kanMX4; YJL137c::kanMX4 |
| 2. g1g1/g1g2 deleted | BY4742; Mat a; his3Δ1; leu2Δ0; ura3Δ0; |
| | YKR058w::kanMX4; YJL137c::kanMX4 |

Plasmid

| Plasmid name | Gene | Marker |
|---|---|---|
| PYCG_YJL137c(pRS416) | G1g2ORF + prometer | URA3 |

Glycogen Defect Assay

First, it was established that the wild type and the double mutants were indeed different. For this experiment, freshly grown wild type, and the double mutants were picked up from YPD plates and the cells were suspended in 100 μl of water in an eppendorf tube. To this tube approximately 100 μl of glass beads (Sigma) and 10-20 μl of undiluted Lugol solution (Sigma) was added. The cells were vortexed briefly, spun down for few seconds and assayed for color development. The wild type cells stained blue whereas the double mutants did not stain and appeared white.

Complementation Assay

Double mutants were transformed with the plasmid pRS416 and the transformants were selected on CSM/Ura-plate (Uracil drop out plate). As a negative control, double mutants were transformed without the plasmid. Many colonies were obtained in the positive plate but no colonies were obtained from the negative control indicating that the transformation had worked. The transformed double mutants were grown overnight in CSM/Ura-liquid media along with wild type and single mutants. Next day OD₆₀₀ was checked to ensure equal amounts of cells in each of the tubes. Approximately equal amounts of cells were taken in an eppendorf tube and to this equal amounts of glass bead were added followed by 10-20 μl of undiluted Lugol solution (Sigma). The cells were vortexed briefly and centrifuged for few seconds and assayed for colour development. Complementation was observed in the double mutants as they appeared blue similar to the single g1g1 and g1g2 mutants.

Optimisation of the Assay to Distinguish Wildtype and Mutant Strains

A small amount of the wildtype (WT) and glycogenin double mutant (Mut) yeast strains were picked up from a well-grown plate, resuspended in 1 ml of water, and vortexed briefly. The cells were diluted further in 1 ml of water and 50 ul of the diluted cells were plated on YPD plates. The plate was incubated at 30° C. for two days and afterwards the plates were exposed to iodine vapour by inverting the plates on top of a 500 ml glass beaker containing iodine chips (Sigma) placed on a low heater under a fume cupboard briefly for 2-3 minutes. Afterwards the plates were left open in the fume cupboard briefly for 1 minute and the colour development was monitored. The WT cells stained brown and the double mutants (Mut) stained pale yellow.

Cloning PGSIP cDNA in into the pYES2 Vector for Complementation Studies

Two constructs were made to do the experiment, one contained the full length PGSIP cDNA including the transit peptide (TP) and another in which the transit peptide was removed (No transit peptide: NTP), these were cloned into pYes2 vector (Invitrogen). Primers were designed to amplify the full length PGSIP cDNA with the transit peptide (primers TPF and TPR) and without the transit peptide (primers NTPF and NTPR) so that these could be cloned into the pYes2 vector. A BamHI restriction enzyme site was incorporated into the forward primers (TPF and NTPR) and a XhoI restriction enzyme site was incorporated into the reverse primers (TPR and NTPR). The NTP forward primer (NTPF) was designed in such a manner so that it annealed at nucleotide position 190 of the full length PGSIP sequence and an ATG initiation codon was inserted after the BamHI site to ensure that translation into protein could occur. This resulted in a cDNA sequence lacking the first 63 amino acids of the PGSIP cDNA sequence which represents the transit peptide as predicted by the Target P program (Emanuelsson et al, J. Mol. Biol. 300:1005-1016 (2000). The primer sequences were as follows:

| | |
|---|---|
| TPF | 5′-GGATCCGACCATGGCAAACTCTCCCGC-3′ |
| TPR | 5′-CTCGAGGCGTCACAAGTTATGGCCGGG-3′ |
| NTPF | 5′-GGATCCATGTGTTGTTGTTTCACCAAG-3′ |
| NTPR | 5′-CTCGAGGCGTCACAAGTTATGGCCGGG-3′ |

A 50 ml PCR reaction was set up with Pfu polymerase (Stratagene) as follows: a coocktail solution was made with 35.5 ul water, 5 ul 10×PCR buffer+, 2.5 ul solution (20 mM MgCl and 4 mM dNTPs), 1 ul Pfu polymerase, 5 ul 4 mM primers (TP/NTP), and 1 ul cDNA (1/100 dil). The PCR thermocycler program consisted of a 95° C. 3 min (hot start), followed by 30 cycles of 95° C. for 30sec, 50° C. for 30sec, and 72° C. for 3 min. The final step in the program held the temperature at 24° C.

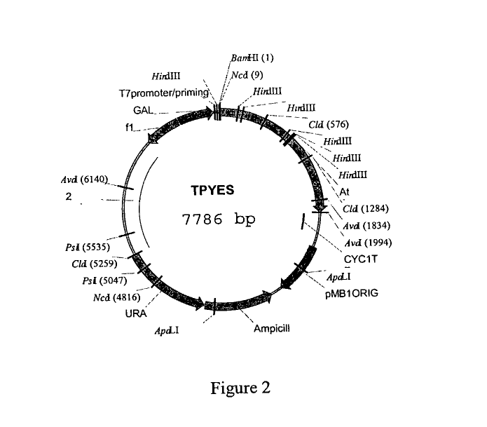

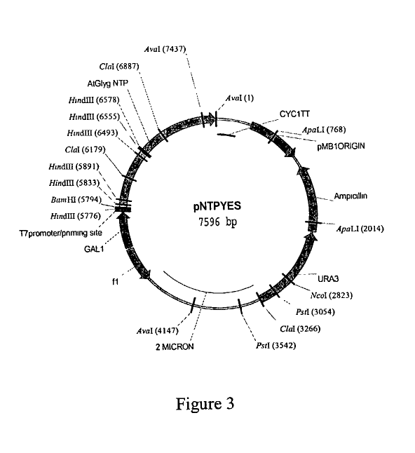

The amplified fragment was run out on an agarose gel, cut out and purified using the ‘Geneclean kit’ according to the manufacturers instructions (Bio101). The purified cDNA fragments were ligated into pBluescript vector (Stratagene) cut with EcoRV resttriction enzyme. Positive clones were identified and these were sequenced. Clones with the correct sequences were then cut with the restriction enzymes BamHI and XhoI and ligated in pYes2 vector cut with the restriction enzymes BamHI and XhoI. Positive clones were identified and these were named, pTPYes (FIG. 2) and pNTPYes (FIG. 3). In these plasmids, the cDNA was under the control of the yeast Gal 1 promoter that is both glucose repressible and galactose inducible.

Complementation Analysis with the Arabidopsis Glycogenin Gene

Yeast strains were transformed with the above plasmids following the method of Finley and Brent, 1995, (http://cmmg.biosci.wayne.edu/finlab/YTHprotocols.htm and links there in) in combination with the Clontech yeast transformation kit. From a freshly grown plate a 5 ml culture of yeast strain (WT and Mut) was inoculated in YPD medium (Clontech) overnight with shaking at 30° C. Next day, 3 ml freshly grown cells were inoculated into 150 ml YPD medium, (OD600=0.2) and grown shaking at 30° C. for 3-4 hours (OD600=0.7). 100 ml cells were then transferred to two 50 ml orange cap tubes and centrifuged at room temperature at 2000 rpm for 3 minutes. The supernatant was discarded completely. The cells were washed by resuspending them in 2.5 ml of sterile water followed by centrifugation as before. The supernatant was discarded and the cells were resuspended by adding 625 ul of Lithium Acetate (LiAc)/TE (10mM Tris HCL pH 7.5, 1 mM EDTA, 100 mM LiAc; made from a filter-sterile stock of IM LiAc, pH 7.5) in each tube. The cells were centrifuged as before and the supernatant was discarded. The cells were resuspended in 250 ml of LiAc/TE then pooled into a single eppendorf tube giving 500 ml of competent yeast cells. In an eppendorf tube the following was prepared, 6 ml Herring Testis DNA (Clontech,10 mg/ml, boiled earlier for 10 minutes and quenched on ice), 8 ml DNA [pYes2 empty plasmid, TPYes and NTP Yes DNA (˜2 ug)] and 6 ml of water making a total volume of 20 ml. In another tube 100 ml of competent yeast cells were added to which the 20 ml mixture made above, plus 11 ml DMSO and 600 ul of 40% PEG 4000 in LiAc/TE (made from stocks of IM LiAc pH 7.5, filter sterile 50% PEG 4000 in water, 1M Tris HCl pH 7.5 and 0.5M EDTA) was added. The tubes were inverted three to four times gently and incubated at 30° C. for 30 minutes. The tubes were inverted again gently and heat shocked at 42° C. for 20 minutes after which 50-100 ml was directly plated on CSM/Ura-/glucose plates. The plates were incubated for two to three days at 30° C. Additionally, as a negative control, WT and Mut yeast strains were transformed with the empty pYes2 plasmid. As a positive control the Mut strains were transformed with the yeast GLG2 gene (plasmid pRS416) purchased from EUROSCARF. The transformed cells were selected on CSM/Ura-glucose drop out plates. After two days the cells were picked individually into patches and streaked onto glucose and galactose plates. In the end, we had the following plates.(Table 4)

TABLE 4

| Name | Glucose | Galactose |
|---|---|---|
| 1. WT:pYes2 control | | Yes |
| 2. Mut:pYes2 control | Yes | Yes |
| 3. WT:NTP | Yes | Yes |
| 4. Mut:NTP | Yes | Yes |
| 5. WT:TP | Yes | Yes |
| 6. Mut:TP | Yes | Yes |
| 7. Mut:yeast GLG2 gene + | Yes | Yes |
| ve control | | |

Yeast strains used for the complementation experiment (Table 5)

TABLE 5

Name
1. WT:pYes2 control
2. Mut:pYes2 control
3. Mut:TP
4. Mut:NTP
5. Mut:yeastGLG2

The plates listed in Table 4 and Table 5 were grown for two days at 30° C. as described above. The cells were diluted and plated on to both CSM/Ura-glucose and CSM/Ura-galactose plates. After two days of growth at 30° C. the cells were exposed to iodine vapour as described above and photographs were taken. From the photographs, it was confirmed that the assay worked as the Mut strains containing the yeast GLG2 gene (no.7 from the table 4) stained brown both in the glucose and galactose plates. The WT strain (no.1 from the table 4) stained brown whereas the Mut strains (no. 2 from the table 4) containing the empty plasmid stained yellow. The cells containing the NTP plasmid (no. 4 from the table 4) stained yellow in glucose plate but it stained brown in galactose plates but the brown colour is not as intense as observed in Mut strains containing the yeast GLG2 gene indicating that the complementation is partial. This data indicates that the PGSIP cDNA is a functional orthologue of the yeast glycogenin gene and plays a role in starch biosynthesis especially in plants and particularly in Arabidopsis. The cells containing the TP plasmid (no. 3 from the table 4) stains yellow in glucose and galactose plates indicating that complementation was not achieved with this plasmid. In general, validating the function of plant genes by yeast complementation has been reported (Alderson et al, Proc Natl. Acad.Sci. USA, 88:8602-8605 (1991), Vogel et al., Plant J, 13 (5):673-683, 1998, Blazquez, et al., Plant J, 13 (5):685-689, 1998.

### Example 4

### cDNA Isolation from Maize Endosperm

Maize EST Identification

ESTs encoding corn glycogenin gene were identified using the program BLAST (Basic Local Alignment Search Tool; Altschul, S. F. et al (1990) J. Mol. Biol. 215:403-410, see also www.ncbi.nlm.nih.gov/BLAST/). A database search using the Arabidopsis gene AT3g18660 and at1g77130 against the maize database at NCBI identified accession no. GB: BF729544 and GB: BG837930 which showed significant similarity to the Arabidopsis glycogenin genes. The sequence of the two ESTs is shown in SEQ ID NO: 4, and SEQ ID NO: 5 respectively. A blastx analysis of the two ESTs against SPTREMBL database showed that EST BF729544 picked up the first hit to the AT3g18660 gene whereas EST BG837930 showed first hit to the at1g77130 gene. Protein alignments of these ESTs indicated that both ESTs were partial and they showed 85-86% identity to the above two Arabidopsis genes. Moreover, for EST BF729544 the identity was confined to the central portion of the AT3g18669 protein starting at amino-acid position 245 and ending at position 427, whereas for EST BG837930 the identity started at amino-acid position 391 and extending until position 632. A bestfit analysis between the two nucleotide sequences of the ESTs and the AT3g18660 gene showed that the two ESTs have 68-69% identity. A bestfit analysis between the two EST DNA sequences showed that there was a high degree of homology between the two ESTs. From the above analysis, it appears that EST BF729544 is the homolog of the Arabidopsis AT3g18660 gene, whereas EST BG837930 is a homolog of the Arabidopsis AT1g77130.

A database search using the Arabidopsis genes AT3g18660 and atg177130, against the maize database in-house identified four additional sequences which showed significant similarity to the Arabidopsis glycogenin genes. The four nucleotide sequences called Maize SEQ 1, Maize SEQ 2, Maize SEQ 3 and Maize SEQ 4 are shown in SEQ ID NOs: 27, 29, 31 and 33 and the deduced amino acid sequences for these nucleotide sequences are shown in SEQ ID NOs: 28, 30, 32 and 34.

Culture Conditions

Maize was grown in the greenhouse with a 16 hour daylight and 8 hour night period with a temperature of 24° C. during the day and 18° C. during the night. Seeds were harvested at different stages between 3 and 35 days after pollination (DAP). Young and medium leaves were also harvested.

Establishment of Copy Number and Identification of Glycogenin Homolog in Maize, Wheat and Arabidopsis

Genomic DNA was isolated from Arabidopsis, wheat and maize leaves according to the method of Davies et al., ((1994) Methods in Molecular Biology vol. 28: Protocols for nucleic acid analysis by non-radioactive probes, Isaac P. G. (ad) pp 9-15 Humana press, Totowa, N.J. USA). DNA was digested with restriction enzyme, EcoRI, XhoI and EcoRV and the digested DNA was run overnight at 20V in 1% agarose gels. The DNA was then transferred to a nylon membrane by vacuum blotting and two identical southern blots were prepared and each one was probed first at a high stringency and later at low stringency conditions. One blot was probed with a digioxygenin labelled AT3g18660 cDNA probe encoding the N-terminus of the gene (a 1.8 kb NcoI-AvaI fragment) and filter 2 was probed with AT3g18660 cDNA probe (PGSIP) encoding the C-terminus of the gene (a 700 bp C1a K fragment), FIG. 1. Hybridisation was done at 65° C. and the blots were first washed with 2×5 minutes with 2×SSC, 0.1×SDS and later with 0.1×SSC and 0.1×SDS at 65° C. (high stringency washes). Strong single bands of the expected sizes (5.9 kb in the XhoI cut DNA, 4.6 kb in the EcoRI cut DNA and 50.1 kb in the EcoRV cut DNA) were observed only in the lanes containing Arabidopsis DNA. No band was observed in the lanes containing maize and wheat DNA, as shown in FIG. 6. Later the blots were stripped and these were re-probed at 55° C. and washed at 60° C. for 2×15 minutes with 2×SSC, 0.5% SDS (low stringency washes). No band was observed in the lanes containing maize and wheat DNA. Later the blots were stripped and these were reprobed at 55° C. and washed at 60° C. for 2×15 minutes with 2×SSC, 0.5% SDS (low stringency washes). Three bands were observed in the lane containing XhoI digested Arabidopsis DNA, two-three bands were observed in the lanes containing maize and wheat DNA, as shown in FIG. 7. From the genomic sequence of the AT3 g18660 gene it was known that it spanned two Xho I, EcoR1 and EcoRV sites. This demonstrated that PGSIP exists as a gene family comprising of about 2-3 genes in Arabidopsis, maize and wheat.

RNA Extraction and First Strand cDNA Synthesis

Total RNA was extracted from the tissues described above using the method of Napoli et al (1990), Plant Cell, 2, 279-289 and in some cases using Qiagen RNA extraction kit following manufacturer s protocol. First strand cDNA was made using SuperscriptII everse transcriptase (GIBCO-BRL) and oligo dT primer as described in (Frohman et al, 1988), Proc. Natl. Acad. Sci. USA, 85:8998):

5′ GACTCGAGTCGACATCGATTTTTTTTTTTTTTTTT 3′.

This cDNA pool was used to amplify a maize cDNA homolog to the Arabidopsis lycogenin gene (AT3g18660 and at1g77130) utilising the sequence information from the STs, GB:BF729544 and GB: BG837930 described above.

EST BF729544 and BG837930 overlapped and these were combined to deduce a ingle maize PGSIP sequence. Primers were designed to amplify a maize cDNA clone orresponding to this sequence. Primer sequences were as follows.

| | |
|---|---|
| [GigmaF] | 5′-GGCAATAGAGGAATTCATGTGC-3′ |
| [GlgmaR] | 5′-CGTGCAGAACTCGGACCACAG-3′ |

Construction of a Maize cDNA Library

Total RNA was extracted from the various tissues described above (leaves and seeds ranging from 3-35 DAP). The RNA obtained was mixed in equal amounts. This RNA mixture was then used to make a maize cDNA library using SMART cDNA library construction kit (Clontech) following manufacturer's instruction.

Cloning of Maize cDNA

1 ul of this first strand cDNA obtained above was used to amplify the cDNA clone represented by the ESTs by PCR using the primers GlgmaF and GlgmaR, the PCR product obtained was cloned into EcoRV cut pBlueScript (SK−) and positive clones were identified. These positive clones were sequenced to confirm that the product obtained indeed represented the sequence in the EST accession number, BF729544. This product was then used to screen the cDNA library and a full length clone was obtained. Similarly a cDNA clone represented by the EST accession no. BG837930 was also cloned.

The PCR conditions were the same as described before for cloning the Arabidopsis gene (AT3gl 8660) of SEQ ID NO: 2.

### Example 5

### cDNA Isolation from Wheat Endosperm

A database search using the Arabidopsis genes AT3g18660 and at1g77130, against the wheat in-house database identified one sequence, which showed significant similarity to the Arabidopsis PGSIP genes (e-137). The sequence called Wheat SEQ1 is shown in SEQ ID NO: 20.

Culture Conditions

Wheat variety NB1 (described in patent WO 00/63398) was grown in the glass house with a 16 hour daylight and 8 hour night period with 22° C. during the day and 15° C. during the night. Seeds were harvested at different stages between 5 and 20 days after pollination (DAP). Young and medium leaves were also harvested.

RNA Extraction and First Strand cDNA Synthesis

Total RNA was extracted from the above tissues using the method of Napoli et al (1990) and in some cases using Qiagen RNA extraction kit following manufacturer's protocol. First strand cDNA was made using SuperscriptII reverse transcriptase (GIBCO-BRL) and oligo dT primer as described in (Frohman et al, (1988), Proc. Natl. Acad. Sci. USA, 85:8998. This cDNA pool was used to amplify a wheat cDNA homolog to the Arabidopsis glycogenin gene (AT3g18660 and at1g77130) utilising the sequence information from the maize ESTs, NCBI accession no. BF729544 and BG837930 described above.

Wheat cDNA Library Making

Total RNA was extracted from the various tissues described above (leaves and seeds ranging from 7-30 days post anthesis (DPA). The RNA obtained was mixed in equal amounts. This RNA mixture was then used to make a wheat cDNA library using SMART cDNA library construction kit (Clontech). Additionally a genomic library from Triticum tauschii, var strangulata, accession number CPI 110799, described in (Rahman et al., 1997, Genome, 40:465-474) was also used in this study. The cDNA library from Wheat cv Wyuna described in (Li et al., 1999, Theor. Appl. Gen. 98:226-233) was also used in this study.

Cloning of Wheat cDNA

Because a strong band was observed on southern blots probed with the Arabidopsis gene (AT3g18660), it was assumed that there is significant degree of homology between the Arabidopsis, maize and wheat DNA sequences. A comparison of the Arabidopsis and the maize EST sequences also suggested that this was the case. A wheat cDNA library was screened with probes made from the maize and the Arabidopsis glycogenin gene. A full length clone was obtained by restriction mapping and analysing the sequence of a number of positive clones.

PCR Conditions

The PCR conditions were the same as described before for cloning the Arabidopsis gene (AT3g18660).

### Example 6

### Agrobacterium Constructs

Construct Making

The pSB111 Sulugi described in patent publication WO 00/63398 was used. Six different constructs were made, one each for maize, wheat and Arabidopsis in sense orientation and one each for maize, wheat and Arabidopsis in antisense orientation for constitutive expression. Another six set of constructs, were also made using seed specific promoters.

Two constructs were made, one for overexpression and another for downregulation of the Atglycogenin gene. For overexpression, the Atglycogenin gene was excised out from the plasmid (At3g18660 (PGSIP), FIG. 1) with SalI-EcoRI digest and ligated in SalI-EcoRI cut pJIT65 resulting in plasmid pCL68. This plasmid was then digested with EcoRI-XhoI and the fragment was ligated into SalI-Smal cut Nos-NptII SCV resulting in plasmid pCL68 SCV. In this plasmid the Atglycogenin is under 2×35S promoter for constitutive expression.

For RNAi construct, first a fragment representing the 3′ end of the Atglycogenin gene was amplified by PCR using ClaF and Glgstop2 primer (see example 2) and was cloned into pBluescript. The resulting construct was designated pMC167. Clones in both orientation were obtained and the clone with the fragment in reverse orientation was called pMC167inv. pMC167inv was cut with EcoRV-SmaI and ligated back resulting in plasmid pMC167del. pMC167del was cut with HindII-BamHI and ligated into HindIII-BamHI cut pT7blue2 resulting in plasmid “GlycoinpT7Blue2” (pCL66). Another plasmid (called GlycogeninIRstep 1, pCL67) was created by cutting pMC 167inv with XhoI-EcoRV and ligating this fragment into XhoI-EcoRV cut pWP446A containing the AtSac25 intron1. Finally, plasmid “GlycoinpT7Blue2”, pCL66 was cut with BamHI-SstI and the fragment ligated into BamHI-SstI cut “GlycogeninIRstepl”, pCL67 resulting in plasmid pCL69. pCL69 was cut with EcoRI-XhoI and the fragment was ligated in SCV Nos-NptII at the SmaI-SalI site resulting in plasmid pCL76 SCV. In this plasmid the At glycogenin (PGSIP) RNAi is under 2×35S promoter for constitutive expression.

FIG. 6 summarises the whole process and the maps of these plasmids are shown in FIGS. 9 and 10. The plasmids were transformed into the GV3101 Agrobacterium strain and the Arabidopsis plants were transformed.

### Example 7

### Transformation of Wheat

Wheat plants transformed with the constructs of Example 6 were produced by the seed inoculation method described in patent publication WO 00/63398. Solanum tuberosum c.v. Prairie was transformed with pCL68 SCV and pCL76 SCV using the method of leaf disk cocultivation essentially as described by Horsch et al. (Science 227: 1229-1231, 1985). The youngest two fully-expanded leaves from a 5-6 week old soil grown potato plant were excised and surface sterilized by immersing the leaves in 8% ‘Domestos’ for 10 minutes. The leaves were then rinsed four times in sterile distilled water. Discs were cut from along the lateral vein of the leaves using a No.6 cork borer. The discs were placed in a suspension ofAgrobacterium tumefaciens strain LBA4404 containing one of the two plasmids listed above for approximately 2 minutes. The leaf discs were removed from the suspension, blotted dry and placed on petri dishes (10 leaf discs/plate) containing callusing medium (Murashige and Skoog agar containing 2.5 μg/ml BAP, 1 μg/ml dimethylaminopurine, 3% (w/v) glucose). After 2 days the discs were transferred onto callusing medium containing 500 μg/ml Claforan and 50 μg/ml Kanamycin. After a further 7 days the discs were transferred (5 leaf discs/plate) to shoot regeneration medium consisting of Murashige and Skoog agar containing 2.5 μg/ml BAP, 10 μg/ml GA3, 500 μg/ml Claforan, 50 μg/ml Kanamycin and 3% (w/v) glucose. The discs were transferred to fresh shoot regeneration media every 14 days until shoots appeared. The callus and shoots were excised and placed in liquid Murashige and Skoog medium containing 500 μg/ml Claforan and 3% (w/v) glucose. Rooted plants were weaned into soil and grown up under greenhouse conditions to provide tuber material for analysis.

Alternatively, microtubers were produced bytaking nodal pieces oftissue culture grown plants onto Murashige and Skoog agar containing 2.5 μg/ml Kanamycin and 6% (w/v) sucrose. These were placed in the dark at 19° C. for 4-6 weeks when microtubers were produced in the leaf axils.

### Example 8

### Transformation of Maize

Maize plants transformed with the constructs of Example 6 were produced by the seed inoculation method described in patent publication WO 00/63398.

### Example 9

### Transformation of Potato

Transgenic potato plants expressing the Arabidopsis plant glycogenin-like gene in sense and antisense orientation were produced.

### Example 10

### Characterisation of the Transgenic Lines

Transgenic plants were analysed by the following methods For sense constructs, 20 T1 lines were analysed; for antisense constructs, 50 T1 lines were analysed. Plants transformed with sense and antisense sequences of the invention were observed to have altered starch synthesizing ability which was linked to the expression of the transgene.

For the maize, wheat, and potato lines examined, several techniques of analysis were employed. PCR-positive line identification, northern-RNA expression, southern-copynumber detection, western-protein expression, amylogenin activity, starch structure and quality, and phenotype all confirmed the successful transformation of the maize, wheat, and potato.

### Example 11

### cDNA Isolation from Rice

The six genes listed in Table 2 were blasted against the rice sequences collected in an in-house database and one new hit was obtained. The accession corresponded to SPTREMBL:Q94HG3, EMBL:AC079633 (SEQ. ID NO; 25) which encodes a protein of 614 AA and shows strong homology to the PGSIP gene (e-129).

### Example 12

### Arabidopsis Transformation

Arabidopsis thaliana c.v. Columbia plants were transformed according to the method of Clough and Brent 1998 Plant J. 16(6):735-743 (1998) with slight modification. Plants were grown to a stage at which bolts were just emerging. Phytagar 0.1% was added to the seeds and these were vernalized overnight at 4° C. We used 10-15 seeds per 3×5 inch pots. Seed was added onto the soil with a pipette, about 4-5 seeds per ml was dispersed. Seeds were germinated as usual (ie under humidity pots were covered until first leaves appeared and then over a two day period the lid was cracked and then removed). Plants were grown for about 4 weeks in the greenhouse (long day condition) until bolts emerged. The first bolts were cut to encourage growth of multiple secondary bolts. Bolts containing many unopened flower buds were chosen for dipping.

Growing the Agrobacterium Culture

Aliquots of the Agrobacterium strain GV3101 carrying the constructs pCL68 SCV and CL6876 SCV were grown first as a 5 ml culture in YEP containing Gentamycin (15 ug/ml) and anamycin 20 ug/ml. Next day, 2 ml freshly grown culture was added to 400 ml YEP media (10 g Yeast Extract, 10 g peptone, 5 g NaCl, pH 7.0) in a 2 litre flask and the flask was incubated at 28° C. incubator with shaking overnight. Next day OD 600 of the cells was measured and found to be 1.8. Cells were divided into 2× Oakridge bottles and harvested by centrifugation at 500Orpm for 10 min in a GSA rotor at room temperature The pellet was resuspended in 3 volumes of infiltration media so that the final concentration of the culture was 0.6. Infiltration media was prepared by adding the following. ½ Murashige and Skoog Salts, 1× Gamborg's Vitamins and 0.44 uM Benzylamino Purine (10 ul per L of a 1 mg/ml stock), pH was adjusted to 5.7 with NaOH. Then 0.02% Silwet (200 ul per 1 L) was added and mixed into the solution.

Arabidopsis Transformation by Dipping

500 ml of resuspended Agrobacterium was poured into a tray and plants were inverted into Agrobacterium solution in batches of 10 for 15 minutes. After 15 minutes the plants were lifted and the excess solution drained, The plants were transferred on their sides to a fresh tray containing tissue paper to allow further soaking of the solution and then transferred to propagating trays. The plants were immediately covered with lids to maintain humidity. After two days the lid was removed and the plants allowed to grow normally. They were not watered for one week until the soil looked dry. After flowereing was complete and the siliques on the plants were dry, all the seeds from one pot were harvested. The seeds were completely dried by keeping harvested seed in an envelope for one week

### Example 13

### Selection of Transformed Arabidopsis thaliana Seed

Seed produced from transformed Arabidopsis thaliana c.v. Columbia plants was weighed into 10 mg aliquots, equivalent to about 500 individual seed, and placed into a sterile 15 ml tube. The seed was surface sterilised by treating with 10 ml of Teepol bleach/Tween 20 solution (500 ml of 50% (v/v) Teepol bleach containing 1 drop of Tween 20) for five minutes. The seeds were then washed four times with 10 ml Tween 20 in sterile water (1 drop Tween 20 in 500 ml sterile water). The seeds were then suspended in 5 ml sterile water and 5 ml warm 0.5% agar, mixed carefully and then half of the seeds were spread over one petri dish containing half strength Murashige and Skoog agar medium and the other half over a second dish containing half strength Murashige and Skoog agar medium plus 50 μg/ml kanamycin. The plates were sealed and incubated at 4° C. for 48hours. The plates were then transferred to a growth room under low light (2000 lux). Seed on both types of plate germinated but on the plates containing kanamycin non-resistant plants bleached and died within 7 days. FIG. 8 demonstrates this selection of kanamycin resistant seedlings. After 14 days the resistant plants were transferred from the selective medium onto MS medium for a further 10 days before being transferred into soil. The plants were grown on to produce leaf material for further analysis.

### Example 14

### Analysis of Arabidopsis thaliana Plants Transformed with pCL68 SCV for the Presence of the PGSIP Construct

For the pCL68 SCV transformed lines a total of 31 kanamycin resistant plants were obtained from four of the original floral dips. These were tested for the presence of the construct by PCR.

Genomic DNA Extraction

Leafmaterial was taken from regenerated Arabidopsis thaliana plants transformed with pCL68 SCV and genomic DNA isolated. One leaf was excised from a plant growing in soil and placed in a 1.5 ml eppendorf tube. The tissue was homogenised using a micropestle and 400 μl extraction buffer (200 mM Tris HCL pH 8.0; 250 mM NaCl; 25 mM EDTA; 0.5% SDS) was added and ground again carefully to ensure thorough mixing. Samples were vortex mixed for approximately 5 seconds and then centrifuged at 10,000 rpm for 5 minutes. A 350 μl aliquot of the resulting supernatant was placed in a fresh eppendorf tube and 350 μl chloroform was added. After mixing, the sample was allowed to stand for 5 minutes. This was then centrifuged at 10,000 rpm for 5 minutes. A 300 μl aliquot of the supernatant was removed into a fresh eppendorf tube. To this was added 300 μl of propan-2-ol and mixed by inverting the eppendorf several times. The sample was allowed to stand for 10 minutes. The precipitated DNA was collected by centrifuging at 10,000 rpm for 10 minutes. The supernatant was discarded and the pellet air dried. The pellet of DNA was resuspended in 50 μl of distilled water and was used as a template in PCR.

PCR Detection of PGSIP

A pair of optimised oligonucleotide primers were designed and synthesised to enable the detection of the pCL68 SCV construct in transformed plants. The sequences of these primers were:

| | |
|---|---|
| ATGLY002: | CGTCTCGTGTCTGGTTTATATTCA |
| ATGLY003: | TCGATGCCTGAGATCTCAGCT |

PCR mixtures which contained 5 μl 10× Advantage Taq buffer; 5 μl 2 mM dNTPs; 0.5 μl of primer ATGLY002 (100 μM); 0.5 μl of primer ATGLY003 (100 μM); 5 μl DNA template Arabidopsis thaliana genomic DNA or control pCL68 SCV plasmid DNA); 0.25 μl Advantage aq polymerase; 33.75 pi distilled water in a final volume of 50 μl were set up. The PCR was carried out on a thermocycler using the following parameters: first a hot start at 94° C. for 5 min, then 25 cycles consistingof94° C. for 15 sec, 55° C. for 30 sec, and 72° C. for 3 min. The cycles were followed by 72° C. for 5 min and a final step of holding the samples at 8° C.

A diagnostic DNA fragment of 977 bp was produced in these reactions. Results of the PCR are shown in FIG. 11.

The PCR results for pCL68 SCV transformed plants indicated that of the 30 of the 31 of the plants examined had successfully been transformed. Thus, all of the plants except for the plant labeled 1-005 contained the PGSIP gene.

### Example 15

### Analysis of Arabidopsis thaliana Plants Transformed with pCL76 SCV for the Presence of the PGSIP Downregulation Construct

For the pCL76 SCV transformed lines a total of 10 kanamycin resistant plants were obtained. Leaf material was taken from regenerated Arabidopsis thaliana plants transformed with pCL76 and genomic DNA isolated. One leaf was excised from a plant growing in soil and placed in a 1.5 ml eppendorf tube. The tissue was homogenised using a micropestle and 400 μl extraction buffer (200 mM Tris HCL pH 8.0; 250 mM NaCl; 25 mM EDTA; 0.5% SDS) was added and ground again carefully to ensure thorough mixing. Samples were vortex mixed for approximately 5 seconds and then centrifuged at 10,000 rpm for 5 minutes. A 350 μl aliquot of the resulting supernatant was placed in a fresh eppendorf tube and 350 μl chloroform was added. After mixing, the sample was allowed to stand for 5 minutes. This was then centrifuged at 10,000 rpm for 5 minutes. A 300 μl aliquot of the supernatant was removed into a fresh eppendorf tube. To this was added 300 μl of propan-2-ol and mixed by inverting the eppendorf several times. The sample was allowed to stand for 10 minutes. The precipitated DNA was collected by centrifuging at 10,000 rpm for 10 minutes. The supernatant was discarded and the pellet air dried. The pellet of DNA was resuspended in 50 μl of distilled water and was used as a template in PCR.

PCR Detection of PGSIP RNAi DNA

A pair of optimised oligonucleotide primers were designed and synthesised to enable the detection of the pCL76 SCV construct in transformed plants. The sequences of these primers were:

| | |
|---|---|
| ATGLY001: | TTTGAACAAACAAAAAGGTGGAAC |
| ATGLY002: | CGTCTCGTGTCTGGTTTATATTCA |

PCR mixtures which contained 5 ml 10× Advantage Taq buffer; 5 ml 2 mM dNTPs; 0.5 ml of primer ATGLY001 (100 mM); 0.5 ml of primer ATGLY002 (100 mM); 5 ml DNA template (Arabidopsis thaliana genomic DNA or control pCL76 SCV plasmid DNA); 0.25 ml Advantage Taq polymerase; 33.75 ml distilled water in a final volume of 50 ml were set up. The PCR was carried out on a thermocycler using the following parameters: first a hot start at 94° C. for 5 min, then 25 cycles of 94° C. for 15 sec, 55° C. for 30 sec, and 72° C. for 3 min. The cycles are followed by 72° C. for 5 min and the samples are then held at 8° C.

A diagnostic DNA fragment of 819 bp was produced in these reactions. See FIG. 13. Out of 8 kanamycin resistant plants tested, 2 were shown to contain the PGSIP RNAi gene construct.

### Example 16

### Constitutive Overexpression and Downregulation of PGSIP Gene in Barley

Starch is made in the leaves and the grain. To test the effect of overexpressing and downregulating the PGSIP gene in a monocot species, plasmids pCL68 SCV (sense construct) and pCL76 SCV (RNAi construct) were expressed in barley. These plasmids conferred constitutive expression as the genes were under the control of the double 35S promoter. Additionally, the full length gene and the RNAi cassette were expressed under the control of the rice actin promoter (U.S. Pat. No. 5,614,1876). For this purpose, the Gateway cloning technology was used according to manufacturers instruction with slight modification (Invitrogen). The full length PGSIP was excised from plasmid pMC168 with NcoI-EcoRI and cloned into pENTR4 vector cut with NcoI-EcoRI resulting in plasmid called pMC175. The RNAi cassette was excised from plasmid pCL76 SCV with SalI-EcoICR1 and cloned into pENTR1 vector cut with SalI-EcoRV resulting in plasmid pMC174. These plasmids were then recombined with Destination vector pWP492R12 SCV that contained the actin promoter flanked by two recombination sites (attR1 and attR2 on either side (Invitrogen). This resulted in plasmids pMC 177 and pMC 176 respectively which contained the PGSIP gene and the RNAi construct under the control of the rice actin promoter (U.S. Pat. No. 5,614,1876). These plasmids are shown in FIGS. 14 and 15.

The constructs were transformed into Agrobacterium strain (AGL-1) (Lazo et al., 1991, Bio/Technol 9: 963-967) for barley transformation. Immature embryos of the barley variety Golden Promise were transformed essentially according to the method of Tingay et al. (The Plant Journal 11(6): 1369-1376, 1997). Donor plants of Golden Promise were grown with an 18 hours day, and 18/13° C. Immature embryos (1.5-2.0 mm) were isolated and the axes removed. They were then dipped into an overnight liquid culture of Agrobacterium, blotted and transferred to co-cultivation medium. After 2 days the embryos were transferred to MS based callus induction medium with Asulam and Timentin for 10 days. Tissues were transferred at 2 weekly intervals, and at each transfer they were cut into small pieces and lined out on the plate. At the third transfer, only the embryogenic tissue was moved on to fresh medium. After a total of 8 weeks in culture, the tissue was transferred to regeneration medium (FHG), where plantlets formed within 2-4 weeks. These were transferred to Beatsons glass jar with growth regulator free medium until roots had formed, when they were transferred to Jiffies expandable peat pellets and then to the Conviron growth chamber.

The plants were analysed by PCR using following primers.

For plants containing pCL68 plasmid (sense expression)

| | |
|---|---|
| 5-′ATTTGGAGAGGACAGCCCAAGC | Glyc For |
| 5′-CTCCATCGTTGGATCTCGTTCG-3′ | Glyc Rev (S) |

For plants containing pCL76 plasmid (RNAi expression)

| | |
|---|---|
| 5′-ATTTGGAGAGGACAGCCCAAGC-3′ | Glyc For |
| 5′-GCGTCATCTTCATCGCCAATCC-3′ | Glyc Rev (D) |

PCR was carried out as described in above

Results:

Six barley plants were regenerated after transformation with plasmid pCL68 SCV and eight plants with plasmid pCL76 SCV. The plants were first analysed by PCR and the leaves of the positive plants were subjected to iodine staining by Lugol. The results of PCR analysis are presented in Table 7.

TABLE 7
results of PCR screen of barley plants transformed with pCL68 SCV
or pCL76 SCV.

| Construct | Plant no | PCR no. | PCR |
|---|---|---|---|
| | Control1 | GG11 | Neg |
| | Control2 | GG12 | Neg |
| | Control3 | GG13 | Neg |
| pCL68 | 1 | GG1 | Pos |
| pCL68 | 2 | GG2 | Neg |
| pCL68 | 3 | | |
| pCL68 | 4.1 | GG8 | Neg |
| pCL68 | 5.1 | | |
| pCL68 | 6.1 | GG3 | Neg |
| pCL68 | 6.2 | | |
| pCL68 | 6.3 | GG9 | Neg |
| pCL68 | 7.1 | GG10 | Neg |
| pCL76 | 1.1 | GG4 | Pos |
| pCL76 | 1.2 | GG5 | Pos |
| pCL76 | 1.3 | GG6 | Pos |
| pCL76 | 1.4 | GG14 | Pos |
| pCL76 | 1.5 | GG15 | Neg |
| pCL76 | 2 | GG7 | Neg |
| pCL76 | 3.1 | GG16 | Pos |
| pCL76 | 4.1 | GG17 | Neg |

One plant containing the sense construct was found to contain more starch granules in its leaves relative to control plants without the sense construct. The plants containing the RNAi construct were found to lack starch granules as shown in FIG. 17.

### Example 17

### Seed Specific Overexpression and Downregulation of the PGSIP Gene in Barley

For seed specific expression, the plasmids pMC174 and pMC175 were recombined with the plasmid pWP491R12SCV that contained the seed specific promoter flanked by two recombination sites (attR1 and attR2 on either side (Invitrogen)). Barley plants were transformed according to the method of Tingay et al. (1997) with some modification as described for Example 13.

### Example 18

### Analysis of Transformed Solanum tuberosum Plants for Presence of the PGSIP Construct

Analysis of Regenerated Potato transformants.

Leaf material was taken from regenerated potato plants and genomic DNA isolated.

One large potato leaf (approximately 30 mg) was excised from an in vitro grown plant and placed in a 1.5 ml eppendorf tube. The tissue was homogenised using a micropestle and 400 μl extraction buffer (200 mM Tris HCL pH 8.0; 250 mM NaCl; 25 mM EDTA; 0.5% SDS) was added and ground again carefully to ensure thorough mixing. Samples were vortex mixed for approximately 5 seconds and then centrifuged at 10,000 rpm for 5 minutes. A 350 μl aliquot of the resulting supernatant was placed in a fresh eppendorf tube and 350 μl chloroform was added. After mixing, the sample was allowed to stand for 5 minutes. This was then centrifuged at 10,000 rpm for 5 minutes. A 300 μl aliquot of the supernatant was removed into a fresh eppendorf tube. To this was added 300 μl of propan-2-ol and mixed by inverting the eppendorf several times. The sample was allowed to stand for 10 minutes. The precipitated DNA was collected by centrifuging at 10,000 rpm for 10 minutes. The supernatant was discarded and the pellet air dried. The pellet of DNA was resuspended in 50 μl of distilled water and was used as a template in PCR.

PCR mixtures which contained 5 μl 10× Advantage Taq buffer; 5 μl 2 mM dNTPs; 0.5 μl of either primer ATGLY001 or ATGLY003 (100 μM); 0.5 μl of primer ATGLY002 (100 μM); 5 μl DNA template (Solanum tuberosum c.v. Prairie genomic DNA, control pCL68 SCV plasmid DNA or control pCL76 SCV plasmid DNA); 0.25 μl Advantage Taq polymerase; 33.75 μl distilled water in a final volume of 50 μl were set up. The PCR was carried out on a thermocycler using the following parameters: first a hot start at 94° C. for 5 min, followed by 25 cycles of 94° C. for 15 sec, 55° C. for 30 sec, and 72° C. for 3 min. The cycles were followed by 72° C. for 5 min and a finally holding the temperature at 8° C.

A diagnostic DNA fragment of 977 bp was produced in these reactions from plasmid pCL68 SCV or 819 bp from plasmid pCL76 SCV. Lines of Solanum tuberosum c.v. Prairie transformed with pCL68 SCV or pCL76 SCV were tested by PCR and were shown to contain the construct.

FIG. 16 shows the results for pCL68 SCV and FIG. 17 shows the results for pCL76 SCV. Of 18 plants transformed with pCL68 SCV, all 18 contained the sense PGSIP construct. For the PGSIP RNAi construct (pCL76 SCV), 3 out of 8 plants contained the construct.

### Example 19

### Analysis of Transformed Plants for PGSIP Expression

Raising Antisera to PGSIP Proteins.

Expression of PGSIP proteins can be analysed by Western blotting. Antibodies to PGSIP are raised by inoculating rabbits with peptides corresponding to the Arabidopsis thaliana PGSIP protein sequences produced by expressing the sequence as a transcriptional fusion with glutathione-S-transferase in E. coli cells

Preparation of Protein Extracts.

Protein extracts from potato tuber were produced by taking up to 100 mg of tissue and homogenising in 1 ml of ice cold extraction buffer consisting of 50 mM HEPES pH 7.5, 10 mM EDTA, 10 mM DTT. Additionally, protease inhibitors, such as PMSF or pepstatin were included to limit the rate of protein degradation. The extract was centrifuged at 13000 rpm for 1 minute and the supernatant decanted into a fresh eppendorf tube and stored on ice. The supernatants was assayed for soluble protein content using, for example, the BioRad dye-binding protein assay (Bradford, M. C. (1976) Anal. Biochem. 72, 248-254).

An aliquot of the soluble protein sample, containing between 10-50%g total protein was placed in an eppendorf tube and excess acetone (ca 1.5 ml) added to precipitate the proteins which were collected by centrifuging the sample at 13000 rpm for 5 minutes. The acetone was decanted and the samples air-dried until all the residual acetone has evaporated.

SDS-Polyacrylamide Gel Electrophoresis.

The protein samples were separated by SDS-PAGE. SDS PAGE loading buffer (2% (w/v) SDS; 12% (w/v) glycerol; 50 mM Tris-HCl pH 8.5; 5 mM DTT; 0.01% Serva blue G250) was added to the protein samples (up to 50 μl). Samples were heated at 70° C. for 10 minutes before loading onto a NuPage polyacrylamide gel. The electrophoresis conditions were 200 V constant for 1 hour on a 10% Bis-Tris precast polyacrylamide gel, using 50 mM MOPS, 50 mM Tris, 1 mM EDTA, 3.5 mM SDS, pH 7.7 running buffer, according to the NuPage methods (Invitrogen, U.S. Pat. No. 5,578,180).

Electroblotting.

Separated proteins were transferred from the acrylamide gel onto PVDF membrane by electroblotting (Transfer buffer: 20% methanol; 25 mM Bicine pH 7.2; 25 mM Bis-Tris, 1 mM EDTA, 50 μM chlorobutanol) in a Novex blotting apparatus at 30 V for 1.5 hours.

Immunodetection.

After blocking the membrane with 5% milk powder in Tris buffered saline (TBS-Tween) (20 mM Tris, pH 7.6; 140 mM NaCl; 0.1% (v/v) Tween-20), the membrane was challenged with a rabbit anti-PGSIP antiserum at a suitable dilution in TBS-Tween. Specific cross-reacting proteins were detected using an anti-rabbit IgG-Horse radish peroxidase conjugate secondary antibody and visualised using the enhanced chemiluminescence (ECL) reaction (Amersham Pharmacia).

Detection of mRNA.

Expression of PGSIP mRNA was analysed in plants by rtPCR or by Northern blotting.

### Example 20

### Analysis of Leaf Starch Content

Samples of leaves from control and transformed Arabidopsis thaliana plants which had been grown for 24 hours under high light (about 60 mg) were taken in a microfuge tube and extracted with 100 μl of 45% HClO₄. This suspension was diluted with 1 ml of distilled water and centrifuged (14000 rpm, 2 min.) Aliquots of the extracts were then analysed for starch content by taking 100 μl of the extract and mixing with an equal volume of Lugol's solution, the optical density of which was then measured at 540 nm using a microplate reader. Standard starch mixtures were prepared in the same way and measured at the same time and the starch content of the extracts was calculated by reference to these standards.

TABLE 8
Starch contents of leaves of Arabidopsis thaliana plants transformed
with pCL68 SCV (sense construct comprising SEQ ID NO: 1) compared
with the starch contents of leaves of non transformed (ncc) control
plants. Control value is the mean ± (the standard error of the mean)
for three plants.

| | leaf starch content ug/g fresh |
|---|---|
| samples | weight (FWt). |
| 37256 | 19.95 |
| 1-002 | 12.68 |
| 1-003 | 49.68 |
| 1-004 | 48.02 |
| 1-005 | 13.88 |
| 37407 | 17.47 |
| 37437 | 49.55 |
| 37468 | 24.88 |
| 37499 | 8.65 |
| 37529 | 17.71 |
| 37560 | 15.93 |
| 37590 | 9.95 |
| 37621 | 6.02 |
| 37257 | 21.9 |
| 37288 | 18.20 |
| 37316 | 11.82 |
| 37261 | 22.85 |
| 37381 | 9.51 |
| 37412 | 13.21 |
| 37442 | 33.60 |
| 37473 | 17.96 |
| 37504 | 8.88 |
| 37534 | 18.58 |
| 37565 | 11.98 |
| 37295 | 32.83 |
| 37323 | 38.43 |
| 37354 | 16.16 |
| ncc | 22.59 |
| | (±5.08) |

The ncc value represents the mean and standard error for the three control plants. Each data point otherwise represents a single leaf from an individual plant. Taking the error of the control as a measure of the population variation, then plants 1-003, 1-004, 1-007, 1-008, 6-007 and 9-003 have significantly more starch in their leaves than the controls. Plants 1-009, 1-012, 1-013, 2-003, 6-005, 6-009 and 6-011 have significantly lower starch contents. The copy number and level of expression of the sense construct in the plants are to be determined. The results demonstrate that a sense construct comprising SEQ ID NO: 1 can effectively alter the content of starch.

TABLE 9
Starch contents of leaves of Arabidopsis thaliana plants
transformed with pCL76 SCV (RNAi construct) compared to controls.

| | | starch content |
|---|---|---|
| Samples | | μg per leaf |
| pCL76 SCV | 7 | 27.20 |
| pCL76 SCV | 20.1 | 26.96 |
| Control | ncc | 42.97 |

The data in these tables shows that the leaves of the transformed plants have an altered starch content compared to the untransformed controls (ncc).

### Example 21

### Microscopic Analysis of Starch Granule Size and Number

Starch granules were extracted from Arabidopsis thaliana or Solanum tuberosum tissue by taking 50-100 mg of tissue and homogenising in 1% sodium metabisulphite solution. After filtering the extract through miracloth, the starch was collected by centrifugation, 1300 rpm for 5 minutes and then resuspended in 1 ml of water. Aliquots were taken and an equal amount of Lugol solution added to enhance the contrast of the starch granules. Suspensions were prepared for microscope imaging by placing onto a microscope slide. Representative micrographs were taken of the samples. The electronically captured images were then processed using suitable image analysis software, such as the package ‘Imagej’. This enabled a quantification of the size distributions of different starch samples to be made and compared.

Alternatively, samples of purified starch are either suspended in water and viewed with a light microscope or sputter-coated with gold and viewed with a scanning electron microscope such as a Phillips (Eindhoven, The Netherlands) XL30 Field Emission Gun scanning electron microscope at 3 kV.

Starch granules can be examined in tissues as well. For example, starch in tissues is stained using Lugol's solution (1% Lugol's solution, I-KI [1:2, v/v]; Merck). Starch can then be examined, for example, in longitudinal sections of tubers. Alternatively the starch can be further isolated subsequent to staining and suspended in water, and stained again with a few drops of Lungol's solution and examined microscopically.

The radii of the blue staining core of the starch granules and the total granule are measured microscopically using an ocular micrometer. If granules are ovoid in shape, both long radius and short radius measurements are taken. The radii of the blue-staining core and the total granule are determined by measuring individual, randomly chosen starch granules.

### Example 22

### Analysis of Starch Functionality

Preparation of Starch.

Starch was extracted from potato tubers by taking 0.5-1 kg of washed tuber tissue and homogenising using a juicerator chased with 200 ml of 1% Sodium bisulphite solution.

The starch was allowed to settle, the supernatant decanted off and the starch washed by resuspending in 200 ml of ice-cold water. The resulting starch pellet was left to air dry. Once dried the starch was stored at −20° C.

Alternatively, other methods can be utilized to isolate starch, for example, samples of tubers are first homogenized in extraction buffer (10 mM EDTA, 50 mM Tris, pH 7.5, 1 mM DTT, 0.1% Na2S2O5). The resulting fibrous substance is then washed several times with the extraction buffer and filtered. The filtrate is allowed to set at 4° C. and the supernatant is discarded after the starch granules have settled. Starch granules are then washed with extraction buffer, water, and acetone and dried at 4° C.

With maize and other cereal crops, seeds are soaked in 50 ml of a 20 mM sodium acetate, pH 6.5, 10 mM mercuric chloride solution. After 24 hr, the germ and pericarp are removed and 50 ml of fresh solution is added for an additional 24 hr. Endosperm is repeatedly homogenized for 1 minute intervals in a mortar and pestle, and freed starch granules are purified by multiple extractions with saline and toluene (Boyer et al., 1976, Cereal Chemistry 53: 327-337). Granular starch is washed three times with double distilled water, once with acetone, and dried at 40° C.

Viscometric Analysis of Starch.

Starch samples were analysed for functionality by testing Theological properties using viscometric analysis (rapid visco analyzer (RVA) or differential scanning calorimetry (DSC)). Viscosity of starches can also be measured by various other techniques. For example, a Rapid Visco Analyser Series 4 instrument (Newport Scientific, Sydney Australia) can be utilized with a 13 min profile where 2 g of starch are analyzed in water at a concentration of 7.4% (w/v) and the analysis used the stirring and heating protocol that suggested by Newport Scientific. For longer profiles, 2.5 g starch samples are used at a concentration of 10% (w/v). The sample is heated while stirring at 1.5° C. min⁻¹ from 50° C. to 95° C. for 15 min then cooled to 50° C. at 15 min⁻¹. Viscosity is measured in centipose (cP)

### Example 23

### Analysis of Fine Structure of Starch

Amylopectin Chain Length Distribution

One method for examining the fine structure of starch is ¹⁴C labeling of amylopectin chains to determine chain lengths. Extracted starch granules are suspended at 25 mg ml⁻ in medium comprising 100 mM Bicine (pH 8.50, 25 mM potassium acetate, 10 mM DTT, 5 mM EDTA, 1 mM ADP[U-¹⁴C] glucose at 18.5 GBq mol⁻¹ and 10 μl starch suspension in a total volume of 100 μl, for each sample. Samples are then incubated for 1 hour at 25° C. The incubation is terminated by addition of 3 ml 750 ml⁻¹ aqueous methanol containing 10 g 1-1 KCL (methanol/KCL). After incubation for at least 5 minutes at room temperature, starch is collected by centrifugation at 2000 g for 5 min. The supernatant is disgarded and the pellet is resuspended in 0.3 ml distilled water. The Methanol/KCL wash, centrifugation, and resuspension are repeated 2-4 times. The resulting pellets are dried at room temperature, dissolved with 50 μl 1M NaOH, and diluted with 50 μl distilled water. To determine the average length of amylopectin chains into which ¹⁴C was incorporated, products of incubation with ADP[U-¹⁴C] glucose are debranched with isoamylase and subjected to chromatography on a column of Sepharose CL-4B. The glucan eluding earlier from the column consists of longer chains than glucan eluding later from the column.

Another method for examining the fine structure of starch is chromatography without labeling. A 10 mg sample of isolated starch is dissolved in 100 ul 0.1 M NaOH for 1 hour at 95° C. The sample is diluted in 900 μL water, 150 μl 1 M soduim citrate (pH 5.0). The starch is then debranched by adding 300 units of isoamylase, or hydrolysed with 300 units of alpha-amylase, or beta-amylase for 24 hours at 37° C. A 100 ul aliquot sample of the hydrolysed samples is analyzed with chromatography. For example HPAE-PAD chromatography (Carbo PAC PA-100 column; Dionex, Idstein, Germany; flow 1 ml min⁻¹; buffer A: 150 mM NaOH; buffer B: 1 M sodium acetate in buffer A) with an applied gradient comprising 0-5 min 100% A; 5-20 min 85% A, 15% B, 20-35 min 70% A, 30% B (linear); 35-80 min 50% A, 50% B (convex).

Alternatively, HPLC chromatography is utilized, where partially hydrolyzed debranched starch samples in 0.01 N NaOH (5 mg/ml), and 2 ml are applied to a size exclusion column (Sephadex G-75, 1.5×100 cm). The mobile phase is 0.01 N NaOH and the flow rate is 0.6-0.9 ml/min. Samples are analyzed for total carbohydrate by the phenol-sulfuric acid test (Hodge and Hofreiter, 1962,Vol. 1, R. L. Whistler and M L Wolform (Eds.), Corporation. Version 7. Academic Press, New York, pp: 388-389) and the Park Johnson test for reduced ends (Porro et al., 1981, Anal Biochem. 118(2):301-6). Based on these to analyses the average chain length for each fraction is calculated.

Amylopectin is further characterized by measuring the low molecular weight to high molecular weight chain ratio (on a weight basis) according to the method of Hizukuri (Hizukuri, 1986, Carbohydrate Research, 147, 342-347).

An alternative method for analyzing amylopectin chains is gel electrophoresis. Starch samples are debranched with isoamylase, derivatised with fluorophore APTS, and subjected to gel electrophoresis in an Applied Biosystem DNA sequencer. Data are analized by Genescan software. The method allows for identification of authentic maltohexaose and maltoheptaose as well as a determination of percent molar differences and the degree of polymerization, distribution of chain lengths, between samples.

Amylose Content of Starch

Amylose percentages are determined by gel permeation chromatography according to Denyer et al. (Denyer et al., 1995, Plant Cell Environ 18:1019-1026) or by gel filtration analysis according to Boyer and Liu (Boyer and Liu, 1985, Starch Starke 37:73-79).

Alternatively, the amylose contents are determined spectrophotometrically in 1 to 2 mg isolated starch according to the iodometric method described by Hovenkamp-Hernelink et al. 1988. Amperometric titrations are performed according to Williams et al 1970 to determine the average amylose content per sample.

### Example 24

### cDNA Isolation from Barley

A database search using the Arabidopsis genes AT3g18660 and at1g77130, against an in-house database identified two barley sequences. The accessions corresponding to Genbank: BE438665 and Genbank: BE438754 showed significant similarity to the Arabidopsis PGSIP genes (9e-34). The sequences called Barley SEQ1 and Barley SEQ2 are shown in SEQ ID NO:s 16 and 18.

All publications, patents and patent applications mentioned in this specification are herein incorporated by reference into the specification to the same extent as if each individual publication, patent or patent application was specifically and individually indicated to be incorporated herein by reference.

Those skilled in the art will recognize, or through routine experimentation, will be able to ascertain many equivalents to the particular embodiments of the invention described herein. The claimed invention intends to encompass all such equivalents. Having herein above disclosed exemplary embodiments of the present invention, those skilled in the art will recognize that this disclosure is only exemplary such that various alternatives, adaptations, and modifications are within the scope of the invention, and are contemplated by the Applicants. Accordingly, the present invention is not limited to the specific embodiments as illustrated above, but is defined by the following claims.

What is claimed is:

1. An isolated nucleic acid molecule that:
(i) comprises a nucleotide sequence which encodes a polypeptide comprising the amino acid sequence of SEQ ID NO: 3, or a fragment thereof;
(ii) comprises a nucleotide sequence at least 40% identical to SEQ ID NOs: 1 or 2, or a complement thereof as determined using the BESTFIT or GAP programs with a gap weight of 50 and a length weight of 3; or
(iii) hybridizes to a nucleic acid molecule consisting of SEQ ID NOs: 1 or 2 under conditions of hybridization comprising washing at 60° C. twice for 15 minutes in 2×SSC, 0.5% SDS or a complement thereof.

2. The isolated nucleic acid molecule of claim 1, wherein the nucleic acid olecule comprises SEQ ID NOs: 1 or 2, or a complement thereof.

3. The isolated nucleic acid molecule of claim 1, comprising a nucleotide sequence selected from the group consisting of nucleotide residues 377 to 423, 516 to 592, 1039 to 1655, 1762 to 2536, and 2991 to 3264 of SEQ ID NO: 1.

4. An isolated nucleic acid molecule that:
(i) comprises a nucleotide sequence which encodes a polypeptide comprising the amino acid sequence of SEQ ID NO: 11, or a fragment thereof;
(ii) comprises a nucleotide sequence at least 70% identical to SEQ ID NO: 10, or a complement thereof as determined using the BESTFIT or GAP programs with a gap weight of 50 and a length weight of 3, wherein the nucleotide sequence does not encode the amino acid sequence set forth in SEQ ID NO: 35; or
(iii) hybridizes to a nucleic acid molecule consisting of SEQ ID NO: 10 under stringent conditions of hybridization, or a complement thereof, wherein the nucleotide sequence does not encode the amino acid sequence set forth in SEQ ID NO: 35.

5. The isolated nucleic acid molecule of claim 4, wherein the nucleic acid molecule comprises SEQ ID NO: 10, or a complement thereof.

6. An isolated nucleic acid molecule which encodes a polypeptide comprising the amino acid sequence that is at least 98% identical to SEQ ID NO: 9.

7. An isolated nucleic acid molecule thereof comprising the nucleotide sequence of SEQ ID NO: 8, or a complement thereof.

8. An isolated nucleic acid molecule that:
(i) comprises a nucleotide sequence which encodes a polypeptide comprising the amino acid sequence of SEQ ID NOs: 7, 13, 15, 17, 19, 21, 22, 24, 26, 28, 30, 32, 34, or a fragment thereof,
(ii) comprises a nucleotide sequence at least 70% identical to SEQ ID NOs: 4, 5, 6, 12, 14, 16, 18, 20, 23, 25, 27, 29, 31, 33, or a complement thereof as determined using the BESTFIT or GAP programs with a gap weight of 12 and a length weight of 4; or
(iii) hybridizes to a nucleic acid molecule consisting of SEQ ID NOs: 4, 5, 6, 12, 14, 16, 18, 20, 23, 25, 27, 29, 31, 33 under stringent conditions of hybridization, or a complement thereof.

9. The isolated nucleic acid molecule of claim 8, wherein the nucleic acid molecule comprises SEQ ID NOs: 4, 5, 6, 12, 14, 16, 18, 20, 23, 25, 27, 29, 31, 33, or a complement thereof.

10. A fragment of the isolated nucleic acid molecule of any one of claims 1-9, wherein the fragment comprises at least 40, 60, 80, 100 or 150 contiguous nucleotides of the nucleic acid molecule.

11. The isolated nucleic acid molecule of claim 1 comprising the nucleotide sequence of nucleotides 1-195 of SEQ ID NO: 2, or a complement thereof.

12. An isolated polypeptide comprising the amino acid sequence of amino acid residues 1-65 of SEQ ID NO: 3, or a fragment thereof.

13. An isolated polypeptide comprising:
(i) an amino acid sequence that is at least 70% identical to SEQ ID NO: 3 or a fragment thereof as determined using the BESTFIT or GAP programs with a gap weight of 12 and a length weight of 4;
(ii) an amino acid sequence encoded by the nucleic acid molecule of claim 1; or
(iii) an amino acid sequence of SEQ ID NO: 3.

14. An isolated polypeptide comprising:
(i) an amino acid sequence at least 70% identical to SEQ ID NO: 11, or a fragment thereof as determined using the BESTFIT or GAP programs with a gap weight of 12 and a length weight of 4;
(ii) an amino acid sequence encoded by the nucleic acid molecule of claim 4; or
(iii) an amino acid sequence of SEQ ID NO: 11.

15. An isolated polypeptide comprising:
(i) an amino acid sequence that is at least 98% identical to SEQ ID NO: 9 as determined using the BESTFIT or GAP programs with a gap weight of 12 and a length weight of 4;
(iii) an amino acid sequence encoded by the nucleic acid molecule of SEQ ID NO: 8, or a complement thereof; or (v) an amino acid sequence of SEQ ID NO: 9, or a fragment thereof.

16. An isolated polypeptide comprising:
(i) an amino acid sequence that is at least 70% identical to SEQ ID NOs: 7, 13, 15, 17, 19, 21, 22, 24, 26, 28, 30, 32, 34, or a fragment thereof as determined using the BESTFIT or GAP programs with a gap weight of 12 and a length weight of 4;
(ii) an amino acid sequence encoded by the nucleic acid molecule of claim 8;
(iii) an amino acid sequence of SEQ ID NOs: 7, 13, 15, 17, 19, 21, 22, 24, 26, 28, 30, 32, 34.

17. A fragment of a polypeptide comprising at least 8 amino acid residues, wherein said fragment is a portion of the polypeptide encoded by a nucleic acid molecule selected from the group consisting of exon I, exon II, exon III, exon IV and exon V of SEQ ID NO: 1.

18. A polypeptide comprising the amino acid sequence of SEQ ID: 3, 7, 9, 11, 13, 15, 17, 19, 21, 22, 24, 26, 28, 30, 32, 34 which further comprises one or more conservative amino acid substitution.

19. A fusion polypeptide comprising the amino acid sequence of any one of claims 12-18 and a heterologous polypeptide.

20. A fragment or immunogenic fragment of a polypeptide of any one of claims 12-18, wherein the fragment comprises at least 5, 8, 10, 15, 20, 25, 30 or 35 consecutive amino acids of the polypeptide.

21. An antibody that immunospecifically binds to a polypeptide of any one of the claims 12-18.

22. A method for making a polypeptide of any one of the claims 12-18, comprising the steps of:
(a) culturing a cell comprising a recombinant polynucleotide encoding the polypeptide of any one of claims 12-18 under conditions that allow said polypeptide to be expressed by said cell; and
(b) recovering the expressed polypeptide.

23. A complex comprising a polypeptide encoded by a nucleic acid molecule of any of claims 1-9 and a starch molecule.

24. The complex of claim 23, wherein the starch molecule comprises from 1 to 700 glucose units.

25. The complex of claim 23, wherein the starch molecule comprises branching chains of glucose polysaccharides.

26. A vector comprising a nucleic acid molecule of any one of claims 1-9.

27. An expression vector comprising a nucleic acid molecule of any one of claims 1-9 and at least one regulatory region operably linked to the nucleic acid molecule.

28. The expression vector of claim 27, wherein the regulatory region confers chemically-inducible, dark-inducible, developmentally regulated, developmental-stage specific, wound-induced, environmental factor-regulated, organ-specific, cell-specific, and/or tissue-specific expression of the nucleic acid molecule, or constitutive expression of the nucleic acid molecule.

29. The expression vector of claim 27, wherein the regulatory region is selected from the group consisting of a 35S CaMV promoter, a rice actin promoter, a patatin promoter, and a high molecular weight glutenin gene of wheat.

30. An expression vector comprising the antisense nucleotide sequence of a nucleic acid molecule of any one of claims 1-9, wherein the antisense sequence is operably linked to at least one regulatory region.

31. A genetically-engineered cell which comprises a nucleic acid molecule of any one of claims 1-9.

32. A cell comprising the expression vector of claim 27.

33. A cell comprising the expression vector of claim 30.

34. A genetically-engineered plant comprising the nucleic acid molecule of any of claims 1-9.

35. The genetically-engineered plant of claim 34 and progeny thereof, further comprising a transgene encoding an antisense nucleotide sequence.

36. The genetically-engineered plant of claim 31, further comprising a RNA interference construct.

37. A cell comprising a 35S CaMV constitutive promoter operably linked to a nucleic acid molecule of SEQ ID NO:2, or a rice actin promoter operably linked to a RNA interference construct comprising fragments of a nucleic acid molecule of SEQ ID NO:2, wherein said rice actin promoter confers expression of said fragments.

38. A method of altering starch synthesis in a plant comprising, introducing into a plant an expression vector of claim 27, such that starch synthesis is altered relative to a plant without the expression vector.

39. A method of altering starch synthesis in a plant comprising, introducing into a plant at least an expression vector of claim 30, such that starch synthesis is altered in comparison to a plant without the expression vector.

40. A method of altering starch granules in a plant comprising, introducing into a plant at least an expression vector of claim 27, such that the starch granules are altered in comparison to a plant without the expression vector.

41. A method of altering starch granules in a plant comprising, introducing into a plant at least an expression vector of claim 30, such that the starch granules are altered in comparison to a plant without the expression vector.

42. The method of claim 41, wherein starch granules are absent from leaves of the plant comprising at least an expression vector.

43. A plant part comprising a nucleic acid molecule of any of claims 1-9, wherein starch synthesis is altered.

44. The plant part of claim 43, wherein the part is a tuber, seed, or leaf.

45. The modified starch obtained from the plant part of claim 43, wherein the modification in the synthesized starch is selected from the group consisting of a ratio of amylose to amylopectin, amylose content, size of starch granules, quantity of size of starch granules, a ratio of small to large starch granules, and Theological properties of the starch as measured using viscometric analysis.
